(19) United States
(12) Patent Application Publication
(10) Pub. No.: US 2024/0415919 A1
(43) Pub. Date: Dec. 19, 2024

(54) COMPOSITIONS AND METHODS FOR TREATMENT OF AUTOIMMUNE DISORDERS AND CANCER

(71) Applicants: Dana-Farber Cancer Institute, Inc., Boston, MA (US); The Brigham and Women`s Hospital, Inc., Boston, MA (US)

(72) Inventors: Harvey CANTOR, Boston, MA (US); Hye-Jung KIM, Boston, MA (US); Hidetoshi NAKAGAWA, Boston, MA (US); John Yongjoon CHOI, Boston, MA (US); Jamil AZZI, Boston, MA (US)

(21) Appl. No.: 18/687,643

(22) PCT Filed: Aug. 30, 2022
(86) PCT No.: PCT/US2022/042020

§ 371 Date: Feb. 28, 2024

Related U.S. Application Data
(60) Provisional application No. 63/239,291, filed on Aug. 31, 2021.

(51) Int. Cl. A61K 38/08; A61K 39/00; A61P 35/00; A61P 37/06; C07K 7/06; C07K 16/28; G01N 33/564; G01N 33/68

(52) U.S. Cl. CPC A61K 38/08; A61K 39/001; A61P 35/00; A61P 37/06; C07K 7/06; C07K 16/2815; G01N 33/564; G01N 33/6854; A61K 2039/505; C07K 2317/31; G01N 2333/7051; G01N 2800/24; G01N 2800/52

(57) ABSTRACT

Aspects of the invention are drawn to compositions and methods for modulating CD8 Treg mobilization in the treatment of autoimmune disorders, rejection of transplanted organs and cancer.

### GOVERNMENT INTERESTS

This invention was made with government support under grant R01AI₀₃₇₅₆₂ awarded by the National Institutes of Health. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### CROSS-REFERENCE TO RELATED APPLICATIONS

The present application claims the benefit of priority to U.S. Provisional Application No. 63/239,291, filed Aug. 31, 2021, the contents of which are incorporated herein by reference.

### FIELD OF THE INVENTION

Aspects of the invention are drawn to compositions and methods for modulating CD8 Treg mobilization in the treatment of autoimmune disorders (e.g., CD8 Treg activation) and cancer (e.g., CD8 Treg depletion).

### SEQUENCE LISTING

The instant application contains a Sequence Listing which has been submitted electronically in ASCII format and is hereby incorporated by reference in its entirety. Said ASCII copy, created on [ ] is named [ ] and is [ ] bytes in size.

### BACKGROUND OF THE INVENTION

Although most CD8⁺ T-cells are equipped to kill cells infected by microbial invaders, a subset of these cells can regulate immune responses. Murine and human CD8 regulatory activity is invested in a small (<5% CD8 cells) subset that expresses a characteristic triad of surface receptors—CD44, CD122 and Ly49 (mouse)/KIR (human). These cells, herein termed CD8+ Treg or CD8 Treg cells, can eliminate activated CD4 T-cells through targeting of MHC class Ia or class Ib expressed by CD4+T-helper cells.

### SUMMARY OF THE INVENTION

Here we disclose methods for mobilizing (activating) or suppressing CD8 T regulatory cells (CD8 Treg), resulting in decreasing or increasing, respectively, CD4 T cell activity and immune responses in mammals. One type of CD8 Treg stimulator includes peptide superagonists for CD8 Treg and, when administered to or used to vaccinate mice, can reduce antibody mediated rejection (AMR) and allograft tissue damage. Peptide superagonists can also be used to treat autoimmune diseases. CD8 Treg can also be mobilized or depleted using certain antibodies. Antibodies that can deplete Treg cells can be used to treat cancer in mammals. The antibodies that bind to CD8 Treg can bind to unique molecules on CD8 Treg cells, including T cell receptors (TCRs).

Disclosed herein are methods for mobilizing a CD8 Treg cell in a mammal, comprising administering a CD8 Treg stimulator to the mammal. In some embodiments, the CD8 Treg cell stimulator can be a peptide/polypeptide agonist or superagonist of the CD8 T cell. In some embodiments, the peptide/polypeptide agonist or superagonist binds to a T cell receptor (TCR) on the CD8 Treg cell and to an MHC class Ib molecule on a CD4 T cell. In some embodiments, the CD8 Treg stimulator can be an antibody that binds to a CD8 Treg cell. In some embodiments, the antibody can bind to a TCR on the CD8 Treg cell. In some embodiments, the antibody can be a bispecific antibody. The methods can suppress CD4 cells. The methods can be used to treat autoimmune diseases and/or to reduce allograft rejection.

Disclosed herein are methods for depleting a CD8 Treg cell in a mammal, comprising administering a CD8 Treg cell depleter to the mammal. In some embodiments, the CD8 Treg cell deplete can be an antibody that binds to a CD8 Treg cell. In some embodiments, the antibody can bind to a TCR or other molecules on the CD8 Treg cell In some embodiments, the antibody can be a bispecific antibody. The methods can stimulate CD4 cells. The methods can be used to treat cancers.

Disclosed are CD8 Treg cell stimulator peptide/polypeptide agonists or superagonists. Disclosed are Treg cell stimulator antibodies. Disclosed are Treg cell depleter antibodies. Disclosed are pharmaceutical compositions of the peptide/polypeptide agonists or superagonists, the Treg cell stimulator antibodies and the Treg cell deplete antibodies. Disclosed are vaccine compostions of the peptide/polypeptide agonists or superagonists.

### BRIEF DESCRIPTION OF THE FIGURES

FIG. 1A-B. FIG. 1(A) provides a schematic depicting Ag (Qa-1-peptide)-specific recognition of target CD4 cells by CD8 Treg. Activation of alloreactive CD4 cells leads to upregulation of peptides presented on Qa-1. CD8 Treg express TCR that recognize Qa-1-peptide (pQa-1) complexes expressed by target CD4 cells, leading to suppression of alloreactive CD4 cells. FIG. 1(B) provides a schematic depicting the key molecular interactions between CD8 Treg and target CD4 cells. I) shows the TCR-pQa-1 interaction between CD8 Treg and target CD4 cells activates CD8 Treg. II) shows the engagement of pQa-1 by NKG2A receptor delivers inhibitory signal, resulting in diminished suppressive function. III) shows the stimulatory interaction between NKG2D and NKG2D-L to CD8 Treg suppressive function. IV) shows the blockade of inhibitory Ly49F signaling enhances CD8 Treg function.

FIG. 2A-C provides a representation of the identification of superagonists for FL9 T cells. This peptide library has mutations at each MHC anchoring position (p2,3,6,7 and 9) of the FL9 peptide. FL9 TCR⁺ hybridoma cells incubated with EL4 cells and each peptide variant and expression of CD69 was measured (panel A). Panel B shows the expression of CD69 (%), TCR downregulation and trogocytosis by FL9 TCR⁺ hybridoma cells plus each FL9 peptide variant. Panel C shows the expression of CD69 by FL9 TCR Tg T cells stimulated with selected FL9 peptide variants. Sequences of selected peptides are shown.

FIG. 3A-D provides a graphical representation depicting the FL9 Tg CD8 T cells suppression of activated CD4 T cells. In vitro: Con-A stimulated CD4 cells from WT B6, D227K KI and Qa-1 KO mice were co-cultured with 58C hybridoma expressing OT-I or FL9 TCR. After 3 days, the stimulation of OT-I TCR⁺ and FL9 TCR⁺ hybridoma by these activated CD4 cells was measured based on CD69 expression (panel A). OT-I or FL9 TCR+ hybridoma were co-cultured with Con-A stimulated CD4 cells from WT or ERAAP KO mice. Stimulation of OT-1 or FL9 TCR were measured based on CD69 expression by hybridoma cells (panel B). In vivo: WT or D227K mice were immunized with OT-II peptides in CFA. 7 days later, CD4 (CD4+CD25−) cells were isolated from immunized mice and transferred into WT B6 hosts with or without FL9 TCR Tg T cells followed by immunization with OTII/CFA (panels C,D). Detection of OT-II CD4 T cells in the spleen of hosts by OT-II tetramer (I-Ab/Ova) (panel C). Percentage and numbers of OT-II tetramer+CD4 cells recovered from adoptive hosts (panel D).

FIG. 4 provides a graphical representation showing a Qa-1-peptide yeast library, which identified peptides with superagonistic activity. FL9 T cells were stimulated with EL4 (Qa-1+) cells in the presence of surrogate peptides selected from peptide-Qa-1 yeast library for 3 days. Activation status of FL9 T cells was measured by CD69 expression. Surrogate peptides (#3, 4, 6,7,10,11) show increased stimulatory activity compared to FL9 variant FL9-68.

FIG. 5A-F provides graphical representations showing the characterization of Ly49F Knockout Models. Panel A provides a schematic showing Ly49F-KO have a disruption specifically in Ly49F, while Pan-KO mice have a disruption in all Ly49-related receptors. Both Ly49F and Pan-KO models lack Ly49F without a reduction in Helios expression (panel B), indicating that while Ly49F was deleted, these mice still possess a CD8 Treg compartment (panel C). PD-1 expression was reduced in both knockouts, indicating functional alterations within the CD8 Treg compartment (panel D). High-dimensional cytometry identified 20 distinct clusters of CD44-expressing CD8 T cells in the three genotypes tested (panels E,F).

FIG. 6 provides a schematic depicting CD8 Treg expression of inhibitory Ly49F receptor. Ly49F deficient CD8 Treg or blockade of inhibitory Ly49F signaling by anti-Ly49F Ab can enhance CD8 Treg function and expansion of self-reactive CD4 cells and generation of autoantibodies can be suppressed.

FIG. 7 provides a graphical representation showing in vivo Ly49 blockade may result in increased activation of CD8 Treg. WT B6 mice were injected with anti-Ly49F Ab (50 ug/mouse) or isotype control Abs at days 0, 2 and 4. Phenotype of CD8 Treg, identified by expression of Helios that is uniquely expressed by CD8 Treg, was analyzed at day 7. Representative data from two independent experiments is shown.

FIG. 8A-E provides a graphical representation of the generation of FL9 TCR Tg mice. Kb^{-/-} Db^{-/-} mice were immunized with FL9 peptide-loaded DC. FL9 specific CD8 T cells were sorted using PE and APC conjugated Qa-1/FL9 tetramers (panel A). TCR repertoire analysis showing predominance of Vα3.2 and Vβ5.1/5.2 among FL9-specific CD8 T cells (panel B). Comparison of FL9 tetramer binding with hybridoma expressing OT-1 (Vα2+VD5+) or FL9-specific TCR (Vα2+VD5+) (panel C). Confirmation of TCR specificity of FL9-Tg T cells (panel D). Expression of Ly49, NKG2A and NKG2D by FL9 TCR Tg T cells (panel E). Data are representative of two to three experiments.

FIG. 9A-B provides a graphical representation showing that the depletion of CD8 Treg enhances anti-tumor immune responses. WT B6 mice were inoculated with 2×10⁵ EL4 (Qa-1 WT) orEL4-Qa-1 KO cells. Mice received EL4 WT or EL4 Qa-1 KO cells were treated with four different conditions: 1) PBS/IFA+ isotype control Ab (mIgG1), 2) PBS/IFA+ anti-Ly49F Ab, 3) PBS/IFA+anti-Qa-1 Ab and 4) FL9.68/IFA+isotype Ab (mIgG1). 100 ug of FL9.68 peptide (day 0 and day 10), 50 ug of anti-Ly49F Ab and 100 ug of anti-Qa-1 Ab (day 0, 3 and 6) were injected respectively. A) Status of Ly49+CD8 cells in the blood at day 3 and 6 after tumor induction (panel A). Tumor growth in mice inoculated with EL4 WT (left) andEL4 Qa-1 KO (right) tumor cells until day 7. Expansion of CD8 Treg by FL9-68 SA vaccination leads to facilitated tumor growth, while depletion of CD8 Treg or blockade of Qa-1 interaction with its receptors result in slower tumor growth. This effect was not observed in mice inoculated with EL4-Qa-1KO tumor cells (panel B).

FIG. 10A-C provides a graphical representation showing FL9-68-dependent mobilization of CD8 Treg dampens autoantibody responses during MCMV infection. WT B6 mice were infected with MCMV (2×10⁵ pfu) and vaccinated with (100 ug/dose) FL9-68 in IFA or IFA alone at day 0, 8 and 12. Mice were bled at day 3, 10 and 18 and levels of anti-dsDNA Ab (panel A), viral titer (panel B) and frequency of activated CD8 Treg (panel C, Ly49+NKG2D+CD8) are shown.

FIG. 11A-D. FIG. 11(A) provides a graphical representation depicting the generation of FL9 superagonist. Hybridoma was engineered to express T cell receptor (TCR) restricted to FL9-Qa-1 while OT-1 TCR used as a control. FLP-TCR expressing hybridoma but not OT-1 TCR expressing hybridoma binds to FL9-Qa-1 tetramer (left panel). Library of modified FL9 peptides were generated and tested their capacity to activate (CD69) FL9-TCR hybridoma. FL9-68 was selected as a FL9-super-agnoist (FL9-SA) (right panel). FIG. 11(B) provides representative layouts of FL9-Qa-1 restricted CD8 Treg. Hosts immunized with FL9 show 8-fold expansion of FL9-Qa-1 tetramer binding CD8 Treg (CD44+CD122+Ly49+CD8+T). FIG. 11(C) provides a graphical representation depicting the immunophenotype of the spleen and draining lymph nodes. Tfh: follicular helper T cells (PD-1+CXCR5+CD4+T); GC B: Germinal Center B Cells (GL-7+FAS+B220+); PC: Plasma cells (CD138+B220); *: P<0.05; **: P<0.01; ***: P<0.001; n.s.: not significant. FIG. 11(D) provides a graphical representation showing the donor-specific antibody assay. X-axis indicates volume-to-volume ratio of recipient serum to donor splenocyte diluted in PBS at 10⁶ cells/mL.

FIG. 12 provides a graphical representation depicting the inhibition of GC response after heart graft transplantation by FL9-superagoinst peptide vaccine. B6 mice were vaccinated with FL9-68/IFA or IFA alone at day 0, 10, 13 and 16. At day 27, these B6 mice were transplanted with heart allograft from Balb/C mice along with CTLA-Ig injection. After one week, the frequency of TFH, GC B and plasma cells were analyzed in dLNs.

FIG. 13 provides a graphical representation showing that FL9 superagonist peptide vaccine inhibits AMR and allograft tissue damage. B6 mice were vaccinated with FL9-68/IFA or IFA alone at day 0, 10, 13 and 16. At day 27, these B6 mice were transplanted with heart allograft from Balb/C mice along with CTLA-Ig injection. After one week, levels of donor specific Abs were measured and C4d deposition in heart graft was assessed.

FIG. 14 shows molecules involved in the interaction of CD8+ T cells and CD4+ T cells, and also indicates approaches (I, II & III) for manipulating this interaction.

FIG. 15 provides a schematic depicting CD8 T cell maturation pathways, and a graphical representation depicting PD1 expression by FL9 thymocytes. Ly49 expression by developing thymocytes rescues deletion of PD1+FL9-TCR⁺ thymocytes.

FIG. 16 provides a graphical representation depicting LY49F-KO CD8 Treg memory pool replaced by CD8 effector T cells. CD8 T cell subsets are identified by high-dimensional cytometry.

FIG. 17 provides a schematic depicting the convergent evolution of LY49 and KIRs in humans and mice.

FIG. 18A-B provides a graphical representation depicting the KIR/Helios phenotype. FIG. 18(A) shows KIR⁺ subsets of CD8⁺ T cells (%). FIG. 18(B) shows Helios expression by KIR⁺ CD8⁺ T cells.

FIG. 19 provides a graphical representation showing the suppression of CD4+T_{FH} cell response by KIR⁺ CD8⁺ T cells.

FIG. 20 provides a schematic showing chronically-activated autoimmune CD4 T cells upregulate Qa-1/HLA-E+FL9 peptide.

FIG. 21 provides a representation depicting the docking of TCR onto MHC-peptide, and the division of labor by CDR1/CDR2/CDR3.

FIG. 22 provides a model for interaction of peptides with Qa-1 MHC (on CD4+ T cells) and TCR (on CD8+ Treg cells), and a strategy for screening for peptides having altered binding.

FIG. 23 provides a graphical representation showing a screen of peptides with mutations at MHC binding residues (FL9.8).

FIG. 24 provides a graphical representation showing a screen of peptides with mutations at TCR binding residues.

FIG. 25 provides a graphical representation showing a screen of peptides with mutations at TCR binding residues.

FIG. 26 provides a schematic depicting consensus motifs for FL9 superagonist synthetic peptides based on second generation screen of FL9 mutant libraries.

FIG. 27 provides a schematic showing FL9 TCRs are type II (conserved CDR1/CDR2 contact with Qa-1/HLA-E). 10/10 FL9 TCRs express TRAV 9N3. Cd8 Treg (FL9) TCRs all express common CDR1 and CDR2 sequences.

FIG. 28 provides a schematic showing FL9 TCRs are type II TCRs. 10/10 FL9 TCRs express TRBV 12-1/2.

FIG. 29 provides a schematic showing Hsp60-TCRs are type II TCRs (TCRα).

FIG. 30 provides a schematic showing Hsp60 TCRs are type II TCRs (TCRO).

FIG. 31 provides a schematic showing 6C5 TCR: Qa-1=proinsulin specific.

FIG. 32 provides a schematic showing TCRs: CMV induced/peptide specific (Type I).

FIG. 33 provides a schematic showing the conserved sequence of CDR1 and CDR2 are expressed by TCRs of CD8 Treg. Conserved CDR1 and CDR2 in TCR reactive to HLA-E-self-peptides. Non-canonical CDR1 and CDR2 react to HLA-E/foreign peptides. TCR8r targeted moiety includes toxins, CTL, and ADT-TCR⁸r.

FIG. 34 provides a schematic depicting the comparison of regulatory CD4 and CD8 T-cells.

FIG. 35 provides a schematic depicting the phylogenetic relationships of MHC class Ib molecules.

FIG. 36 provides a schematic showing the contribution of CD8 Treg to organ transplantation: use of Qa-1-DK mutant mice. Analysis of heart allograft transplantation in knockin mice containing a Qa-1 point mutation that disrupts Qa-1-peptide engagement of CD8 Treg is depicted.

FIG. 37 provides a schematic showing CD8 Treg-mediated immune suppression is important for long-term heart graft survival. Heart allograft rejection after immune suppression depends mainly on anti-graft allo or xeno antibodies.

FIG. 38 provides a representation showing increased activated CD4+T_{FH} and GC-B cells in B6.DK hosts after heart transplantation. Genetic disruption of pQa-1-TCR interaction enhances T_{FH} and B cell responses upon heart transplantation. (Left) Single-dose CTLA4-Ig heart transplant protocol using BALB/c heart allograft and C57BL/6 (WT) recipient. (Right) Comparison of activated CD4 (CD62L-CD44+CD4+), T_{FH} (PD-1+CXCR5+CD4+), Qa-1 MFI by total CD4 (blue) and T_{FH} (blue) and isotype-switched germinal center B cells (IgM-FAS+B220+) in recipient spleens (WT vs D227K mice) at day 28 post heart transplantation.

FIG. 39A-B provides graphical representations showing mutation of Qa-1 anchor residues identifies peptides for FL9 TCR⁺ CD8 Treg activation. Peptide-based mobilization of CD8 Treg to inhibit antibody-mediated heart rejection. (A) A library composed of 96 FL9 peptide variants (crude peptides) was generated by amino acid mutagenesis at the Qa-1 anchoring positions (p2, p3, p6, p⁷ and p9). FL9 TCR⁺ hybridomas were incubated with EL4 cells (Qa-1+) loaded with each FL9 peptide variant for 12 hrs before CD69 expression was measured as an indication of TCR stimulation (B). (C) Activation of FL9.2 T cells after stimulation with FL9 variants selected from library screen above. Dose-dependent activation of FL9 T cells was measured by culturing FL9.2 T cells with EL4 (Qa-1+) at various concentrations of indicated peptides (0, 1, 3 and 10 mg/ml).

FIG. 40 provides a graphical representation showing inhibition of GC response after heart graft transplantation by FL9-superagonist peptide vaccine.

FIG. 41 provides representation showing FL9s^{A} peptide vaccine inhibits production of donor specific Abs and graft tissue injury.

FIG. 42 provides a schematic showing the search for FL9 super-agonists. A Qa-1 yeast library was generated that expressed Qa-1 presenting 1×10⁸ peptides by random mutation of amino acids in each position, except p2 and p9 for 9mers and p2 and p10 for 10mers. The library was screened for binding to the FL9 TCR. Peptides bound to FL9 TCR were identified by sequencing of TCR-bound yeast clones.

FIG. 43 provides a graphical representation showing Qa-1/peptide yeast library identified peptides with superagonistic activity.

FIG. 44 provides a graphical representation showing amino acid motifs of peptides selected from library allows identification of endogenous self peptides that may activate FL9 T cells.

FIG. 45A-E and FIG. 45F-G. (A) provides representation showing the identification and isolation of Qa-1-FL9 specific T cells. WT B6 mice were immunized with Kb‘Db’ DC loaded with FL9 peptide at day 0, 8 and 15. At day 22, Qa-1-FL9-specific CD8 T cells were detected by tetramers (Qa-1-FL9-PE and Qa-1-FL9-APC) within CD44+CD122+Ly49+CD8 T cells. FIG. 45(B) provides representation showing TCR repertoire of Qa-1-FL9 Tet⁺ CD8 T cells. Single Qa-1-FL9-PE+Qa-1-FL9-APC+cells were sorted and subjected to sequencing for TCRα and TCRβ. 39 of TCRα and TCRβ pairs were analyzed based on their TCR V gene segments. Relative usage of TCRα and TCRβ V genes by these Tet⁺ single cells is depicted by donut chart. FIG. 45(C) provides representation showing TCR repertoire of Qa-1-Hsp60 Tet⁺ CD8 T cells. Single Qa-1-Hsp60-PE+Qa-1-Hsp60-APC+cells were sorted and subjected to sequencing for TCRα and TCRβ. Relative usage of TCRα and TCRβ V genes by these Tet⁺ single cells is depicted by donut chart. FIG. 45(D) provides a graphical representation showing Qa-1 dependent differentiation of FL9 T cells: tetramer mediated detection of TCR in 58C hybridoma transduced with FL9.2 and FL9.8 TCR (upper panel). Responsiveness of FL9.2 TCR and FL9.8 TCR expressing hybridoma upon stimulation with increasing dose of peptides measured by CD69 expression (lower panel). FIG. 45(E) provides a graphical representation showing measurement of Qa-1-FL9 binding affinity of FL9.2 and FL9.8 TCR. FL9.2 TCR⁺ and FL9.8 TCR⁺ hybridoma were labelled with Qa-1-FL9-PE tetramers and incubated in the presence of anti-Qa-1 Abs for the indicated time. Percentage of PE+cells were measured at different time points as a measurement of tetramer dissociation level. FIG. 45(F) provides a graphical representation showing Tg TCR⁺ cells in TCR⁺ thymocytes and percent of active Caspase-3+PD1+ cells in DP (CD4+CD8+) thymocytes in OT-I→WT B6, FL9.2 Tg 4 WT B6 BM chimera 8 wks after BM reconstitution. FIG. 45(G) provides a graphical representation showing Ki67 and CD44 expression by OT-I and FL9.2 TCR Tg CD8⁺ T cells was measured as an indication of Ag encounter in the spleen and liver of OT-I→WT B6 and FL9.2 Tg 4 WT B6 BM chimera 8 wks after BM reconstitution.

FIG. 46A-B provides a representation showing TCR repertoire of Qa-1/FL9 Tet+CD8 T cells. Single Qa-1-FL9-PE+Qa-1-FL9-APC+cells were sorted and subjected to sequencing for TCRα and TCRβ. 39 of TCRα and TCRβ pairs were analyzed based on their TCR V gene segments. Alignment of TCRα (A) and TCRβ (B) sequences obtained from Qa-1-FL9 Tet⁺ single cells.

FIG. 47A-B provides a representation showing TCR repertoire of Qa-1/FL9 Tet+CD8 T cells. Single Qa-1-FL9-PE+Qa-1-FL9-APC+cells were sorted and subjected to sequencing for TCRα and TCRβ. 39 of TCRα and TCRβ pairs were analyzed based on their TCR V gene segments. Alignment of TCRα (A) and TCRβ (B) sequences obtained from Qa-1-Hsp60 Tet⁺ single cells.

FIG. 48A-B and FIG. 48C-D provides a representation showing the generation of FL9 TCR⁺ hybridoma and analysis of their responsiveness to FL9 peptide. FIG. 48(A) provides a graphical representation showing the generation of Qa-1-FL9 specific hybridoma. 58Cα⁻β⁻ hybridoma were transduced with OT-I TCR or FL9 TCRs that were identified from the single cell TCR sequencing from Qa-1-FL9 tet+CD8 T cells. Correct folding and assembly of transduced TCRs and their specificity were tested by staining with anti-CD3, anti-TCR Vβ and Qa-1-Hsp60 or Qa-1-FL9 tetramers. FIG. 48(B) provides a graphical representation showing TCRs specific for Qa-1-FL9 may display distinct binding affinity. Qa-1-FL9 tetramer based dectetion of TCRs on 58C hybridomas that were transduced with individual TCR pairs isolated from Qa-1-FL9-specific single CD8 T cells. FIG. 48(C) provides a graphical representation showing differential responsiveness of Qa-1-FL9 specific TCRs to cognate peptide FL9. Responsiveness of FL9 TCR⁺ hybridoma to increasing concentrations of FL9 peptide depicted by surface CD69 expression. FIG. 48(D) provides a graphical representation showing the frequency of Vα3.2⁺ or Vβ5⁺ cells within Ly49+CD8 cells in spleen and LNs of WT B6, Qa-1.D227K KI and Qa-1 KO mice at 8 weeks of age (n=6/group).

FIG. 49A-B provides a graphical representation showing NKG2D expression by FL9 Tg T cells. Acquisition of NKG2D expression by FL9.2 (A) and FL9.8 (B) T cells with age. Percentage of NKG2D+cells within CD8αβ⁺ FL9 T cells from the spleen, LN and liver in FL9.8 TCR Tg mice at the age of 18 days, 9 wks and 4 mo (n=5/group).

FIG. 50A-C provides a graphical representation showing Qa-1-dependent differentiation of FL9 T cells. (A) Tg TCR⁺ cells (Vα3.2⁺ VD5+) in TCR⁺ thymocytes and percent of active Caspase 3⁺PD1⁺ cells in DP (CD4⁺CD8⁺) thymocytes in OT-I→WT B6, FL9.8 Tg 4 WT B6 BM chimera 8 wks after BM reconstitution. (B) Ki67 and CD44 expression by OT-I and FL9.8 TCR Tg CD8⁺ T cells was measured as an indication of Ag encounter in spleen and liver of OT-I→WT B6 and FL9.8 Tg 4 WT B6 BM chimeras 8 wks after BM reconstitution. C) TCR and CD8 expression on polyclonal non-Tg CD8 cells, OT-I and FL9.8 Tg T cells.

FIG. 51A-E and FIG. 51F provides representations showing Qa-1 dependent differentiation of FL9 T cells. FIG. 51(A) provides a graphical representation showing Qa-1 deficiency impairs development of self-reactive FL9 T cells. Frequency of Vα3.2⁺ Vβ5⁺ T cells in TCRb+cells from FL9.2 TCR Tg mice on Qa-1 WT and KO backgrounds (8 wks old). Representative FACS plots for the detection of Vα3.2⁺Vβ5⁺ cells in spleen are shown (left panel) (n=4/group). FIG. 51(B) provides a graphical representation showing Qa-1 deficiency and self-reactive markers. Expression of CD44 and NKG2D by FL9.2 T cells in spleen of WT.FL9.2 TCR Tg and Qa-1^{-/-}.FL9.2 TCR Tg mice. Representative FACS plots for NKG2D*CD44+ cells in the spleens of FL9.2 TCR Tg mice are shown on the left. FIG. 51(C) provides a graphical representation showing Qa-1 dependency for maintenance of FL9 T cells. CFSE-labelled FL9.2 T cells developed in Qa-1 WT or Qa-1 KO mice were transferred into irradiated (800 rads) Qa-1 WT, Qa-1 KO and D227K KI adoptive hosts. 7 days after transfer, Qa-1 WT or Qa-1 KO FL9.2 T cells were recovered from spleens of adoptive hosts. Numbers of FL9.2 T cells in the spleens of adoptive hosts are shown. FIG. 51(D) provides a graphical representation showing expression of Ki67 by FL9.2 T cells in the LNs of WT.FL9.2 TCR Tg and Qa-1^{-/-}.FL9.2 TCR Tg mice. FIG. 51(E) provides a graphical representation showing Qa-1 dependent activation and proliferation of FL9 T cells. Percentage of Qa-1 WT FL9.2 T cells that undergo >3 divisions in Qa-1 WT, Qa-1 KO and D227K KI hosts. FIG. 51(F) provides a graphical representation showing Qa-1 restricted CD8Treg: Vα3.2⁺V35+CD8 T cells. Frequency and phenotype of Vα3.2⁺Vβ5⁺ cells within Ly49⁺ CD8 cells in the spleens and LNs of WT B6, Qa-1.D227K KI and Qa-1 KO mice in 8 weeks age (n=6/group). Mean ±SEM is indicated. ****P<0.0001, ***P<0.001, **P<0.01, *P<0.05.

FIG. 52A-C provides a graphical representation showing Qa-1 dependent phenotype acquisition by FL9.8 Tg T cells. FIG. 52(A) provides a graphical representation showing reduced FL9 T cell development in the Qa-1 deficiency. Frequency of Vα3.2⁺Vβ5⁺ T cells in TCRO+cells from FL9.8 TCR Tg mice on Qa-1 WT and KO backgrounds. Representative FACS plots for detection of Vα3.2⁺VD5+ cells in spleen are shown on the left. FIG. 52(B) provides a graphical representation showing Qa-1 deficiency affects expression of markers for self-reactivity. Expression of CD44 and NKG2D by FL9.8 TCR Tg T cells in spleen and LNs of Qa-1 WT and Qa-1 KO mice. Representative FACS plots for NKG2D+CD44+ cells in spleens of FL9.8 TCR Tg mice are shown on the left. FIG. 52(C) provides a graphical representation showing expression of Ki67 by FL9.8 T cells in the LNs of WT.FL9.8 TCR Tg and Qa-1.FL9.8 TCR Tg mice at 8 wks of age.

FIG. 53 provides a graphical representation showing the detection of Vα3.2⁺Vb5⁺ CD8 cells in Ly49⁺ CD8 cells. The frequency of Vα3.2⁺Vβ5⁺ CD8 cells in the Ly49+ and Ly49⁻ CD8 cells.

FIG. 54A-B and FIG. 54C-D provides graphical representation showing FL9 Tg CD8 T cells recognize and suppress activated CD4 T cells. FIG. 54(A) provides a graphical representation showing in vitro: activated CD4 cells stimulate FL9 TCR in a Qa-1 dependent manner. In vitro: ConA stimulated CD4 cells from WT B6, Qa-1.D227K KI, KbDb KO and ERAAP KO mice were co-cultured with FL9.2 T cells isolated from FL9.2 TCR Tg mice. After 20 hrs, CD69 expression on FL9 Tg T cells were measured as a readout of TCR stimulation. FIG. 54(B) provides a graphical representation showing in vivo: FL9 Tg T cells suppress activated CD4 T cells (selectivity of the response). In vivo: WT or D227K mice were immunized with OT-II peptides in CFA. 7 days later, CD4 (CD4 CD25) cells were isolated from immunized mice and transferred into WT B6 hosts with or without FL9 TCR Tg T cells followed by immunization with OT-II/CFA. Detection of I-A^{b}/Ova₃₂₃-3₃₉ CD4 T cells in the spleen of hosts by I-A^{b}/Ova₃₂₃-3₃₉ tetramers (upper left). Percent and numbers of I-A^{b}/Ova₃₂₃-3₃₉ tetramer+(upper right) and I-A^{b}/Ova₃₂₃-3₃₉ tetramer activated (lower pannel) CD4 cells recovered from adoptive hosts (middle and right). FIG. 54(C) provides a graphical representation showing Vα3.2 T cell depletion and its impact on Ag specific CD4 cells. B6 WT and B6 D227K mice were immunized with Ova/CFA before injection with isotype or anti-Vα3.2 Ab on day 0, and boosted on day 8 with Ova/IFA along with Ab injection. Expression of Qa-1 on CD4 subsets and the frequency of I-Ab/Ova₃₂₃₋₃₃₉ tet+CD4 cells in the blood were assessed on day 15. FIG. 54(D) provides a graphical representation showing Qa-1 expression by I-A^{b}/Ova₃₂₃-3₃₉ tet+ and tet CD4 cells. Mean ±SEM is indicated. ****P<0.0001, ***P<0.001, **P<0.01, *P<0.05.

FIG. 55A-B provides graphical representation showing FL9 Tg CD8 T cells recognize activated CD4 T cells. (A) ConA-stimulated CD4 cells from WT B6, Qa-1.D227K KI, KbDb KO and ERAAP KO mice were co-cultured with FL9.8 T cells isolated from spleen and LNs of FL9.8 TCR Tg mice. After 20 hrs, CD69 expression on FL9 Tg T cells was measured as a readout of TCR stimulation. (B) ConA-stimulated CD4 cells from WT B6, D227K KI and Qa-1 KO mice were co-cultured with 58C hybridoma expressing OT-I or FL9.8 TCR. After 3 days, stimulation of OT-I TCR⁺ and FL9 TCR⁺ hybridoma by these activated CD4 cells was measured according to CD69 expression. (C) OT-I or FL9.8 TCR+hybridomas were co-cultured with Con-A-stimulated CD4 cells from WT or ERAAP KO mice. Stimulation of OT-1 or FL9 TCR was measured according to CD69 expression by hybridoma cells. Mean ±SEM is indicated. ****P<0.0001, ***P<0.001, **P<0.01, *P<0.05.

FIG. 56 provides graphical representation showing the depletion of Vα3.2⁺ T cells. WT B6 and D227K mice were immunized with Ova/CFA along with injection of rat IgG2b or anti-Vα3.2 Abs at day 0. These mice were boosted with Ova/IFA on day 8 and also Abs were injected. At day 15, the presence of Vα3.2⁺Vb5⁺ cells was assessed in total T, CD8 T and Ly49+CD8 T cells.

FIG. 57 provides graphical representation showing no in vivo stimulation of FL9.2 T cells by FL9 native peptides. CD45.1+B6 hosts were adoptively transferred with FL9.2 T cells and immunized i.p. with FL9 in CFA or no peptide (CFA alone) on day 0. Three days later, activation (CD69) proliferation of FL9.2 T cells (Ki67) was measured.

FIG. 58A-C and FIG. 58D-E provides representation showing identification of superagonists for FL9 T cells. FIG. 58(A) provides a schematic showing a library composed of 96 FL9 peptide variants (crude peptides) was generated by aa mutagenesis at the Qa-1 anchoring positions (p2, p3, p6, p⁷ and p9). FL9 TCR⁺ hybridomas were incubated with EL4 cells (Qa-1⁺) loaded with each FL9 peptide variant for 12 hrs and CD69 expression and TCR downregulation were measured as an indication of TCR stimulation. FIG. 58(B) provides a representation showing activation of FL9 TCR⁺ 58C hybridoma after stimulation with FL9 peptide variants. CD69 expression by FL9 TCR⁺ 58C hybridoma after stimulation with each FL9 peptide variant (left). Downregulation of TCR is shown as ATCR MFI based on calculation 100-(Testing TCR MFI/Control TCR MFI)×100 (%) (middle). Expression of Vα3.2 and Vβ5 on EL4 cells (trogocytosis) was measured (right). FIG. 58(C) provides a graphical representation showing activation of FL9.2 T cells after stimulation with FL9 variants selected from library screen above. Dose-dependent activation of FL9 T cells was measured by culturing FL9.2 T cells with EL4 (Qa-1⁺) at various concentrations of indicated peptides (0, 1, 3 and 10 μg/ml). FIG. 58(D) provides a graphical representation showing FL9-68 vaccination activates FL9 T cells in vivo. CD45.1⁺ B6 hosts were adoptively transferred with FL9.2 T cells and immunized i.p. with PBS, FL9, or FL9-68 in CFA on day 0. Six days later, proliferation of FL9.2 T cells (CD45.2⁺Vα3.2⁺VD5⁺) was measured by CFSE dilution (left). CD45.1⁺ B6 mice were immunized with Ova₃₂₃₋₃₃₉ peptide in CFA on day 6 and the frequency of I-A^{b}/Ova₃₂₃-3₃₉ Tet⁺ CD4 cells in activated (CD44⁺) CD4 cells was analyzed on day 14. Mean ±SEM is indicated. ****P<0.0001, ***P<0.001, **P<0.01, *P<0.05.

FIG. 58(E) CD45.1⁺ B6 mice that were vaccinated with FL9-68 in CFA or CFA alone on day 0 were immunized with Ova₃₂₃₋₃₃₉ peptide in CFA on day 6. The frequency of I-Ab/Ova₃₂₃₋₃₃₉ Tet⁺ CD4 cells in activated (CD44⁺) CD4 cells in the spleen and dLN was analyzed on day 14.

FIG. 59 provides a graphical representation showing the identification of superagonists for FL9 T cells. Activation of FL9.8 T cells after stimulation with FL9 variants selected from the library screen described in FIG. 58. Dose-dependent activation of FL9.8 T cells was measured by culturing FL9.8 T cells with EL4 (Qa-1*) at various concentrations of the indicated peptides (0, 1, 3 and 10 μg/ml).

FIG. 60A-F provides graphical representation showing superagonist peptide vaccination inhibits AMR in heart transplantation. FIG. 60(A) provides graphical representation showing expansion of FL9 specific CD8 Treg after FL9-SA vaccination. B6 mice were vaccinated with FL9-68 peptide in IFA or IFA alone on day 0 and sensitized with Balb/C skin on day 7. Mice were further vaccinated with FL9-68 in IFA or IFA alone on days 10, 13 and 16. Balb/C hearts were heterotopically transplanted to the abdominal cavity of B6 recipients. 250 ug of CTLA-4 Ig was administered i.v. post transplantation and recipients were analyzed on day 34. Frequency of Qa-1-FL9-Tet⁺ cells in spleens of IFA- or FL9-68/IFA-vaccinated B6 hosts is shown. FIG. 60(B) provides graphical representation showing inhibition of GC Ab response by FL9-68 immunization. Numbers of Tfh, GC B and plasma cells in dLNs of naïve mice or IFA- or FL9-68/IFA-vaccinated B6 recipients. FIG. 60(C) provides graphical representation showing Qa-1 expression by Tfh cells after heart transplantation. Qa-1 expression by total, naïve CD4 and Tfh cells in the spleen of B6 recipients of Balb/C heart graft. FIG. 60(D) provides graphical representation showing FL9-68 immunization inhibits production of DSA. Donor-specific Abs (IgG1) in naïve mice or IFA-, OT-I/IFA-, FL9/IFA- or FL9-68/IFA-vaccinated B6 recipients of heart grafts were measured in the serum collected on day 16. Balb/C donor splenocytes were incubated with serially diluted serum followed by detection with fluorescence-labelled anti-mouse IgG1 Abs. Statistical analysis was performed with Two Way-Anova involving mixed-effect analysis.

FIG. 60(E) provides representation showing FL9-68 vaccine inhibits graft tissue injury. C4d deposition in heart allografts. Tissue sections from heart grafts of B6 mice that were vaccinated with IFA alone or FL9-68/IFA were stained with anti-C4d Abs. FIG. 60(F) provides graphical representation showing FL9-68 vaccine promotes graft survival. Heart graft survival in mice vaccinated as indicated. Mean ±SEM is indicated. ****P<0.0001, ***P<0.001, **P<0.01, *P<0.05.

FIG. 61 provides a graphical representation showing responsiveness of recipient CD4 cells to allogeneic donor cells. CD4 cells were isolated from B6 recipients (at day X) that were transplanted with Balb/C heart and vaccinated with PBS or FL9-68 pepitdes. CFSE-labeled CD4 cells were co-incubated with irradiated donor splenocytes for 7 days and the level of proliferation was measured by CFSE dilution.

FIG. 62 illustrates primer sequences for amplication of TCRα and TCRβ sequences.

FIG. 63 illustrates human TCRa-TRAV8.3 as Vu gene homolog of mouse CD* Treg TCR (Vα3.2 [TRAV9]).

FIG. 64 illustrates anti-Ly49F optimized nucleotide sequences.

FIG. 65 illustrates anti-Ly49F variable region amino acid sequences.

FIG. 66 illustrates consensus motifs for FL9 superagonist synthetic peptides.

FIG. 67 illustrates KIR amino acid sequences.

FIG. 68 illustrates KIR and LY49 amino acid sequences.

FIG. 69 shows cancer and CD8 Treg depletion allowing for tumor vaccine therapy. Illustrated is tumor growth in mice that were inoculated with NT MC38 cells and treated with vaccine, anti-Ly49F Ab or vaccine+anti-Ly49F Ab. Irradiated C1.EZH2^{KO} MC38 cells were used as cancer cell vaccine (at day 10, 1×10⁶/mouse, s.c.). anti-Ly49F Abs (mIgG2a) was injected i.p. at day 10, 13 and 16. Anti-Ly49F Ab was engineered to express mIgG2a Fc region to allow for the deletion of Ly49F+cells. Tumor growth was monitored (left). The tumor volume is depicted for individual mouse in each treatment group at day 26 (right).

FIG. 70 shows cancer and CD8 Treg depletion. Tumor growth in B6 mice is shown that is inoculated with MC38 cells and treated with anti-Ly49 or anti-Vα3.2 Abs.

FIG. 71 depicts CD8 Treg depletion uncovering robust anti-tumor response. WT B6 mice were inoculated with MC38 tumor cells and vaccinated with CpG-ODN at day 3. Mice were treated with anti-Ly49F Ab at day 8, 11, 14, 17. Shown are tumor growth curves in the groups treated with isotype Ab (mIgG1), CpG, a-Ly49F Ab or CpG+a-Ly49F Ab.

FIG. 72 depicts cancer and CD8 Treg depletion allowing for tumor vaccine therapy.

FIG. 73 shows CD8 Treg depletion uncovering robust anti-tumor response.

FIG. 74A-B shows the CD8 T cell profile in tumorsd grown in mice that were treated with isotype or anti-Ly49F Abs. Percent of CD8 T cells among CD45+ cells and expression of GzmB in CD8 T cells (A) and percentage of CD8 Treg (CD44+CD122+Ly49+) in the CD45+ cells (B) are shown.

FIG. 75A-B shows NK and DC profile within the tumors grown in mice that were treated with isotype or anti-Ly49F Abs. (A) NK cell percentage among CD45⁺ cells and GzmB expression by NK cells within tumors. (B) Percentage of MDSC within CD11b⁺ cells and cDCs within CD11c⁺I-Ab⁺ cells.

FIG. 76A-B illustrates the tumor growth in B6 mice that were inoculated with B16 melanoma and treated with anti-Ly49F Ab (A). Number of cDC and MDSC within tumors that were treated with isotype or anti-Ly49F Abs (B).

FIG. 77 shows kidney transplantation, mobilization of CD8 Treg by synthetic peptide agonists prolongs kidney graft survival, and FL90SA (FL(-68) ameliorates AMR and prolongs graft survival in kidney transplantation. The left kidney of BALB/c mice (H-2^{d}) was recovered using a full-length ureter and transplanted into a B6 host (H-2^{b}). The ureter of the remaining native kidney was then ligated on post-operative day 2-4 to inhibit native kidney function. Surgical success was determined if mice survived seven days post-surgery (POD). Transplanted B6 hosts were treated intraperitoneally with FL9-SA (50 μg), or PBS emulsified in Adjuvant (Addavax™), once a week starting POD2. On day 20 following kidney transplantation (n=5-7/group), DSA levels in sera and capillary C4d deposition were measured. Survival of kidney allografts was measured by survival of recipients with absence of native kidney function. (Left) Donor-specific Abs (IgG1) in control or FL9-68/adj-vaccinated B6 recipients of kidney grafts. (Middle) Immunohistochemistry for C4d deposition. (Right) Survival of kidney allograft measured by survival of recipients with absence of native kidney function.

FIG. 78 is a schematic diagram illustrating development of CD8 Treg as compared to development of convention CD8 T cells (top) and illustrating CD8 Treg cells targeting CD4 T cells (bottom left), and superagonist peptide immunization to mobilize/activate CD8 Treg cells.

FIG. 79A-H illustrates example identification of Qa-1-FL9-specific TCR. (A) WT B6 mice were immunized with Kb^{-/-}Db^{-/-} DC loaded with FL9 peptide on days 0, 8 and 15. At day 22, Qa-1-FL9-specific CD8 T cells were detected by tetramers (Qa-1-FL9-PE and Qa-1-FL9-APC) within CD44⁺ CD122⁺ Ly49⁺ CD8 T cells. (B) TCR repertoire of Qa-1-FL9 Tet⁺ CD8 T cells. Single Qa-1-FL9-PE+Qa-1-FL9-APC+cells were sorted and subjected to sequencing for TCRa and TCRb. 39 of TCRa and TCRb pairs were analyzed based on their TCR V gene segments. Relative usage of TCRa and TCRb V genes by these Tet⁺ single cells is depicted by donut chart. (C) TCR repertoire of Qa-1-Hsp60 Tet⁺ CD8 T cells. Single Qa-1-Hsp60-PE⁺ Qa-1-Hsp60-APC⁺ cells were sorted and sequenced for TCRa and TCRb. Relative usage of TCRa and TCRb V genes by these Tet⁺ single cells is depicted by donut chart. (D) Frequency and phenotype of Vα3.2⁺Vb5⁺ cells within Ly49⁺ CD8 cells in the spleens and LNs of WT B6, Qa-1.D227K KI and Qa-1 KO mice in 8 weeks age (n=6/group). (E) Qa-1 dependent differentiation of FL9 T cells: tetramer-mediated detection of TCR in 58C hybridoma transduced with FL9.2 and FL9.8 TCR (upper panel). Responsiveness of FL9.2 TCR and FL9.8 TCR expressing hybridoma upon stimulation with increasing dose of peptides measured by CD69 expression (lower panel). (F) Measurement of Qa-1-FL9 binding affinity of FL9.2 and FL9.8 TCR. FL9.2 TCR⁺ and FL9.8 TCR⁺ hybridoma were labelled with Qa-1-FL9-PE tetramers and incubated in the presence of anti-Qa-1 Abs for the indicated time. Percentage of PE+cells were measured at different time points as a measurement of tetramer dissociation level. (G) Tg TCR⁺ cells in TCR⁺ thymocytes and percent of active-Caspase-3⁺ PD1⁺ cells in DP (CD4⁺ CD8*) thymocytes in OT-I→WT B6, FL9.2 Tg 4 WT B6 BM chimera 8 wks after BM reconstitution. (H) Ki67 and CD44 expression by OT-I and FL9.2 TCR Tg CD8⁺ T cells was measured as an indication of Ag encounter in the spleen and liver of OT-I→WT B6 and FL9.2 Tg 4 WT B6 BM chimera 8 wks after BM reconstitution.

FIG. 80A-H illustrates example Qa-1 dependent differentiation of FL9 T cells. (A) Frequency of Tg TCR⁺ cells in the TCR⁺ thymic cells in OT-I or FL9.2 TCR Tg mice (Vα2⁺Vb5⁺ for OT-I and Vα3.2⁺Vb5⁺ for FL9 T cells) and levels of Helios expression. (B) Frequency of Tg TCR⁺ cells in the TCR⁺ splenic cells in OT-I or FL9 TCR Tg mice and levels of Helios and Ly49 expression. (C) Frequency of FL9 TCR Tg T cells in the TCR+thymic cells in WT or Qa-1^{-/-} FL9 TCR Tg mice. Frequency of FL9 Tg T cells (Vα3.2⁺Vb5⁺) in the total thymocytes is shown in graph (right). (D) Frequency of Vα3.2⁺Vb5⁺ T cells in TCRb+spleen cells from FL9.2 TCR Tg mice on Qa-1 WT and KO backgrounds (8 wks old). Representative FACS plots for the detection of Vα3.2⁺Vb5⁺ cells in spleen are shown (left panel) (n=4/group). (E) Expression of CD44 and NKG2D by FL9.2 T cells in spleen of WT.FL9.2 TCR Tg and Qa-1^{-/-}.FL9.2 TCR Tg mice. Representative FACS plots for NKG2D+CD44⁺ cells in the spleens of FL9.2 TCR Tg mice are shown on the left. (F) Expression of Ki67 by FL9.2 T cells in the LNs of WT.FL9.2 TCR Tg and Qa-1^{-/-}.FL9.2 TCR Tg mice. (G) CFSE-labelled FL9.2 T cells developed in Qa-1 WT or Qa-1 KO mice were transferred into irradiated (800 rads) Qa-1 WT, Qa-1 KO and D227K KI adoptive hosts. 7 days after transfer, Qa-1 WT or Qa-1 KO FL9.2 T cells were recovered from spleens of adoptive hosts. Numbers of FL9.2 T cells in the spleens of adoptive hosts are shown. (H) Percentage of Qa-1 WT FL9.2 T cells that undergo >3 divisions in Qa-1 WT, Qa-1 KO and D227K KI hosts. Mean ±SEM is indicated. ****P<0.0001, ***P<0.001, **P<0.01, *P<0.05.

FIG. 81A-D illustrates an example of FL9 Tg CD8 T cell recognition and suppression of activated CD4 T cells. (A) in vitro: ConA stimulated CD4 cells from WT B6, Qa-1.D227K KI, KbDb KO and ERAAP KO mice were co-cultured with FL9.2 T cells isolated from FL9.2 TCR Tg mice. After 20 hrs, CD69 expression on FL9 Tg T cells were measured as a readout of TCR stimulation. (B) In vivo: WT or D227K mice were immunized with OT-II peptides in CFA. 7 days later, CD4 (CD4 CD25) cells were isolated from immunized mice and transferred into WT B6 hosts with or without FL9 TCR Tg T cells followed by immunization with OT-II/CFA. Detection of I-A^{b}/Ova₃₂₃-3₃₉ CD4 T cells in the spleen of hosts by I-Ab/Ova₃₂₃-3₃₉ tetramers (upper left). Percent and numbers of I-Ab/Ova₃₂₃-3₃₉ tetramer+(upper right) and I-A^{b}/Ova₃₂₃-3₃₉ tetramer activated (lower pannel) CD4 cells recovered from adoptive hosts (middle and right). (C) WT B6 and D227K mice were immunized with Ova/CFA and injected with isotype or anti-Vα3.2 Abs on day 0, before boosting on day 8 with Ova/IFA along with Ab injection. Expression of Qa-1 on CD4 subsets and frequency of I-Ab/Ova₃₂₃₋₃₃₉ tet+CD4 cells in blood were assessed on day 15. (D) Qa-1 expression by I-A^{b}/Ova₃₂₃-3₃₉ tet+ and tet−CD4 cells. Mean ±SEM is indicated. ****P<0.0001, ***P<0.001, **P<0.01, *P<0.05.

FIG. 82A-F illustrates an example of identification of superagonists for FL9 T cells. (A) A library composed of 96 FL9 peptide variants (crude peptides) was generated by aa mutagenesis at the Qa-1 anchoring positions (p2, p3, p6, p⁷ and p9). FL9 TCR⁺ hybridomas were incubated with EL4 cells (Qa-1⁺) loaded with each FL9 peptide variant for 12 hrs and CD69 expression and TCR downregulation were measured as an indication of TCR stimulation. (B) Activation of FL9 TCR⁺ 58C hybridoma after stimulation with FL9 peptide variants. CD69 expression by FL9 TCR⁺ 58C hybridoma after stimulation with each FL9 peptide variant (left). Downregulation of TCR is shown as ATCR MFI based on calculation 100-(Testing TCR MFI/Control TCR MFI)×100 (%) (middle). Expression of Vα3.2 and Vb5 on EL4 cells (trogocytosis) was measured (right). (C) Activation of FL9.2 T cells after stimulation with FL9 variants selected from library screen above. Dose-dependent activation of FL9 T cells was measured by culturing FL9.2 T cells with EL4 (Qa-1+) at various concentrations of indicated peptides (0, 1, 3 and 10 mg/ml). (D) CD45.1+B6 hosts were adoptively transferred with FL9.2 T cells and immunized i.p. with PBS, FL9, or FL9-68 in CFA on day 0. Six days later, proliferation of FL9.2 T cells (CD45.2⁺Vα3.2⁺Vb5⁺) was measured by CFSE dilution (left). (E) CD45.1+ B6 mice that were vaccinated with FL9-68 in CFA or CFA alone on day 0 were immunized with Ova₃₂₃₋₃₃₉ peptide in CFA on day 6. The frequency of I-Ab/Ova₃₂₃₋₃₃₉ Tet⁺ CD4 cells in activated (CD44⁺) CD4 cells was analyzed on day 14. (F) Comparison of high affinity Ab and auto-Ab responses in WT and Qa-1.D227K mice. WT B6 and Qa-1.D227K.KI mice were immunized with NP23-KLH/CFA and boosted with NP23-KLH/IFA on day 10. High affinity anti-NP responses were measured on day 15. Levels of anti-dsDNA Ab were measured on day 21. Mean ±SEM is indicated. ****P<0.0001, ***P<0.001, **P<0.01, *P<0.05.

FIG. 83A-F illustrates an example of superagonist peptide vaccination inhibiting AMR in heart transplantation. (A) B6 mice were vaccinated with FL9-68 peptide in IFA or IFA alone on day 0 and 7, followed by sensitization with Balb/C skin on day 10. Mice were further vaccinated with FL9-68 in IFA or IFA alone on days 10, 13 and 16. On day 27, Balb/C hearts were heterotopically transplanted to the abdominal cavity of B6 recipients. 250 ug of CTLA-4 Ig was administered i.v. post transplantation and recipients were analyzed on day 34. Frequency of Qa-1-FL9-Tet⁺ cells in spleens of IFA- or FL9-68/IFA-vaccinated B6 hosts is shown. (B) Numbers of Tfh, GC B and plasma cells in dLNs of naïve B6 mice or IFA- or FL9-68/IFA-vaccinated B6 recipients. (C) Qa-1 expression by total, naïve CD4 and Tfh cells in the spleen of B6 recipients of Balb/C heart graft. (D) Donor-specific Abs (IgG1) in naïve mice or IFA-, OT-I/IFA-, FL9/IFA- or FL9-68/IFA-vaccinated B6 recipients of skin grafts were measured in the serum collected on day 26, the day before heart transplantation. Balb/C donor splenocytes were incubated with serially diluted serum followed by detection with fluorescence-labelled anti-mouse IgG1 Abs. Statistical analysis was performed with Two Way-Anova involving mixed-effect analysis. (E) C4d deposition (blue) in heart allografts (upper panel). Tissue sections from heart grafts of B6 mice that were vaccinated with IFA alone or FL9-68/IFA were stained with anti-C4d Abs. H&E stain of heart allograft showing graft infiltrating lymphocytes (lower panel). (F) Heart graft survival in mice vaccinated with FL9-68 peptide as indicated. Mean ±SEM is indicated. ****P<0.0001, ***P<0.001, **P<0.01, *P<0.05.

FIG. 84A-H illustrates an example of FL9-SA (FL9-68) ameliorating AMR and prolonging graft survival in kidney transplantation. (A) Schematic of experimental design. (B) Frequency of FL9-Qa-1 specific CD8 (CD44⁺ CD122⁺ Ly49⁺) T cells in mice with or without FL9-68 immunization. (C) Frequency of Tfh, activated B cells and plasma cells in the graft recipients with or without FL9-68 immunization. (D) Donor-specific Abs (IgG1) in control or FL9-68/adj-vaccinated B6 recipients of kidney grafts. (E) Proliferation of activated CD4⁺ T cells from control or FL9-SA immunized recipients, when co-cultured with irradiated donor (BALB/c) splenocytes. (F) Gross anatomy of kidney allograft at day 20. (G) Immunohistochemistry for C4d deposition (blue). (H) Survival of kidney allograft measured by survival of recipients with absence of native kidney function. ***P<0.001, **P<0.01, *P<0.05.

FIG. 85A-B illustrates an example of the TCR repertoire of Qa-1-FL9 Tet⁺ CD8 T cells. Single Qa-1-FL9-PE+Qa-1-FL9-APC+cells were sorted and subjected to sequencing for TCRa and TCRb. TCRa and TCRb pairs were analyzed based on their TCR V gene segments. Alignment of TCRa (A) and TCRb (B) sequences obtained from Qa-1-FL9 Tet+single cells. TCR affinity to Qa-1-FL9 complex and TCR Va and VP gene usage by each Qa-1-FL9 Tet⁺ CD8 T cell are shown on the right. TRAV9N3 (Vα3.2) and/or TRBV12.1/2 (Vb5.1/2) expressing CD8 T cell clones are highlighted.

FIG. 86A-B illustrates an example of the TCR repertoire of Qa-1-Hsp60 Tet⁺ CD8 T cells. Single Qa-1-Hsp60-PE+Qa-1-Hsp60-APC+cells were sorted and subjected to sequencing for TCRa and TCR TCRa and TCRb pairs were analyzed based on their TCR V gene segments. Alignment of TCRa (A) and TCRb (B) sequences obtained from Qa-1-Hsp60 Tet⁺ single cells. TCR Va and VP gene usage by each Qa-1-Hsp60 Tet⁺ CD8 T cell are shown on the right. TRAV9N3 (Vα3.2) and/or TRBV12.1/2 (Vb5.1/2) expressing CD8 T cell clones are highlighted.

FIG. 87A-B illustrates an example of detection of Vα3.2⁺Vb5⁺ CD8 cells in Ly49+CD8 cells. (A) Gating strategy for Vα3.2⁺Vb5⁺ CD8 cell detection. Frequency ofVα3.2⁺Vb5+CD8 cells in the Ly49⁺ and Ly49⁻ CD8 cells. (B) Frequency of Vα3.2⁺ or Vb5⁺ cells within Ly49⁺ CD8 cells in spleen and LNs of WT B6, Qa-1.D227K KI and Qa-1 KO mice at 8 weeks of age (n=6/group).

FIG. 88A-C illustrates an example of Qa-1 dependent phenotype acquisition by FL9.8 Tg T cells. (A) Frequency of Vα3.2⁺Vb5⁺ T cells in TCRb+cells from FL9.8 TCR Tg mice on Qa-1 WT and KO backgrounds. Representative FACS plots for detection of Vα3.2⁺Vb5⁺ cells in spleen are shown on the left. (B) Expression of CD44 and NKG2D by FL9.8 TCR Tg T cells in spleen and LNs of Qa-1 WT and Qa-1 KO mice. Representative FACS plots for NKG2D+CD44⁺ cells in spleens of FL9.8 TCR Tg mice are shown on the left. (C) Expression of Ki67 by FL9.8 T cells in the LNs of WT.FL9.8 TCR Tg and Qa-1^{-/-}.FL9.8 TCR Tg mice at 8 wks of age.

FIG. 89A-D illustrates an example of depletion of CD8 Treg enhancing anti-tumor responses to MC38 carcinoma. (A) Growth curves of the MC38 tumor in groups of B6 mice treated with ether Ig isotype control or a-Ly49F monoclonal Ab (see Methods) alone or combined with vaccination with irradiated MC38 tumor cells prepared as described in Methods. B6 mice were inoculated subcutaneously with MC38 tumor cells (2×10⁵/mouse) on day 0 and treated with a-Ly49F or isotype control (30 mg/mouse) on days 8, 13 and 16 alone or combined with vaccination with irradidated MC38 cells on day 6. (B) Immune cell profiles in tumors from mice treated with isotype or a-Ly49F Abs on day 29 post tumor cell inoculation. The percentage of Ly49⁺ CD8 cells within CD122⁺ CD44⁺ CD8⁺ T cells, CD8 T cells within CD45⁺ cells, and the percent of GzmB+CD8 T and NK cells are shown as well as the percent of CD11′Gr1⁺ (MDSC) cells within CD45⁺ cells. (C) WT B6 mice were inoculated (s.c.) with MC38 cells (2×10⁵/mouse) and vaccinated with irradiated (2000 rads) MC38 cells on day 6 before treatment with isotype control or a-Vα3.2 Ab (80 mg/mouse) on days 10, 13 and 16. (D) MC38 growth in B6 mice after inoculation with MC38 cells (2×10⁵/mouse) on day 0, injected with CpG-ODN (50 mg; sub.cut.) on day 3 either alone or before treatment with a-Ly49F Ab (30 mg/mouse) on days 8, 11, 14 and 17. Each data point represents 5 mice/group with ***P<0.001, **P<0.01, *P<0.05. ****P<0.0001, ***P<0.001, **P<0.01, *P<0.05.

FIG. 90 illustrates an example of deletion of CD8 Treg by a-Ly49F antibody. B6 mice were injected i.p. with isotype (mIgG2a) or u-Ly49F Ab (clone HBF-719, 30 pg/mouse). 48 hrs after Ab treatment, the frequency of Ly49⁺ CD8 T cells in the blood, LNs, and spleen was measured by staining cells for TCR, CD8, CD44 and Ly49C/I/F/H (Ab clone: 14B11) expression. FACS plots are shown after gating on TCR⁺ CD8⁺ cells. Representative FACS plots and the summary of Ly49F+CD8 T cell frequency within CD8 T cells are shown.

FIG. 91A-C illustrates an example of anti-Ly49F Ab inhibiting growth of B16F10 melanoma cells and promotes early migration of cDC1 in the TME. B6 mice were inoculated subcutaneously with 2×105 B16F10 tumor cells on day 0 and injected with either u-Ly49F Ab or isotype control (100 μg/mouse) on days 6, 9 and 12. After monitoring tumor growth (A), mice were euthanized at day 17 and the proportions of cDC1 (CD11c+, XCR1+, CD103+) and the percent expressing KbDb+ in cDC (B) and MDSC (CD11b+, Gr1+) cells (C) were determined. 5 mice/group. *** P<0.001, * P<0.05.

### DETAILED DESCRIPTION OF THE INVENTION

Regulatory T (Treg) cells can function to regulate immune responses. One type of Treg cell, CD8+ Treg cells, or CD8 Treg cells (CD44+ CD122+ Ly49+ in mice; CD44+CD122+ KIR+ in humans), can suppress CD4+ T cells in a Qa-1- (mouse) or HLA-E-(human) restricted manner (MHC class Ib molecule). This CD4+ T cell suppression can be antigen specific due to CD8+ Treg cell recognition of CD4+ cells via T cell receptors (TCRs) in the context of Qa-1/HLA-E. Decreased CD8+ Treg activity can contribute to autoimmunity and inflammatory disease. Decreased CD8+ Treg activity can also contribute to antibody mediated rejection (AMR) of allografts. Antibody-mediated rejection (AMR) can be a barrier to successful solid organ transplantation. Increased CD8+ Treg activity can suppress these situations. Decreased CD8⁺ Treg activity can provide for increased tumor surveillance by the immune system.

Herein, approaches have been developed to regulate activity of CD8⁺ Treg cells. In some embodiments, CD8⁺ Treg activity can be increased. In some embodiments, superagonist peptides have been developed that can be used to mobilize/activate CD8⁺ Treg. In some embodiments, antibodies can also do this. In some embodiments, mobilization of CD8 Treg in this way can be used to suppress CD4⁺ T cells. In some embodiments, this can be used to suppress antibody-mediated rejection (AMR) of transplanted organs and other immune-mediated responses (e.g., autoimmunity).

Efficient targeting of Qa-1-FL9 (HLA-E-FL9) on CD4⁺ T cells by CD8 Treg after expansion of the Treg cells with peptide agonists is applicable to ameliorate multiple immune responses characterized by pathogenic antibodies in the context of autoimmune disease, organ transplantation and infection. Additionally, mobilization of CD8 Treg to regulate Ab-dependent immune response has an advantage over general immune suppression, which may leave the host immunologically compromised.

In some embodiments, CD8⁺ Treg activity can be decreased. In some embodiments, antibodies can be used to do this. In some embodiments, suppression/killing of CD8+ Treg using antibodies can increase activity of or relieve suppression of CD4+ T cells. In some embodiments, this can be used to increase immune responses, including tumor surveillance and anti-tumor activity in mammals.

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. For example, the term “about” is used herein to modify a numerical value above and below the stated value by a variance of 20 percent up or down (higher or lower).

### CD8 Treg Cells

CD8 Treg regulate immune responses against pathogens and self-antigens by eliminating chronically activated CD4 cells that upregulate Qa-1/HLA-E on their surface. Recognition of Qa-1-self-peptide on target cells by CD8 Treg can suppress pathogenic CD4 cells, but CD8 Treg expansion and mobilization are constrained by molecular mechanisms that constrain excessive or inappropriate CD8 Treg activation. Herein are disclosed new strategies that allow both antigen-specific and antigen-nonspecific therapeutic mobilization of CD8 Treg in the context of transplant rejection, autoimmune disease and cancer.

Murine and human CD8 regulatory activity make up a small (<5%) subset of total CD8 T cells that express a characteristic triad of surface receptors: CD44, CD122 and Ly49 (mouse)/KIR (human). Analysis of autoimmune disorders has revealed that these CD8 T regulatory cells (CD8 Treg) inhibit disease through targeting of MHC class Ia or class Ib expressed by CD4⁺ T-helper cells.

Generally, CD8 Treg can express CD8, Ly49F, CD44 and CD122 (i.e., in mice) or CD8, iKIR, CD44 and CD122 (i.e., in humans). Ly49F is a subtype of the Ly49 receptor family. Ly49 receptors are type II C-type lectin-like membrane glycoproteins. KIR receptors are expressed by human cells and the functional homolog of Ly49 receptors in mice.

Although recognition of MHC-E (human HLA-E or murine Qa-1)-peptide complexes expressed by target CD4 cells is required for regulatory activity, the identity of TCRs that recognize class Ib (Qa-1) target ligands and associated self-peptides is not known. Herein, we disclose such TCRs.

Analysis of a panel of more than 30 independent TCRs expressed by Qa-1-restricted CD8 T cells specific for two structurally-distinct self-peptides (FL9: FYAEATPML, Hsp60p216: GMKFDRGYI) revealed predominant usage of TRAV9N3 and TRBV12-1/2 genes encoding TCR Vα3.2/Vb5.1. Development and function of Ly49F* Vα3.2/Vb5.1⁺ was almost completely abrogated in Qa-1-deficient mice, indicating that the Qa-1-restricted subset of CD8 Treg is confined to CD8 cells expressing the Vα3.2/Vb5.1 TCR.

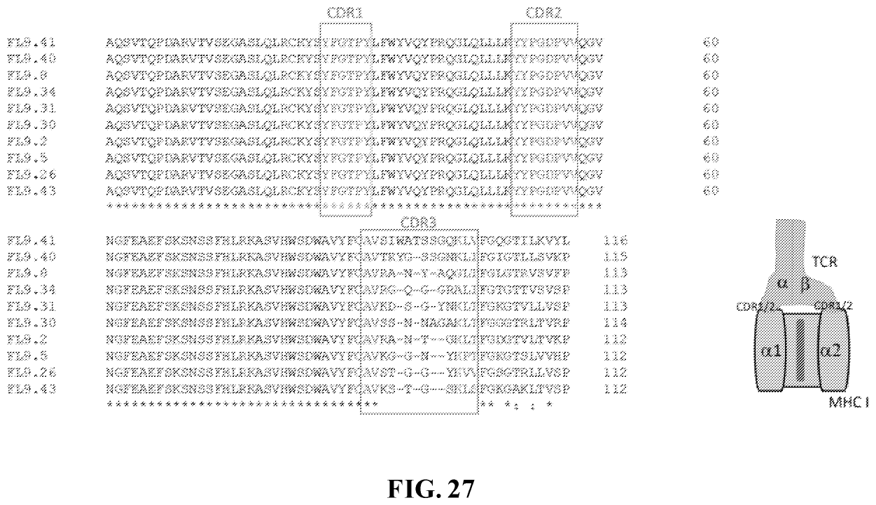

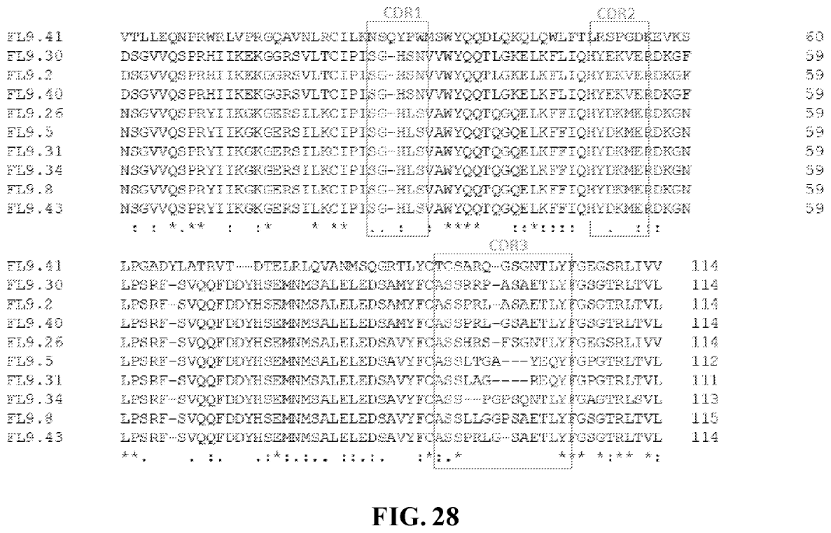

In some embodiments, the TCRs are shown in FIGS. 27-28 (e.g., specific for the FL9 peptide) and 29-30 (e.g., specific for the Hsp60 peptide) herein. In some embodiments, a CD8 Treg TCRα CDR1 sequence can be at least 90, 91, 92, 93, 94, 95, 96, 97, 98 or 99% identical to YFGTPYY; a CD8 Treg TCRα CDR2 sequence can be at least 90, 91, 92, 93, 94, 95, 96, 97, 98 or 99% identical to YYPGDPVV; a CD8 Treg TCRα CDR3 sequence can be at least 90, 91, 92, 93, 94, 95, 96, 97, 98 or 99% identical to AVSIWATSSGQKLV; AVTRYGSSGNKLI; AVRANYAQGLT; AVRGQGRALI; AVKDSGYNKLT; AVSSNNAGAKLT; AVRANTGKLT; AVKGGNYKPT; or AVKSTGSKLS; a CD8 Treg TCRP CDR1 sequence can be at least 90, 91, 92, 93, 94, 95, 96, 97, 98 or 99% identical to NSQYPW; SGHSN; or SGHLS; a CD8 Treg TCRP CDR2 sequence at least 90% identical to LRSPGDK; HYEKVER; or HYDKMER; or a CD8 Treg TCRP CDR3 sequence can be at least 90, 91, 92, 93, 94, 95, 96, 97, 98 or 99% identical to TCSARQGSGNTLY; ASSRRPASAETLY; ASSPRLGSAETLY; ASSHRSFSGNTLY; ASSLTGAYEQY; ASSLAGREQY; ASSPGPSQNTLY; ASSLLGGPSAETLY; or ASSPRLGSAETLY.

In some embodiments, a CD8 Treg TCRα CDR1 sequence can be at least 90, 91, 92, 93, 94, 95, 96, 97, 98 or 99% identical to ATSIAYPN, or YFGTPL; a CD8 Treg TCRαCDR2 sequence can be at least 90, 91, 92, 93, 94, 95, 96, 97, 98 or 99% identical to KVITAGQ; or KYYPGDPV; a CD8 Treg TCRα CDR3 sequence can be least 90, 91, 92, 93, 94, 95, 96, 97, 98 or 99% identical to ALGEASSGSWQL; AVSSNYNVL; AVSRANTGKL; AVSKDSGYNKL; or AVSKSTGSKL; a CD8 Treg TCRP CDR1 sequence can be least 90, 91, 92, 93, 94, 95, 96, 97, 98 or 99% identical to TNNHN; ISGHL; or LSGHS; a CD8 Treg TCRP CDR2 sequence can be at least 90, 91, 92, 93, 94, 95, 96, 97, 98 or 99% identical to; SYGAGS; HYDKME; or HYEKVE; or a CD8 Treg TCRP CDR3 sequence can be at least 90, 91, 92, 93, 94, 95, 96, 97, 98 or 99% identical to CASGTGDERL; CASSLVSGSAEQ; CASSLAGREQ; CASSLGQGNYAEQ; or CASSRANYEQ.

The above listing of TCRs is not meant to be limiting. In embodiments, the CD8 Treg TCRs can bind self-peptides. The TCRs can bind the self-peptides within the context of an MHC Ib molecule. The MHC Ib molecule can be Qa-1 or HLA-E. The MHC Ib molecule can be expressed on CD4 T cells. Generally, the MHC Ib molecule is present on CD4 T cells.

In some embodiments, the TCRs or the CDRs therefrom, as described above, can be engineered to be expressed on or in various cells, including cell lines or primary cells. In some embodiments, the TCRs/CDRs can be expressed on hybridoma cells or on chimeric antigen receptor T cells (CAR-T cells). In some embodiments, the TCRs/CDRs can be expressed in various transgenic animals. In some embodiments, the TCRs/CDRs can be expressed in transgenic mice. The cells and transgenic animals are part of the disclosed invention.

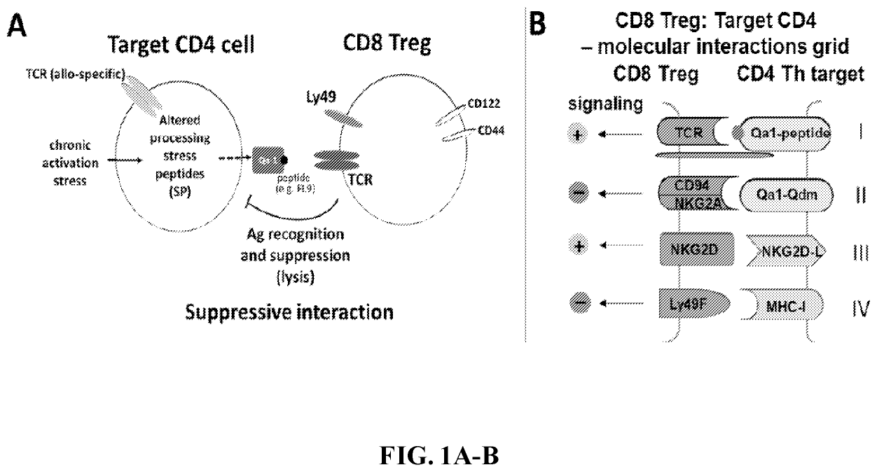

Other properties of the CD8 Treg cells disclosed herein can be seen, for example, in FIGS. 1A-B, 6, 14, 34, 36 and 78.

In some embodiments, the TCRs disclosed herein can be made to be expressed on various cells or transgenic animals. In some embodiments, a hybridoma can be engineered to express a TCR. In some embodiments, a transgenic animal (e.g., mouse) can be engineered to express a TCR.

### CD8 Treg Agonists

Herein, agonists of CD8 Treg cells or CD8 Treg stimulators can mobilize or activate these cells. In some embodiments, the Treg stimulators can be peptides or polypeptides. In some embodiments, the Treg stimulators can be antibodies.

Peptide agonists can be of a variety of types and have a variety of amino acid sequences. In some embodiments, the disclosed peptide agonists are or are derived from self-peptides. The self-peptides generally can bind to molecules expressed on CD8 Treg cells.

In some embodiments, FL9 and Hsp60 peptides have been identified that stimulate the CD8 Treg cells. In some embodiments, superagonist (SA) variants of the these self-peptides have been engineered to express potent CD8⁺ Treg cell stimulatory activity in association with Qa-1b (or HLA-E). Vaccination with the superagonist peptides can lead to efficient mobilization of CD8 Treg and inhibition of antibody-mediated allograft rejection, autoimmune diseases, and the like.

Disclosed herein is an approach based on the application of superagonist self-peptides that can efficiently expand CD8 Treg, reduce germinal center (GC) responses and suppress antibody responses. This approach results in mobilization of CD8 Treg and reduces Ab-mediated injury to allogeneic organ transplants.

Based on previous mass-spectrometry studies, two SPs (superagonist peptides) were selected—FL9 and Hsp60p216—that associate with Qa-1 under immunologic stress conditions. We then sorted FL9-tetramer binding CD8 Tregs, sequenced their TCR, and expressed on hybridoma. This is our hybridoma system. We also generated a library of modified FL9 sequences and compared their antigenicity using the TCR engineered hybridoma. After selecting FL9-SA (FL9-superagonist peptides), we performed BALB/c to B6 skin transplantation with or without Hsp60p216 and FL9-SA, followed by heart transplantation.

We successfully generated FL9-SA using our TCR engineered hybridoma system.

Immunization with SPs (superagonist peptides) significantly expanded SP-Qa-1 tetramer binding CD8 Treg. Compared to the control group, hosts treated with SPs during sensitization showed a significant reduction in Tfh (T follicular helper cells) and mature B cells including plasma cells. FL9-SA was more efficacious than Hsp60p2l6. Also, donor-specific antibody (DSA) was significantly decreased in SP-treated groups, resulting in protection of heart allografts.

Eliciting CD8 Treg response with Qa-1-associating SPs subdued germinal center reaction and DSA formation. Especially, the super-agonist that we generated showed good biological efficacy in mobilizing CD8 Treg. Exploiting the mechanism of CD8 Treg through the study of Qa-1-associating peptides is a new strategy to suppress AMR, which lacks effective therapeutic options.

In some embodiments, the agonist/superagonist peptides/polypeptides can bind to TCRs on CD8 Treg cells. Generally, the peptides/polypeptides can bind to the TCRs within the context of an MHC Ib molecule, like Qa-1 and/or HLA-E. The MHC Ib molecule can be on a cell, like a CD4 T cell, for example. Generally, the peptides are from proteins that are “self” proteins (e.g., from mice, from human). Generally, interactions of the CD8 Treg cells with CD4 T cells involves multiple molecular reactions, some of which are illustrated in FIGS. 1A-B, 6, 14, 36 and 78, for example.

In some embodiments, the agonist/superagonist peptides/polypeptides can include amino acid sequences FSNEATLML; WYADVTPAL; or an amino acid sequence 88%, 89%, 90%, 91%, 92%, 93%, 94%, 95%, 96%, 97%, 98% or 99% identical thereto.

In some embodiments, the agonist/superagonist peptides/polypeptides can include an amino acid sequence: FYAEATLML (FL9-68); or an amino acid sequence 88%, 89%, 90%, 91%, 92%, 93%, 94%, 95%, 96%, 97%, 98% or 99% identical thereto.

In some embodiments, the agonist/superagonist peptides/polypeptides can include an amino acid sequence: IMLDTEIRL (BO-1); FMNDALLFL (BO-2); FMEEYMPFL (BO-
3); FMEDAGPRL (BO-5); WMSEDHTLL (BO-6); VMQDEKSRL (BO-9); ISSEDGVPL (BO-10); or an amino acid sequence 88%, 89%, 90%, 91%, 92%, 93%, 94%, 95%, 96%, 97%, 98% or 99% identical thereto.

In some embodiments, the agonist/superagonist peptides/polypeptides can include an amino acid sequence: FISDSFFFL (Endo 9); FYAEGTTML (MTb); or an amino acid sequence 88%, 89%, 90%, 91%, 92%, 93%, 94%, 95%, 96%, 97%, 98% or 99% identical thereto.

In some embodiments, the agonist/superagonist peptides/polypeptides can include an amino acid sequence: FYAEATPML (FL9) or an amino acid sequence 88%, 89%, 90%, 91%, 92%, 93%, 94%, 95%, 96%, 97%, 98% or 99% identical thereto.

Generally, these amino acid sequences can stimulate CD8 Treg and/or suppress CD4 T cells in a mammal.

In some embodiments, the peptides/polypeptides can be attached to/conjugated to a lipophilic albumin binding tail conjugate, for example.

In some embodiments, the Treg stimulators can be antibodies. These and other antibodies are described in the following section.

### Antibodies to CD8 Treg

Disclosed herein are antibodies specific for and that bind to CD8 Treg cells and molecules expressed by CD8 Treg cells.

Herein, “antibody” can refer to a molecule or molecules that binds an antigen. Herein, “antibody” can refer to all types of antibodies, fragments and/or derivatives. Antibodies include polyclonal and monoclonal antibodies of any suitable isotype or isotype subclass. Herein, antibody can refer to, but not be limited to Fab, F(ab′)2, Fab′ single chain antibody, Fv, single chain, mono-specific antibody, bi-specific antibody, tri-specific antibody, multi-valent antibody, chimeric antibody, canine-human chimeric antibody, chimeric antibody, humanized antibody, human antibody, CDR-grafted antibody, shark antibody, nanobody (e.g., antibody consisting of a single monomeric variable domain), camelid antibody (e.g., from the Camelidae family) microbody, intrabody (e.g., intracellular antibody), and/or de-fucosylated antibody and/or derivative thereof. Mimetics of antibodies are also provided. In embodiments, the antibody can have a heavy chain constant region, a light chain constant region, an Fc region/portion, or a combination thereof. In embodiments, the antibody can be fully human, humanized, or a chimera. The antibody or fragment can be monoclonal. In some embodiments, antibody can be used in a CAR-T construct.

In some embodiments, the antibody can have a therapeutic moiety (e.g., a toxin), an imaging moiety (e.g., a fluorophore, chromophore, or a combination thereof), a capture moiety (e.g., GST tag, His-Tage, or a combination thereof) or a combination thereof).

The antibodies can or cannot have an Fc portion that can bind to an Fc receptor (FcR). The FcR can be present on effector cells, including natural killer (NK) cells or macrophages. In some embodiments, the Fc portion of the antibody can bind to FcRs including an Fc-gamma receptor (FcγR), an Fc-alpha receptor (FcuR), or an Fc-epsilon receptor (FcFR). The FcγR can can include at least FcγRI, FcγRII, or FcγRIII. In some embodiments, the Fc portion of the antibody can be modified to better bind to an FcR as compared to an Fc portion that has not been modified.

Herein, the disclosed antibodies generally can have the effect of stimulating or mobilizing CD8 Treg (e.g., perhaps in a way similar to agonist/superagonist peptides described earlier). Other antibodies, disclosed herein, can have the effect of repressing or depleting CD8 Treg cells. In some embodiments, the antibodies that repress CD8 Treg cells kill or mediate killing of the cells. In some embodiments, antibodies that repress/mediate killing of CD8 Treg cells may bind effector cells (e.g., NK cells, macrophages) such that the effector cells mediate the repression/killing of the cells. In some embodiments, antibodies that bind to a molecule (e.g., Ly49, iKIR, TCRs on CD8 Treg) can be screened for a functional effect of the binding, like mobilizing CD8 Treg cells or depleting CD8 Tregs cells, for example.

In some embodiments, the antibodies are specific for binding to molecules expressed by CD8 Treg cells that identify CD8 Treg cells. In some embodiments, the antibodies can be specific for Ly49 (mouse) and/or iKIR (human). In some embodiments, the antibodies can be specific for TCRs expressed on specific CD8 Treg cells. In some embodiments, the antibodies can identify a combination of molecules expressed by a CD8 Treg cell (e.g., two or all of LY49/iKIR, CD8, TCR). In some embodiments, these antibodies may be multispecific antibodies, like bispecific or trispecific antibodies and the like. In some embodiments, bispecific antibodies can bind to iKIR (and/or Ly49) and CD8; iKIR (and/or Ly49) and a CD8 Treg cell TCR; CD8 and a CD8 Treg cell TCR; or to iKIR (and/or Ly49), CD8 and a CD8 Treg cell TCR.

Generally, the TCRs on CD8 Treg cells to which the disclosed antibodies can bind are TCRs that can bind self-peptides. Generally, the peptides are bound by the TCRs in the context of MHC molecules that can bind self-peptides. In some embodiments, these MHC molecules can be MHC Ib molecules, like Qa-1 or HLA-E. Generally, the TCRs can bind to any self-peptides. Some examples of self-peptides can include FL9, amino acid sequence-modified FL9, Hsp60p2l6, amino acid sequence-modified Hsp60p2l6, and the like (discussed in section on CD8 Treg Agonists). In various embodiments, the TCRs can bind to any of the peptides described in the previous section, titled “CD8 Treg Agonists.”

In embodiments, the antibodies that bind to the TCRs can bind to the α or β chain of the TCRs. In embodiments, the antibodies can bind to CDRs of the TCRs. In embodiments, the antibodies can bind to CDR1, CDR2, or CDR3 of the α or β chain of the TCRs. In embodiments, the CDRs can be any of the CDRs illustrated in FIGS. 27, 28, 29, or 30. In embodiments, the CDRs can be any of the CDRs described in the section of this application titled “CD8 Treg Cells.”

Regarding example properties of antibodies disclosed herein, “recombinant” as it pertains to polypeptides (such as antibodies) or polynucleotides refers to a form of the polypeptide or polynucleotide that does not exist naturally, a non-limiting example of which can be created by combining polynucleotides or polypeptides that would not normally occur together. “Polypeptide” as used herein can encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids, can refer to “polypeptide” herein, and the term “polypeptide” can be used instead of, or interchangeably with any of these terms. “Polypeptide” can also refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology but is not necessarily translated from a designated nucleic acid sequence. It may be generated in any manner, including by chemical synthesis. As to amino acid sequences, one of skill in the art will readily recognize that individual substitutions, deletions or additions to a nucleic acid, peptide, polypeptide, or protein sequence which alters, adds, deletes, or substitutes a single amino acid or a small percentage of amino acids in the encoded sequence is collectively referred to herein as a “conservatively modified variant”. In some embodiments the alteration results in the substitution of an amino acid with a chemically similar amino acid. Conservative substitution tables providing functionally similar amino acids are well known in the art. Such conservatively modified variants of antibodies disclosed herein can exhibit increased cross-reactivity in comparison to an unmodified antibody.

For example, a “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is replaced with another amino acid residue from the same side chain family. In another embodiment, a string of amino acids can be replaced with a structurally similar string that differs in order and/or composition of side chain family members.

Some embodiments also feature antibodies that have a specified percentage identity or similarity to the amino acid or nucleotide sequences of the antibodies described herein. For example, “homology” or “identity” or “similarity” refers to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous at that position. A degree of homology between sequences is a function of the number of matching or homologous positions shared by the sequences. For example, the antibodies can have 60%, 70%, 75%, 80%, 85%, 90%, 91%, 92%, 93%, 94%, 95%, 96%, 97%, 98%, 99%, or higher amino acid sequence identity when compared to a specified region or the full length of any one of the antibodies described herein. For example, the antibodies can have 60%, 70%, 75%, 80%, 85%, 90%, 91%, 92%, 93%, 94%, 95%, 96%, 97%, 98%, 99%, or higher nucleic acid identity when compared to a specified region or the full length of any one of the antibodies described herein. Sequence identity or similarity to the nucleic acids and proteins of the present invention can be determined by sequence comparison and/or alignment by methods known in the art, for example, using software programs known in the art, such as those described in Ausubel et al. eds. (2007) Current Protocols in Molecular Biology. For example, sequence comparison algorithms (i.e., BLAST or BLAST 2.0), manual alignment or visual inspection can be utilized to determine percent sequence identity or similarity for the nucleic acids and proteins of the present invention.

Aspects of the invention provide isolated. The term “isolated” as used herein with respect to cells, nucleic acids, such as DNA or RNA, refers to molecules separated from other DNAs or RNAs, respectively, that are present in the natural source of the macromolecule. The term “isolated” can also refer to a nucleic acid or peptide that is substantially free of cellular material, viral material, or culture medium when produced by recombinant DNA techniques, or chemical precursors or other chemicals when chemically synthesized. For example, an “isolated nucleic acid” can include nucleic acid fragments which are not naturally occurring as fragments and would not be found in the natural state. “Isolated” can also refer to cells or polypeptides which are isolated from other cellular proteins or tissues. Isolated polypeptides can include both purified and recombinant polypeptides.

As used herein, an “antibody” or “antigen-binding polypeptide” can refer to a polypeptide or a polypeptide complex that specifically recognizes and binds to an antigen. An antibody can be a whole antibody and any antigen binding fragment or a single chain thereof. For example, “antibody” can include any protein or peptide containing molecule that comprises at least a portion of an immunoglobulin molecule having biological activity of binding to the antigen. Non-limiting examples a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. As used herein, the term “antibody” can refer to an immunoglobulin molecule and immunologically active portions of an immunoglobulin (Ig) molecule, i.e., a molecule that contains an antigen binding site that specifically binds (immunoreacts with) an antigen. By “specifically binds” or “immunoreacts with” is meant that the antibody reacts with one or more antigenic determinants of the desired antigen and does not react with other polypeptides.

The terms “antibody fragment” or “antigen-binding fragment”, as used herein, is a portion of an antibody such as F_{(ab′)2}, F_{(ab)2}, F_{ab}′, F_{ab}, Fv, scFv and the like. Regardless of structure, an antibody fragment binds with the same antigen that is recognized by the intact antibody. The term “antibody fragment” can include aptamers (such as spiegelmers), minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific, human, humanized or chimeric antibodies, single chain antibodies, epitope-binding fragments, e.g., Fab, Fab′ and F(ab′)₂, Fd, Fvs, single-chain Fvs (scFv), single-chain antibodies, dAb (domain antibody), minibodies, disulfide-linked Fvs (sdFv), fragments comprising either a VL or VH domain, fragments produced by a Fab expression library, and anti-idiotypic (anti-Id) antibodies.

A “single-chain variable fragment” or “scFv” refers to a fusion protein of the variable regions of the heavy (V_{H}) and light chains (V_{L}) of immunoglobulins. A single chain Fv (“scFv”) polypeptide molecule is a covalently linked VH:VL heterodimer, which can be expressed from a gene fusion including VH- and VL-encoding genes linked by a peptide-encoding linker. (See Huston et al. (1988) Proc Nat Acad Sci USA 85(16):5879-5883). In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. A number of methods have been described to discern chemical structures for converting the naturally aggregated, but chemically separated, light and heavy polypeptide chains from an antibody V region into an scFv molecule, which will fold into a three-dimensional structure substantially similar to the structure of an antigen-binding site. See, e.g., U.S. Pat. Nos. 5,091,5 13; 5,892,019; 5,132,405; and 4,946,778, each of which are incorporated by reference in their entireties.

Antibody molecules obtained from humans fall into five classes of immunoglobulins: IgG, IgM, IgA, IgE and IgD, which differ from one another by the nature of the heavy chain present in the molecule. Those skilled in the art will appreciate that heavy chains are classified as gamma, mu, alpha, delta, or epsilon (γ, μ, α, δ, ε) with some subclasses among them (e.g., γ1-γ4). Certain classes have subclasses as well, such as IgG₁, IgG₂, IgG₃ and IgG₄ and others. The immunoglobulin subclasses (isotypes) e.g., IgG₁, IgG₂, IgG₃, IgG₄, IgG₅, etc. are well characterized and are known to confer functional specialization. With regard to IgG, a standard immunoglobulin molecule comprises two identical light chain polypeptides of molecular weight approximately 23,000 Daltons, and two identical heavy chain polypeptides of molecular weight 53,000-70,000. The four chains can be joined by disulfide bonds in a “Y” configuration wherein the light chains bracket the heavy chains starting at the mouth of the “Y” and continuing through the variable region. Immunoglobulin or antibody molecules described herein can be of any type (e.g., IgG, IgE, IgM, IgD, IgA, and IgY), class (e.g., IgG1, IgG2, IgG3, IgG4, IgA1 and IgA2) or subclass of an immunoglobulin molecule.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. The variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The term “antigen-binding site,” or “binding portion” can refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

The six CDRs present in each antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domains, the FR regions, show less inter-molecular variability. The framework regions largely adopt a β-sheet conformation and the CDRs form loops which connect, and in some cases form part of, the β-sheet structure. The framework regions act to form a scaffold that provides for positioning the CDRs in correct orientation by inter-chain, non-covalent interactions. The antigen-binding domain formed by the positioned CDRs provides a surface complementary to the epitope on the immunoreactive antigen, which promotes the non-covalent binding of the antibody to its cognate epitope. The amino acids comprising the CDRs and the framework regions, respectively, can be readily identified for a heavy or light chain variable region by one of ordinary skill in the art, since they have been previously defined (See, “Sequences of Proteins of Immunological Interest,” Kabat, E., et al., U.S. Department of Health and Human Services, (1983); and Chothia and Lesk, J Mol. Biol., 196:901-917 (1987)).

Where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

| CDR | Kabat Numbering | Chothia Numbering |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

Kabat et al. defined a numbering system for variable domain sequences that is applicable to any antibody. The skilled artisan can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-H1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10⁻¹⁷ residues; and ends at the next tryptophan residue. CDR-L2 begins at approximately the sixteenth residue after the end of CDR-L1 and includes approximately 7 residues. CDR-L3 begins at approximately the thirty third residue after the end of CDR-L2 (i.e., following a cysteine residue); includes approximately 7-11 residues and ends at the sequence F or W-G-X-G, where X is any amino acid.

As used herein, the term “epitope” can include any protein determinant that can specifically bind to an immunoglobulin, a scFv, or a T-cell receptor. The variable region allows the antibody to selectively recognize and specifically bind epitopes on antigens. For example, the VL domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. Epitopic determinants can consist of chemically active surface groupings of molecules such as amino acids or sugar side chains and can have specific three-dimensional structural characteristics, as well as specific charge characteristics. For example, antibodies can be raised against N- terminal or C-terminal peptides of a polypeptide. More specifically, the antigen-binding site is defined by three CDRs on each of the VH and VL chains (i.e., CDR-H1, CDR-H2, CDR-H3, CDR-L1, CDR-L2 and CDR-L3).

As used herein, the terms “immunological binding,” and “immunological binding properties” can refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361: 186-87 (1993)). The ratio of K_{off}/Kₒₙ allows the cancellation of all parameters not related to affinity, and is equal to the equilibrium binding constant, K_{D}. (See, generally, Davies et al. (1990) Annual Rev Biochem 59:439-473). An antibody of the invention can specifically bind to an epitope when the equilibrium binding constant (K_{D}) is ≤1 μM, ≤10 μM, ≤10 nM, ≤10 pM, or ≤100 pM to about 1 pM, as measured by kinetic assays such as radioligand binding assays or similar assays known to those skilled in the art, such as BIAcore or Octet (BLI). For example, in some embodiments, the K_{D} is between about 1E-12 M and a K_{D} about 1E-11 M. In some embodiments, the K_{D} is between about 1E-11 M and a K_{D} about 1E-10 M. In some embodiments, the K_{D} is between about 1E-10 M and a K_{D} about 1E-9 M. In some embodiments, the K_{D} is between about 1E-9 M and a K_{D} about 1E-8 M. In some embodiments, the K_{D} is between about 1E-8 M and a K_{D} about 1E-7 M. In some embodiments, the K_{D} is between about 1E-7 M and a K_{D} about 1E-6 M. For example, in some embodiments, the K_{D} is about 1E-12 M while in other embodiments the K_{D} is about 1E-11 M. In some embodiments, the K_{D} is about 1E-10 M while in other embodiments the K_{D} is about 1E-9 M. In some embodiments, the K_{D} is about 1E-8 M while in other embodiments the K_{D} is about 1E-7 M. In some embodiments, the K_{D} is about 1E-6 M while in other embodiments the K_{D} is about 1E-5 M. In some embodiments, for example, the K_{D} is about 3 E-11 M, while in other embodiments the K_{D} is about 3E-12 M. In some embodiments, the K_{D} is about 6E-11 M. “Specifically binds” or “has specificity to,” can refer to an antibody that binds to an epitope via its antigen-binding domain, and that the binding entails some complementarity between the antigen-binding domain and the epitope. For example, an antibody is said to “specifically bind” to an epitope when it binds to that epitope, via its antigen-binding domain more readily than it would bind to a random, unrelated epitope.

For example, the antibody can be monovalent or multivalent (e.g., bivalent), and can comprise a single or double chain. Functionally, the binding affinity of the antibody is within the range of 10⁻⁵ M to 10⁻¹² M. For example, the binding affinity of the antibody is from 10⁻⁶ M to 10⁻¹² M, from 10⁻⁷ M to 10⁻¹² M, from 10⁻⁸ M to 10⁻¹² M, from 10⁻⁹ M to 10⁻¹² M, from 10⁻⁵ M to 10⁻¹¹ M, from 10⁻⁶ M to 10⁻¹¹ M, from 10⁻⁷ M to 10⁻¹¹ M, from 10⁻⁸ M to 10⁻¹¹ M, from 10⁻⁹ M to 10⁻¹¹ M, from 10⁻¹⁰ M to 10⁻¹¹ M, from 10⁻⁵ M to 10⁻¹⁰M, from 10⁻ M to 10⁻¹⁰ M, from 10⁻⁷ M to 10⁻¹⁰ M, from 10⁻⁸ M to 10⁻¹⁰ M, from 10⁻⁹ M to 10⁻¹⁰ M, from 10⁻⁵ M to 10⁻⁹ M, from 10⁻⁶ M to 10⁻⁹ M, from 10⁻⁷ M to 10⁻⁹ M, from 10⁻⁸ M to 10⁻⁹ M, from 10⁻⁵ M to 10⁻⁸ M, from 10⁻⁶ M to 10⁻⁸ M, from 10⁻⁷ M to 10⁻⁸ M, from 10⁻⁵ M to 10⁻⁷ M, from 10⁻⁶ M to 10⁻⁷ M, or from 10⁻⁵ M to 10⁻⁶ M.

A protein, or a derivative, fragment, analog, homolog or ortholog thereof, can be utilized as an immunogen in the generation of the antibodies. A protein or a derivative, fragment, analog, homolog, or ortholog thereof, coupled to a proteoliposome can be utilized as an immunogen in the generation of antibodies that immunospecifically bind these protein components.

Those skilled in the art can determine, without undue experimentation, if a human monoclonal antibody has the same specificity as a human monoclonal antibody of the invention by ascertaining whether the former prevents the latter from binding to its immunogen or target. For example, if the human monoclonal antibody being tested competes with the human monoclonal antibody of the invention, as shown by a decrease in binding by the human monoclonal antibody of the invention, then the two monoclonal antibodies bind to the same, or to a closely related, epitope.

Another way to determine whether a human monoclonal antibody has the specificity of a human monoclonal antibody of the invention is to pre-incubate the human monoclonal antibody of the invention with the immunogen or target, with which it is normally reactive, and then add the human monoclonal antibody being tested to determine if the human monoclonal antibody being tested is inhibited in its ability to bind the target. If the human monoclonal antibody being tested is inhibited then, in all likelihood, it has the same, or functionally equivalent, epitopic specificity as the monoclonal antibody of the invention. Screening of human monoclonal antibodies of the invention can be also carried out by utilizing the immunogen/target and determining whether the test monoclonal antibody is able to bind or neutralize the target.

Various procedures known within the art can be used for the production of polyclonal or monoclonal antibodies directed against a protein of the invention, or against derivatives, fragments, analogs homologs or orthologs thereof (See, for example, Antibodies: A Laboratory Manual, Harlow E, and Lane D, 1988, Cold Spring Harbor Laboratory Press, Cold Spring Harbor, NY, incorporated herein by reference).

Antibodies can be purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, can be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

The term “monoclonal antibody” or “mAb” or “Mab” or “monoclonal antibody composition”, as used herein, can refer to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site that immunoreacts with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, can be immunized with an immunizing agent to elicit lymphocytes that produce or can produce antibodies that will specifically bind to the immunizing agent. Alternatively, the lymphocytes can be immunized in vitro.

### Multisnecific Antibodies (Bisnecific, Trisnecific)

Multispecific antibodies are antibodies that can recognize two or more different antigens. For example, a bi-specific antibody (bsAb) is an antibody comprising two variable domains or scFv units such that the resulting antibody recognizes two different antigens. For example, a trispecific antibody (tsAb) is an antibody comprising two variable domains or scFv units such that the resulting antibody recognizes three different antigens. This invention provides for multispecific antibodies, such as bi-specific and trispecific antibodies, that recognize Ly49/iKIR, CD8 and/or CD8 Treg TCRs. In some embodiments, the bispecific and trispecific antibodies can include fusion proteins. For example, the fusion protein can include an antibody comprising a variable domain or scFv unit and a ligand or antigen and/or a third ligand or antigen as described herein such that the resulting antibody recognizes an antigen and binds to the ligand-specific receptor. In some embodiments, the fusion protein further comprises a constant region, and/or a linker as described herein.

Different formats of bispecific or trispecific antibodies are also provided herein. In some embodiments, each of the first antigen-specific fragment, the second antigen-specific fragment and/or the third antigen-specific fragment is each independently selected from a Fab fragment, a single-chain variable fragment (scFv), or a single-domain antibody. In some embodiments, the bispecific or trispecific antibody further includes a Fc fragment (e.g., as described in PCT/US2015/021529 and PCT/US2019/023382, each of which are incorporated by reference in their entireties). A bispecific or trispecific antibody of the invention can comprise a heavy chain and a light chain combination or scFv antibodies as described herein.

Multispecific antibodies (e.g., bispecific antibodies and trispecific antibodies) of the invention can be constructed using methods known art. In some embodiments, the bi-specific antibody is a single polypeptide wherein the two scFv fragments are joined by a long linker polypeptide, of sufficient length to allow intramolecular association between the two scFv units to form an antibody. In other embodiments, the bi-specific antibody is more than one polypeptide linked by covalent or non-covalent bonds. In some embodiments, the amino acid linker depicted herein (GGGGSGGGGS; “(G₄S)₂”), can be generated with a longer G₄S linker to improve flexibility. For example, the linker can also be “(G₄S)₃” (e.g., GGGGSGGGGSGGGGS); “(G₄S)₄” (e.g., GGGGSGGGGSGGGGSGGGGS); “(G₄S)₅” (e.g., GGGGSGGGGSGGGGSGGGGSGGGGS); “(G₄S)₆” (e.g., GGGGSGGGGSGGGGSGGGGSGGGGSGGGGS); “(G₄S)₇” (e.g., GGGGSGGGGSGGGGSGGGGSGGGGSGGGGSGGGGS); and the like. For example, use of the (G₄S)₅ linker can provide more flexibility to a ligand described herein and can improve expression. In some embodiments, the linker can also be (GS)ₙ, (GGS)ₙ, (GGGS)ₙ, (GGSG)ₙ, (GGSGG)ₙ, or (GGGGS)ₙ, wherein n is 1, 2, 3, 4, 5, 6, 7, 8, 9, or 10. Non-limiting examples of linkers known to those skilled in the art that can be used to construct the fusions described herein can be found in U.S. Pat. No. 9,708,412; U.S. Patent Application Publication Nos. US 20180134789 and US 20200148771; and PCT Publication No. WO2019051122 (each of which are incorporated by reference in their entireties).

In another embodiment, the multispecific antibodies can be constructed using the “knob into hole” method (Ridgway et al, Protein Eng 7:617-621 (1996)). In this method, the Ig heavy chains of the two different variable domains are reduced to selectively break the heavy chain pairing while retaining the heavy-light chain pairing. The two heavy-light chain heterodimers that recognize two different antigens/ligands or three different antigens/ligands are mixed to promote heteroligation pairing, which is mediated through the engineered “knob into holes” of the CH3 domains.

In another embodiment the multispecific antibodies can be constructed through exchange of heavy-light chain dimers from two or more different antibodies to generate a hybrid antibody where the first heavy-light chain dimer recognizes a first antigen and the second heavy-light chain dimer recognizes a second antigen and/or third antigen. The mechanism for heavy-light chain dimer is similar to the formation of human IgG₄, which also functions as a bispecific molecule. Dimerization of IgG heavy chains is driven by intramolecular force, such as the pairing the CH3 domain of each heavy chain and disulfide bridges. Presence of a specific amino acid in the CH3 domain (R409) has been shown to promote dimer exchange and construction of the IgG₄ molecules. Heavy chain pairing is also stabilized further by interheavy chain disulfide bridges in the hinge region of the antibody. Specifically, in IgG₄, the hinge region contains the amino acid sequence Cys-Pro-Ser-Cys (in comparison to the stable IgG1 hinge region which contains the sequence Cys-Pro-Pro-Cys) at amino acids 226-230. This sequence difference of Serine at position 229 has been linked to the tendency of IgG₄ to form intrachain disulfides in the hinge region (Van der Neut Kolfschoten, M. et al, 2007, Science 317: 1554-1557 and Labrijn, A. F. et al, 2011, Journal of Immunol 187:3238-3246).

The multispecific antibodies of the invention can be created through introduction of the R409 residue in the CH3 domain and the Cys-Pro-Ser-Cys sequence in the hinge region of antibodies that recognize the first or a second and/or third antigen, so that the heavy-light chain dimers exchange to produce an antibody molecule with one heavy-light chain dimer recognizing the first and the second heavy-light chain dimer recognizing a second and/or third antigen, wherein the second and/or third antigen (or ligand) is any antigen (or ligand) disclosed herein. Known IgG₄ molecules can also be altered such that the heavy and light chains recognize the first or a second and/or third antigen, as disclosed herein. Use of this method for constructing the multispecific antibodies of the invention can be beneficial due to the intrinsic characteristic of IgG₄ molecules wherein the Fc region differs from other IgG subtypes in that it interacts poorly with effector systems of the immune response, such as complement and Fc receptors expressed by certain white blood cells. This specific property makes these IgG₄-based multispecific antibodies attractive for therapeutic applications, in which the antibody is required to bind the target(s) and functionally alter the signaling pathways associated with the target(s), however not trigger effector activities.

The multispecific antibodies described herein can be engineered with a non-depleting heavy chain isotype, such as IgG1-LALA or stabilized IgG₄ or one of the other non-depleting variants. In some embodiments, mutations are introduced to the constant regions of the bsAb such that the antibody dependent cell-mediated cytotoxicity (ADCC) activity of the bsAb is altered. For example, the mutation is a LALA mutation in the CH2 domain. In one aspect, the multispecific antibody contains mutations on one scFv unit of the heterodimeric multispecific antibody, which reduces the ADCC activity. In another aspect, the multispecific antibody contains mutations on both chains of the heterodimeric multispecific antibody, which completely ablates the ADCC activity. For example, the mutations introduced in one or both scFv units of the multispecific antibody are LALA mutations in the CH2 domain. These multispecific antibodies with variable ADCC activity can be optimized such that the multi-specific antibodies exhibit maximal selective killing towards cells that express one antigen that is recognized by the multispecific antibody, however, exhibits minimal killing towards the second antigen that is recognized by the multispecific antibody.

The multispecific antibodies (e.g., bispecific antibodies) described herein can be engineered as modular tetrameric bispecific antibodies (tBsAb). See, for example, WO 2018/071913, which is incorporated by reference herein in its entirety. For example, the tetravalent antibody can be a dimer of a bispecific scFv fragment including a first binding site for a first antigen, and a second binding site for a second antigen. In embodiments, the first antibody can be the first binding site for a first antigen. In embodiments, the second antibody can be the second binding site for a second antigen. The two binding sites can be joined together via a linker domain. In embodiments, the scFv fragment is a tandem scFv, the linker domain includes an immunoglobulin hinge region (e.g., an IgG1, an IgG₂, an IgG₃, or an IgG₄ hinge region) amino acid sequence. In embodiments, the immunoglobulin hinge region amino acid sequence can be flanked by a flexible linker amino acid sequence, e.g., having the linker amino acid sequence (GGGS)ₓ₁₋₆, (GGGGS)ₓ₁₋₆, or GSAGSAAGSGEF. In embodiments, the linker domain includes at least a portion of an immunoglobulin Fc domain, e.g., an IgG1, an IgG₂, an IgG₃, or an IgG₄ Fc domain. In embodiments, the at least a portion of the immunoglobulin Fc domain does not include a CH2 domain. In embodiments, the at least a portion of the immunoglobulin Fc domain can be a CH2 domain. An exemplary CH2 domain amino acid sequence includes APELLGGPDVFLF. The Fc domain can be linked to the C-terminus of an immunoglobulin hinge region (e.g., an IgG1, an IgG₂, an IgG₃, or an IgG₄ hinge region) amino acid sequence. The linker domain can include a flexible linker amino acid sequence (e.g., (GGGS)ₓi-6, (GGGGS)ₓi-6, or GSAGSAAGSGEF) at one terminus or at both termini.

### Antibody-Mediated Rejection, Tumor Surveillance

In some embodiments, mobilizing CD8 Treg cells in a mammal using the peptide agonists or antibodies that mobilize or activate CD8 Treg cells can be used to an organ transplant patient or a patient with an autoimmune disease.

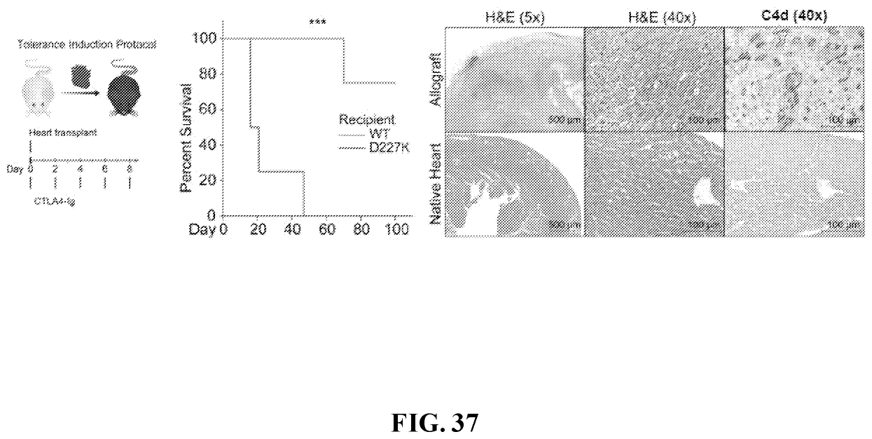

Regarding treatment of organ transplant patients, in various embodiments, the reagents and methods described herein can be used to treat patients with hyperacute rejection, acute rejection, or chronic rejection. In embodiments, the reagents and methods can be used to treat antibody-mediated rejection (AMR; see FIGS. 37-41, 60A-F, 77, 83A-F, 84A-H) in a patient. In embodiments, the reagents and methods can be used to treat cell-based allograft rejection (e.g., cytotoxic T cell-based rejection) and antibody-mediated allograft rejection. In general, the treatments diminish humoral and/or cellular rejection of the allograft.

In various embodiments, the allograft rejections that can be treated using the reagents and methods described herein include any type of transplant. In embodiments, patients having at least the following allograft transplants can be treated: heart, kidney lung, liver, pancreas, cornea, trachea, skin, vacscular tissues, stem cell, bone and others.

Regarding autoimmune diseases, the reagents and methods described herein can be used to treat patients having all different types of autoimmune diseases or disorders. In some embodiments, patients having at least systemic lupus erythematosus (SLE), multiple sclerosis (MS), type 1 diabetes (DM1; insulin dependent diabetes mellitus or IDDM), rheumatoid arthritis, psoriasis or psoriatic arthritis, inflammatory bowel disease, Addison's disease, Graves' disease, Sjögren's syndrome, Hashimoto's thyroiditis, myasthenia gravis, autoimmune vasculitis, pernicious anemia, celiac disease, as well as others.

The reagents and methods described herein can be used to treat patients with inflammation or inflammatory disorders.

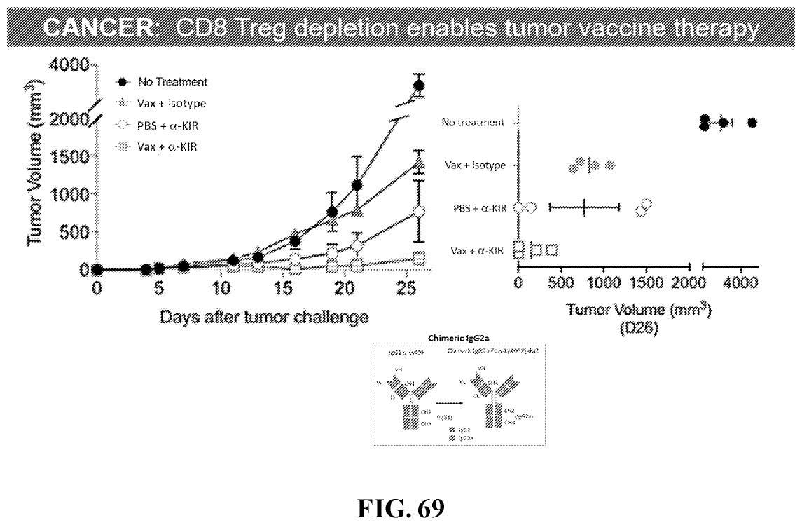

In some embodiments, suppressing and/or killing CD8 Treg cells in a mammal using antibodies that bind to CD8 Treg cells or molecules of CD8 Treg cells can increase anti-tumor activity in the mammal. The antibodies can be used to treat tumors or cancer in a mammal (see FIGS. 69, 71, 89A-D, 91A-C). The antibodies used in these embodiments are described in the sections of this application titled “Antibodies to CD8 Treg” and “Multispecific Antibodies.”

Generally, depletion of CD8 Treg can increase CD4 T cell activity and anti-tumor activity within a mammalian organism.

Generally, the reagents and methods disclosed herein can be used to treat any cancer. A nonlimiting list of cancers for which the reagents and methods disclosed herein include bladder, breast, colon and rectal, endometrial, kidney, leukemia, liver, lung, lymnphoma (e.g., Non-Hodgkin lymphoma), melanoma, pancreatic, prostate, thyroid, and others.

In some embodiments, the reagents and methods disclosed herein for treating tumors or cancer in a mammal (e.g., human) can be combined with other types of anti-cancer therapy. In some embodiments, the treatments disclosed herein can be used in combination with a therapeutic cancer vaccine. In some embodiments, the treatments disclosed herein can be used in combination with an immune checkpoint inhibitor or checkpoint inhibitor. In some embodiments, the immune checkpoint inhibitor can include PD-L1 inhibitors.

### Therapeutic Preparations

Aspects of the invention are drawn towards therapeutic preparations. As used herein, the term “therapeutic preparation” can refer to any compound or composition (e.g., including cells) that can be used or administered for therapeutic effects. As used herein, the term “therapeutic effects” can refer to effects sufficient to result in amelioration of symptoms, e.g., treatment, healing, prevention or amelioration of the relevant medical condition, or an increase in rate of treatment, healing, prevention or amelioration of such conditions. In some embodiments, therapeutic effect may refer to those resulting from treatment of cancer in a subject of patient. Herein, a therapeutic preparation can include a pharmaceutical composition.

Pharmaceutical compositions disclosed herein can include therapeutically effective amounts of any of the CD8 Treg stimulators disclosed herein (see section titled “CD8 Treg Agonists”). In some embodiments, the CD8 Treg stimulators can include peptide/polypeptide agonists/superagonists as discussed in that section. In some embodiments, the CD8 Treg stimulators can include antibodies that mobilize or activate CD8 Treg cells (see sections titled “Antibodies to CD8 Treg” and “Multispecific Antibodies”). In some embodiments, the pharmaceutical compositions can include combinations of the peptide agonists and the stimulator antibodies. These pharmaceutical compositions can include a pharmaceutically acceptable carrier. In some embodiments, the peptides and/or antibodies of these compositions can be conjugated to a lipophilic albumin binding tail conjugate.

Generally, these pharmaceutical compositions, that mobilize CD8 Treg cells, can suppress CD4 T cell activity in a mammal to which the composition is administered. These pharmaceutical compositions can decrease expression of T follicular cells (Tfh), germline center B cells, antibody generation, or combinations thereof. These pharmaceutical compositions can decrease production of donor-specific antibodies and/or graft tissue injury. In embodiments, these pharmaceutical compositions can be used to treat organ transplant patients to prevent or decrease the probability that a transplanted organ will be rejected. In embodiments, these pharmaceutical compositions can be used to treat patients that have various autoimmune diseases.

Pharmaceutical compositions disclosed herein can include therapeutically effective amounts of any of the molecules disclosed herein that deplete CD8 Treg cells in a mammal. In some embodiments, these CD8 Treg depleters can be antibodies (see sections of this application titled “Antibodies to CD8 Treg” and “Multispecific Antibodies”). These pharmaceutical compositions can include a pharmaceutically acceptable carrier. In some embodiments, the peptides and/or antibodies of these compositions can be conjugated to a lipophilic albumin binding tail conjugate.

Generally, these pharmaceutical compositions, that deplete CD8 Treg cells, can increase CD4 T cell activity in a mammal to which the composition is administered. These pharmaceutical compositions can increase expression of T follicular cells (Tfh), germline center B cells, antibody generation, or combinations thereof. These pharmaceutical compositions can increase production of donor-specific antibodies. In embodiments, these pharmaceutical compositions can be used to treat tumors or cancer in patients.

Embodiments as described herein can be administered to a subject in the form of a pharmaceutical composition or therapeutic preparation prepared for the intended route of administration. Such compositions and preparations can comprise, for example, the active ingredient(s) and a pharmaceutically acceptable carrier. Such compositions and preparations can be in a form adapted to oral, subcutaneous, parenteral (such as, intravenous, intraperitoneal), intramuscular, rectal, epidural, intratracheal, intranasal, dermal, vaginal, buccal, ocularly, or pulmonary administration, such as in a form adapted for administration by a peripheral route or is suitable for oral administration or suitable for parenteral administration. Other routes of administration are subcutaneous, intraperitoneal and intravenous, and such compositions can be prepared in a manner well-known to the person skilled in the art, e.g., as generally described in “Remington's Pharmaceutical Sciences”, 17. Ed. Alfonso R. Gennaro (Ed.), Mark Publishing Company, Easton, Pa., U.S.A., 1985 and more recent editions and in the monographs in the “Drugs and the Pharmaceutical Sciences” series, Marcel Dekker. The compositions and preparations can appear in conventional forms, for example, solutions and suspensions for injection, capsules and tablets, in the form of enteric formulations, e.g., as disclosed in U.S. Pat. No. 5,350,741, and for oral administration.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Oral formula of the drug can be administered once a day, twice a day, three times a day, or four times a day, for example, depending on the half-life of the drug.

Pharmaceutically compatible binding agents, and/or adjuvant materials can be included as part of the composition administered to a subject. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In embodiments, administering can comprise the placement of a pharmaceutical composition into a subject by a method or route which results in at least partial localization of the composition at a desired site such that desired effect is produced.

For example, the pharmaceutical composition can be administered by bolus injection or by infusion. A bolus injection can refer to a route of administration in which a syrine is connected to the IV access device and the medication is injected directly into the subject. The term “infusion” can refer to an intravascular injection.

Embodiments as described herein can be administered to a subject one time (e.g., as a single injection, bolus, or deposition). Alternatively, administration can be once or twice daily to a subject for a period of time, such as from about 2 weeks to about 28 days. Administration can continue for up to one year. In embodments, administration can continue for the life of the subject. It can also be administered once or twice daily to a subject for period of 1, 2, 3, 4, 5, 6, 7, 8, 9, 10, 11, 12 times per year, or a combination thereof.

In embodiments, compositions as described herein can be administered to a subject chronically. “Chronic administration” can refer to administration in a continuous manner, such as to maintain the therapeutic effect (activity) over a prolonged period of time.

The pharmaceutical or therapeutic carrier or diluent employed can be a conventional solid or liquid carrier. Examples of solid carriers are lactose, terra alba, sucrose, cyclodextrin, talc, gelatin, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene and water. Similarly, the carrier or diluent can include any sustained release material known in the art, such as glyceryl monostearate or glyceryl distearate, alone or mixed with a wax.

When a solid carrier is used for oral administration, the preparation can be tabletted, placed in a hard gelatin capsule in powder or pellet form or it can be in the form of a troche or lozenge. The amount of solid carrier will vary widely but can be from about 25 mg to about 1 g.

When a liquid carrier is used, the preparation can be in the form of a syrup, emulsion, soft gelatin capsule or sterile injectable liquid such as an aqueous or non-aqueous liquid suspension or solution.

The composition and/or preparation can also be in a form suited for local or systemic injection or infusion and can, as such, be formulated with sterile water or an isotonic saline or glucose solution. The compositions can be in a form adapted for peripheral administration only, with the exception of centrally administrable forms. The compositions and/or preparations can be in a form adapted for central administration.

The compositions and/or preparations can be sterilized by conventional sterilization techniques which are well known in the art. The resulting aqueous solutions can be packaged for use or filtered under aseptic conditions and lyophilized, the lyophilized preparation being combined with the sterile aqueous solution prior to administration. The compositions and/or preparations can contain pharmaceutically and/or therapeutically acceptable auxiliary substances as required to approximate physiological conditions, such as buffering agents, tonicity adjusting agents and the like, for instance sodium acetate, sodium lactate, sodium chloride, potassium chloride, calcium chloride, etc.

### Vaccine Compositions

Herein, vaccine compositions refer to therapeutically effective amounts of CD8 Treg agonists, including peptide/polypeptide agonists/superagonists. Vaccine compositions can include any of the peptide/polypeptide agonists/superagonists disclosed in the section of this application titled “CD8 Treg Agonists.” In some embodiments, the vaccine compositions can include pharmaceutically acceptable carriers, diluents or excipients. In some embodiments, the peptide/polypeptide agonists/superagonists in the vaccine compositions are conjugated to a carrier protein or proteins. In some embodiments, the carrier protein can include a lipophilic albumin binding tail conjugate. In some embodiments, the lipophilic albumin binding tail conjugate can include 1, 2-Distearoyl-sn-glycero-3-phosphoethanolamine-Poly(ethylene glycol) (DSPE- PEG).

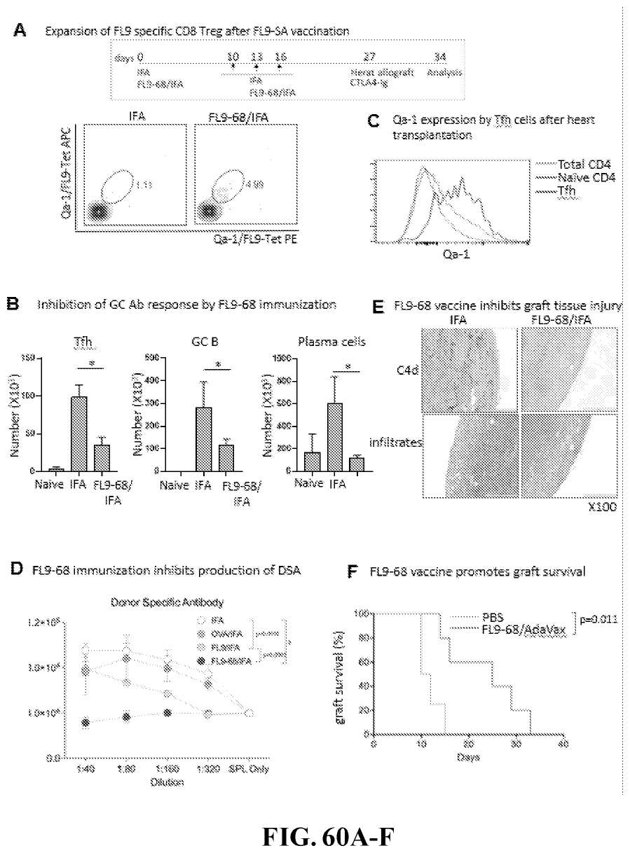

Example data on use of vaccine compositions are shown in FIGS. 60A-F and 61.

### Methods

The methods disclosed herein relate to administration of the disclosed peptide/polypeptide agonists/superagonists, antibodies, combinations thereof, and pharmaceutical compositions of the same, to mammals (e.g., humans, mice) to treat or prevent various conditions.

In some embodiments, the methods include administering a CD8 Treg stimulator to a mammal. The methods for administering a CD8 T cell stimulator can include administering a peptide/polypeptide agonist/superagonist to a mammal. The peptide/polypeptide agonist/superagonist, or combinations of different of the agonists/superagonists, are those described herein (see section titled “CD8 Treg Agonists”).

The methods for administering a CD8 T cell stimulator can include administering an antibody that binds to a CD8 Treg cell to a mammal. These antibodies and multispecific antibodies are described herein (see the sections titled “Antibodies to CD8 Treg” and “Multispecific Antibodies”).

Administration of these CD8 Treg stimulators can mobilize CD8 Treg cells in the mammal and suppress or decrease CD4 T cells and activity in the mammal. In some embodiments, these CD8 Treg stimulators can be administered to mammals receiving an organ transplant. The administration can diminish humoral- and/or cellular-based rejection of the transplanted organ in the mammal. In some embodiments, these CD8 Treg stimulators can be administered to mammals that have an autoimmune disease or disorder.

Administration of the CD8 Treg cell stimulators can be used to increase effector CD8 Treg cells, treat an autoimmune disease or condition, and/or treat or prevent rejection of a transplanted organ (e.g., antibody-mediated rejection) in a mammal.

In some embodiments, the methods include administering a CD8 Treg depleters to a mammal. The methods for administering a CD8 T cell depleter can include administering an antibody to the mammal that depletes CD8 Treg (see the sections herein titled “Antibodies to CD8 Treg” and “Multispecific Antibodies”).

Administration of the CD8 Treg depleters (e.g., antibodies and/or multispecific antibodies) can increase CD4 T cell activity in a mammal. In some embodiments, these CD8 Treg cell depleters can be used to treat cancer in a mammal. In embodiments, administration of these antibodies can increase anti-tumor activity in the mammal.

Administration of the CD8 Treg cell depleters can be used to decrease effector CD8 Treg cells and/or treat cancer in a mammal.

Also disclosed are methods of screening for an autoimmune disorder in a mammal by detecting any of the TCRs described in the section of this application titled “CD8 Treg Cells.” These diagnostic methods can be used prior to treating a mammal having an autoimmune disease, or prior to treating a mammal having an autoimmune disease with any of the CD8 Treg stimulators disclosed herein.

Also disclosed are methods for screening an antibody for reactivity to CD8 Treg cells by contacting an antibody with a TCR from a CD8 Treg cell, or with a CD8 Treg cell and detecting binding of the antibody to the TCR and/or CD8 Treg cell.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

FIG. 1A-B CD8 Treg recognize chronically activated CD4 cells that express Qa-1-FL9 peptide on the surface as a result of diminished ERAAP activity. CD8 Treg recognition of target CD4 cells results in cytolysis of these cells and therefore inhibition of their expansion. CD8 Treg recognition of target CD4 cells involves molecular interactions that deliver either stimulatory or inhibitory signals. TCR-Qa-1/peptide and NKG2D-NKG2DL interactions activate CD8 Treg, while NKG2A/CD94-Qa-1/Qdm and Ly49F-ligand interactions inhibit CD8 Treg function.

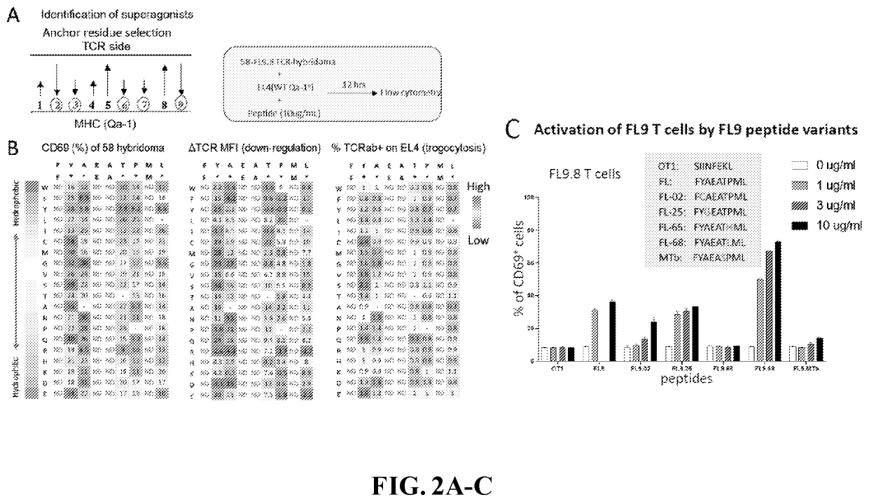

FIG. 2A-C Screening of FL9 peptide variants for their FL9 T cell stimulatory ability led to identification of FL9 superagonist peptides (e.g., FL9-68) that display a superior ability to activate FL9 T cells.

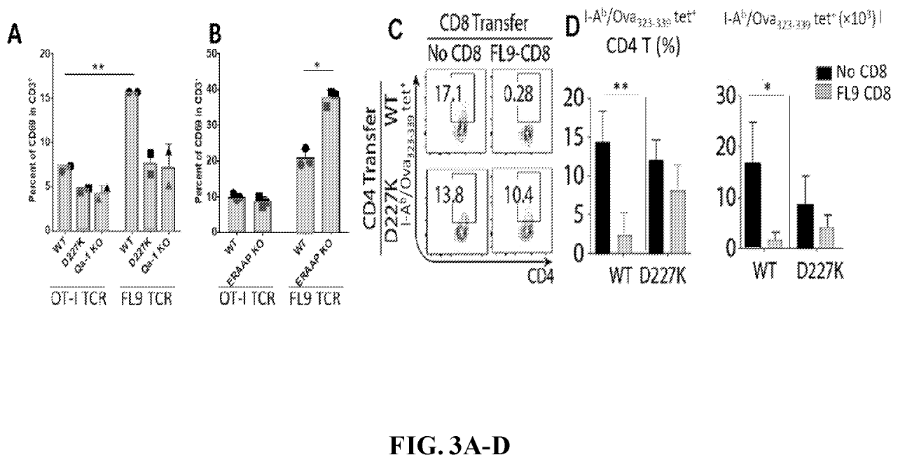

FIG. 3A-D FL9 T cells can be activated by chronically activated CD4 cells that present FL9 peptide in the context of Qa-1. The peptide presented on Qa-1 can be generated in the context of defective ERAAP function or ERAAP deficiency. Recognition of Qa-1-FL9 by FL9 T cells in vivo leads to elimination of Qa-1-FL9⁺ activated CD4 cells (Ova-activated OT-II cells in this experiment).

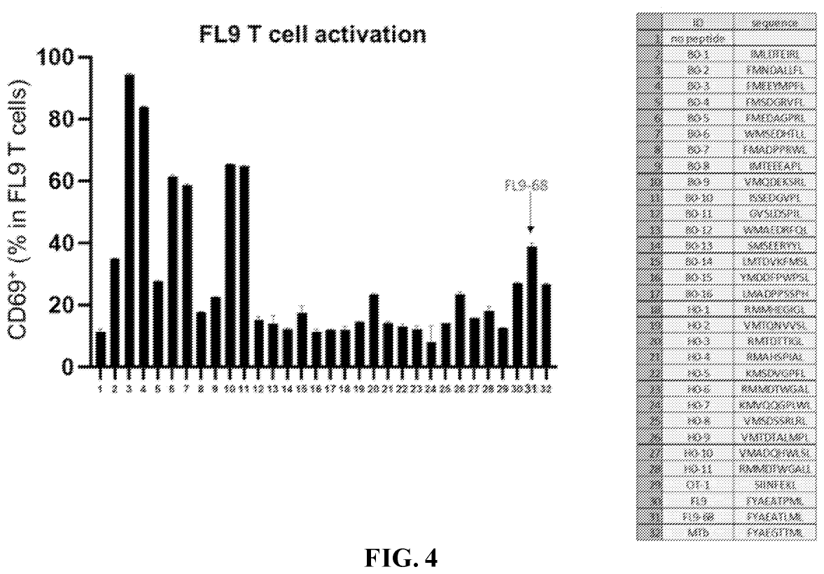

FIG. 4 Screen of a Qa-1 yeast peptide library for superagonist peptides for FL9 T cells resulted in identification of multiple peptides (peptides 3,4,10,11) that display superior ability to stimulate FL9 T cells than FL9-68.

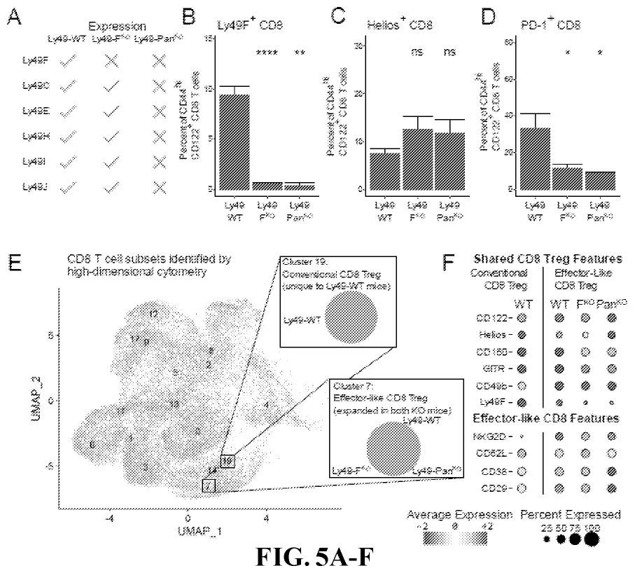

FIG. 5A-F By generating Ly49F and pan Ly49 KO mice, the contribution of the Ly49 receptor to the CD8 Treg phenotype was assessed. Comparison of key markers between WT and Ly49F or pan Ly49 KO CD8 Treg showed that Ly49 deficiency results in differentiation of CD8 Treg with increased activated phenotype.

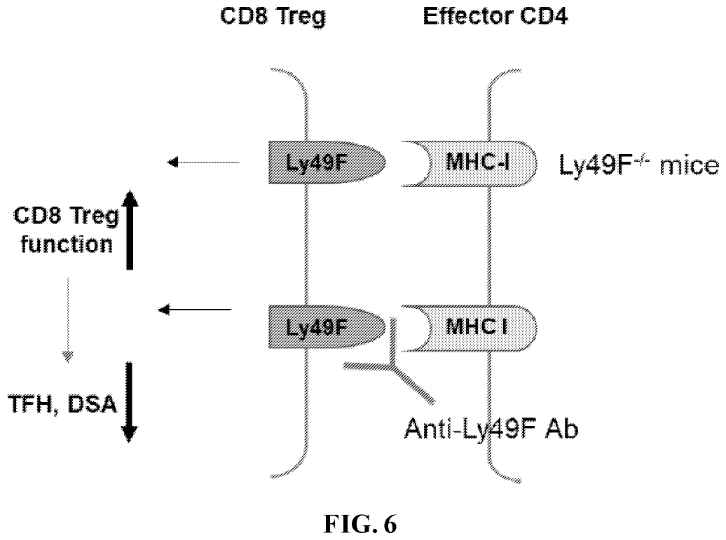

FIG. 6 CD8 Treg function may be manipulated by blocking inhibitory Ly49 signaling using antibodies, as was observed in Ly49 deficient CD8 Treg.

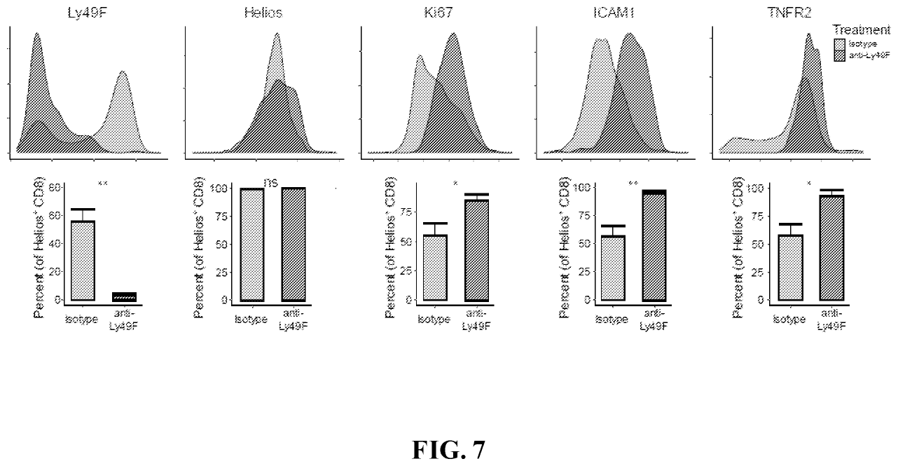

FIG. 7 Ab mediated blocking of Ly49 signaling in CD8 Treg results in activation of CD8 Treg as evidenced by increased proliferation and upregulation of ICAM1 and TNFR2.

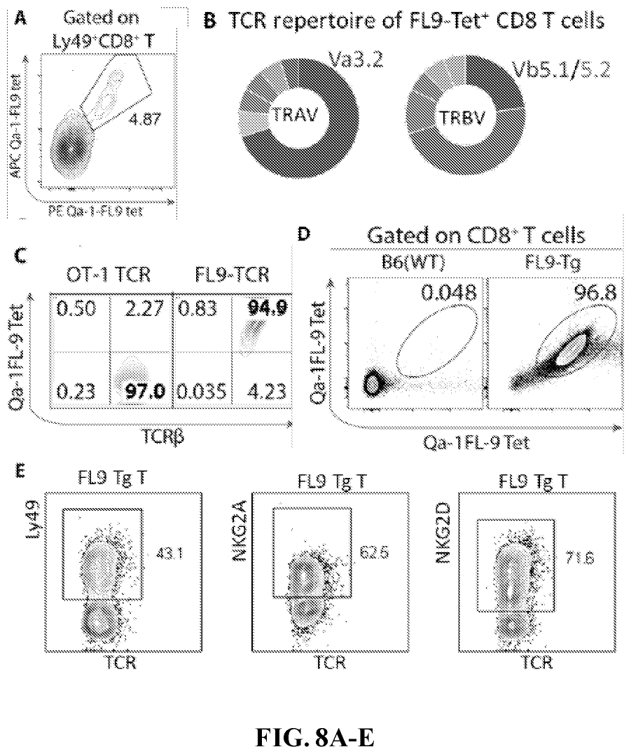

FIG. 8A-E FL9 TCR were identified by sequencing TCRs from Ly49⁺ Qa-1-FL9 tet+CD8 T cells. FL9 TCR Tg mice were generated to faithfully express TCRs specific for Qa-1-FL9 peptide. FL9 TCR Tg CD8 T cells express significant levels of Ly49, NKG2A and NKG2D, reflecting the acquisition of a CD8 Treg phenotype.

### Example 2

CD8 Treg regulate immune responses against pathogens and self-antigens by eliminating chronically activated CD4 cells that upregulate Qa-1/HLA-E on their surface. Recognition of Qa-1-self-peptide on target cells by CD8 Treg can suppress pathogenic CD4 cells, but CD8⁺ Treg expansion and mobilization are constrained by molecular mechanisms that constrain excessive or inappropriate CD8 Treg activation. We have developed strategies that allow both antigen-specific and antigen-nonspecific therapeutic mobilization of CD8 Treg in the context of autoimmune disease and cancer.

### Mobilization of CD8 Treg Response

Peptide superagonists: activation of CD8⁺ Treg in autoimmune disease.

CD8⁺ Treg recognize peptides presented by target CD4 cells in the context of Qa-1. We have identified TCRs that recognize Qa-1 and the FL9 self-peptide. This Qa-1-FL9 complex is expressed by a significant fraction of activated CD4⁺ cells. Activation of CD8+Treg by the FL9-Qa-1/HLA-E complex expressed by target CD4⁺ cells, particularly on Tfh cells, results in inhibition of antibody responses. Mobilization of Qa-1 restricted CD8⁺ Treg uses stimulation with agonistic peptides for robust activation of these regulatory cells. HLA-E also presents the same FL9 amino acid sequence and agonist peptides derived from the FL9 sequence can activate and expand human CD8⁺ Treg. Here, we describe identification of superagonist variants of self-peptides that are engineered to express potent CD8⁺ Treg stimulatory activity in association with Qa-1b and HLA-E. In vitro studies showed that superagonist FL9 peptide promoted proliferation of FL9 T cells leading to enhanced killing of activated CD4 cells. In addition, after immunization with self-antigen (MOG), the formation of Tfh and GC B cells and antibody generation was suppressed following adoptive transfer of FL9 T cells. Vaccination with FL9 superagonist peptides results in efficient mobilization of CD8⁺ Treg and inhibition of antibody-mediated immune responses.

### Anti-Costimulatory Receptor (Ly49F; KIR) Mediated Modulation of CD8 Treg Activity.

1. Autoimmune disease: CD8⁺ Treg express an inhibitory Ly49 receptor (Ly49F, inhibitory KIR in human). The Ly49 receptors are type II C-type lectin-like membrane glycoproteins that recognize class I major histocompatibility complex-I (MHC-I) and MHC-I-like proteins on normal as well as altered cells.

Mouse: Although several Ly49 receptors are expressed by NK cells, we have shown that the murine inhibitory Ly49F receptor is selectively expressed by CD8 Treg and not by conventional CD8⁺ T cells nor NK cells. Studies using a genetic model of Ly49F KO mice showed that Ly49F deficient CD8 Treg display an activated and effector phenotype. Preliminary data show that formation of GC B cells after immunization with antigen (NP-Ova) is reduced in Ly49F KO compared to WT mice, indicating that Ab generation can be inhibited in the absence of Ly49F on CD8 Treg secondary to enhanced CD8 Treg mediated immune suppression. Based on these studies, and while not wishing to be held to a mechanism of action, blockade of Ly49/KIR expression may enhance CD8⁺ Treg mobilization and increase suppressive function by releasing the brakes on this regulatory lineage.

Human: Killer-cell immunoglobulin-like receptors (KIR) are the functional homolog of Ly49F, and human CD8 Treg express inhibitory KIRs (e.g., KIR3DL1, KIR2DL3) on the surface. In vitro suppression assays showed that KIR⁺ CD8 T cells suppress expansion of human Tfh (CXCR5⁺ CD4) cells in vitro. Without wishing to be bound by theory, releasing the brakes on the human CD8 Treg response using anti-iKIR antibodies can enhance their suppressive activity. This approach can be used to suppress expansion of pathogenic CD4 cells during robust inflammation, autoimmunity and infection.

### 2. Cancer

Anti-Ly49F (anti-iKIR Ab in human) Ab mediated depletion of CD8 Treg activity.

We have found that Qa-1 mutant mice (B6.D227K mice), which lack CD8 Treg activity, develop enhanced anti-tumor immune responses to B16 melanoma after GVAX immunization. Reduced tumor growth in B6.D227K mice is associated with enhanced expansion of Tfh cells, GC B cells and high titers of antitumor autoantibodies. A recent retrospective analysis of human cancers showed that anti-PD1 Ab treatment of mNSCLC patients that resulted in increased levels of serum autoantibodies were predictive of positive outcomes (Giannicola R Mol Cli Onc 2019). These studies indicate that depletion of CD8 Treg can enhance anti-tumor immune response by promoting tumor associated Ab generation.

We have developed strategies to selectively deplete CD8 Treg using anti-Ly49F Abs:

A) Studies can be performed by tagging anti-Ly49F Ab (IgG1) with small sized fluorescein FITC followed by anti-FITC IgG2a Abs to activate complement fixation and depletion of Ly49F+CD8 Treg.

B) CD8 Treg depletion can be also achieved using toxin-conjugated anti-Ly49F Abs. In one example, CD8 Treg depleting reagents are developed by fusing anti-Ly49F Ab with proaerolysin (PA), a potent protein toxin secreted by Aeromonas Hydrophila. A mutant version of PA (R336A) impedes PA binding to universal GPI anchors while selectivity is guided by anti-Ly49F Ab conjugates.

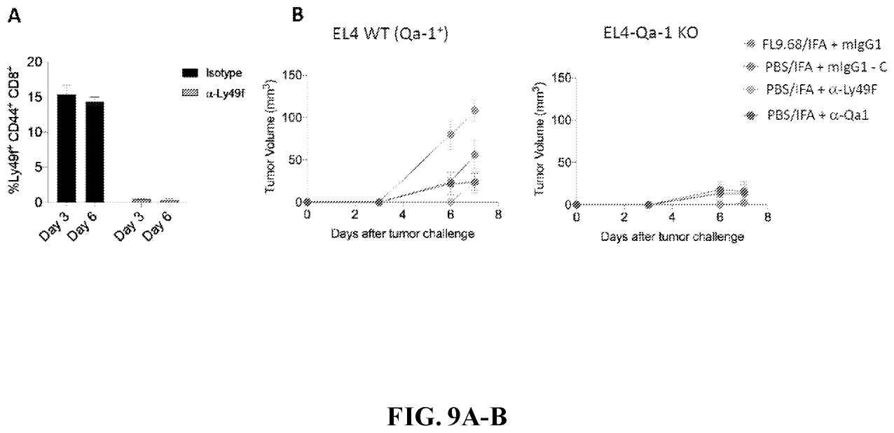

CD8 Treg deletion can be achieved using a depleting anti-Ly49F Ab (clone: HBF719). Treatment of mice inoculated with EL4 tumor cells (Qa-1⁺) with anti-Ly49F Ab resulted in significantly slow tumor growth compared to mice treated with isotype control Abs (mIgG1) FIG. 9A-B These data indicate that elimination or reduction of CD8 Treg-dependent immune suppression can enhance anti-tumor immunity.

C.) Expression/upregulation of Qa-1 is an immune evasion mechanism utilized by tumor cells. In embodiments, blockade of the Qa-1 interaction with its receptors on CD8 Treg can enhance anti-tumor immune response. This analysis reveals that treatment of mice with blocking anti-Qa-1 Abs (clone: 4C2.4A7.5H11) slows tumor growth in B6 mice (FIG. 9A).

D) In embodiments, vaccination of mice with FL9-68 SA but not IFA alone facilitates tumor growth, indicating that CD8 Treg expansion promotes tumor growth (FIG. 9B).

E.) Deletion of human CD8 Treg can be achieved by engineering anti-KIR Abs (anti-KIR3DL1 and anti-KIR2DL3) with complement-fixing isotypes (IgG2a, IgG2b or IgG3). Depletion of human CD8 Treg during immunotherapy with ICB can be a viable approach to avoid CD8 Treg-mediated inhibition of autoantibodies that promotes ICB efficacy.

### 3. Viral Infection

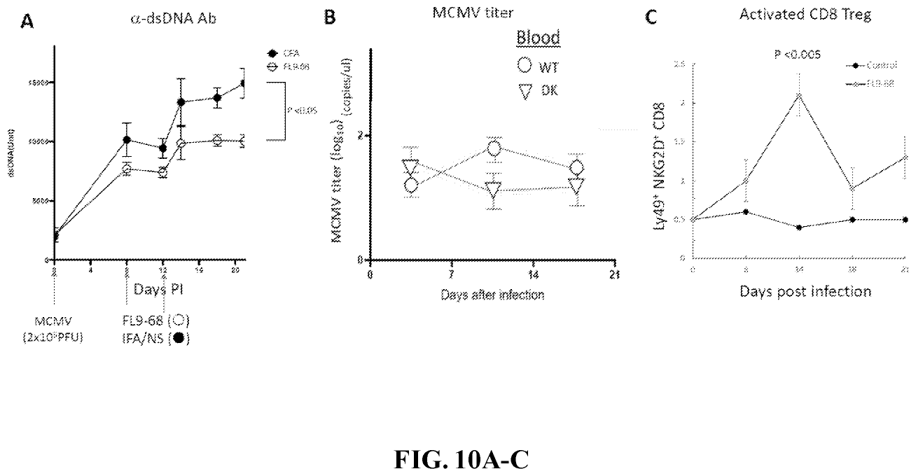

Viral infection is often accompanied by robust autoimmune responses leading to tissue damage, morbidity and, in some cases, mortality. Inhibition of self-destructive autoantibody generation by CD8 Treg mediated immune suppression represents an effective approach to dampening these infection-associated sequelae. In one embodiment, using a murine MCMV infection model, we showed that enhancement of CD8 Treg function after vaccination with peptide superagonist (FL9-68 SA at days 0, 8 and 12 after viral infection) significantly reduced production of anti-dsDNA Ab without affecting the viral clearance (FIG. 10A-C). Inhibition of anti-dsDNA Ab generation after FL9-68 SA vaccination was associated with an increase of NKG2D+Ly49⁺ CD8 T cells, consistent with expansion of activated CD8 Treg. Without being bound by theory, vaccination with SA peptides represents a viable approach to dampen autoimmune sequelae of viral infections.

### Non-Limiting Exemplary Features of the Present Disclosure:

Mobilization of CD8 Treg to regulate Ab-dependent immune response has an important advantage over general immune suppression, which may leave the host immunologically compromised. Since CD8 Treg specifically recognize cell surface antigens on Tfh cells that signal the activated status of these cells, we have developed approaches to identify superagonist peptides that efficiently mobilize CD8 Treg, reduce GC responses and suppress autoantibody generation. These include mutagenesis of cognate self-peptides, selection from libraries and testing for activation of CD8 Treg, as well as mobilization of CD8 Treg to reduce Ab-mediated autoimmune diseases.

Ly49F is uniquely expressed by CD8 Treg and blockade of this inhibitory receptor can enhance CD8 Treg activity without having impact on other cells including NK cells. Mobilization of CD8 Treg using blocking Ly49F Abs represents a highly specific approach that can be applied when suppression by CD8 Treg is most efficient including conditions of high level of autoAb generation.

CD8 Treg mainly target Tfh cells and thereby regulate Ab responses during the immune response. Efficient targeting of Qa-1-FL9 (HLA-E-FL9) by CD8 Treg after expansion with peptide agonists is applicable to ameliorate multiple immune responses characterized by pathogenic antibodies in the context of autoimmune disease, organ transplantation and infection.

Since Qa-1 (HLA-E) is upregulated on Tfh cells during robust immune responses, mobilization of CD8 Treg can be applied during the window of time when upregulation of target molecules (Qa-1-peptide, HLA-E-peptide) is maximal and CD8 Treg mobilization is most beneficial.

For the peptide vaccine, we can employ a delivery system (DSPE-PEG-peptides conjugates) that maximizes peptide delivery to lymph nodes, where peptides are taken up by DCs and presented by Qa-1, thereby increasing immunogenicity (Moynihan et al., 2018). In embodiments, DSPE (lipophilic albumin binding tail)-PEG conjugation promotes their binding to albumin (molecular chaperone) and lymphatic trafficking, resulting in robust CD8 T cell responses.

Both peptide agonists and anti-Ly49F/anti-iKIR Abs can expand CD8 Treg and these two approaches can be combined in certain embodiments to maximize CD8 Treg mobilization.

### Additional Embodiments

We have found that the FL9 peptide complexed with Qa-1 is expressed by a substantial fraction of activated Tfh cells during immune responses. Peptide mutagenesis can be applied to systemically identify synthetic peptides with superagonist activity to provoke robust expansion, activation and suppressive activity of CD8 Treg in vivo using mouse models of autoimmune disease including EAE and lupus. These peptide-based regimens can be evaluated in the context of autoimmune responses for inhibition of Ab-mediated pathogenesis and tissue damage. Without wishing to be bound by theory, since HLA-E and Qa-1 are expressed as only 1 of 2 alleles, this approach can be clinically applicable to large groups of patients and avoid the problems of MHC class Ia diversity.

Reference Cited in this Example:
Moynihan, K. D., Holden, R. L., Mehta, N. K., Wang, C., Karver, M. R., Dinter, J., Liang, S., Abraham, W., Melo, M.B., Zhang, A. Q., et al. (2018). Enhancement of Peptide Vaccine Immunogenicity by Increasing Lymphatic Drainage and Boosting Serum Stability. Cancer Immunol Res 6, 1025-1038.

### Example 3

### Super-antigen That Mobilize Regulatory Cd8 T Cells Inhibits Donor-specific Antibody And Protects Heart Allografts From Antibody-mediated Rejection

Antibody-mediated rejection (AMR) is a critical barrier to long-term allograft survival. We showed that Qa-1 (HLA-E in humans) restricted CD8⁺ T cells (CD8 Treg) play an essential role in controlling humoral immunity by killing alloreactive CD4⁺ T cells, especially follicular helper T cells (Tfh) that upregulate Qa-1 under immunologic stress conditions. We previously showed that interruption of CD8⁺ T cell receptor (TCR) binding Qa-1 unleashed Tfh proliferation and led to severe AMR in murine cardiac transplantation. In this study, we identified stresspeptides (SPs) presented on Qa-1, modified one of these peptides to engineer a super-agonist (SA), tested the efficacy of SPs in mobilizing CD8Treg. Finally, we examined if SPs subdue allo-sensitization and protect heart grafts from AMR.

Methods: Based on previous mass-spectrometry studies, we selected two SPs-FL9 and Hsp60p2l6—that associate with Qa-1 underimmunologic stress conditions. We then sorted FL9-tetramer binding CD8 Tregs, sequenced their TCR, and expressed on hybridoma. We alsogenerated a library of modified FL9 sequences and compared their antigenicity using the TCR engineered hybridoma. After selecting FL9-SA, we performed BALB/c to B6 skin transplantation with or without Hsp60p2l6 and FL9-SA, followed by heart transplantation.

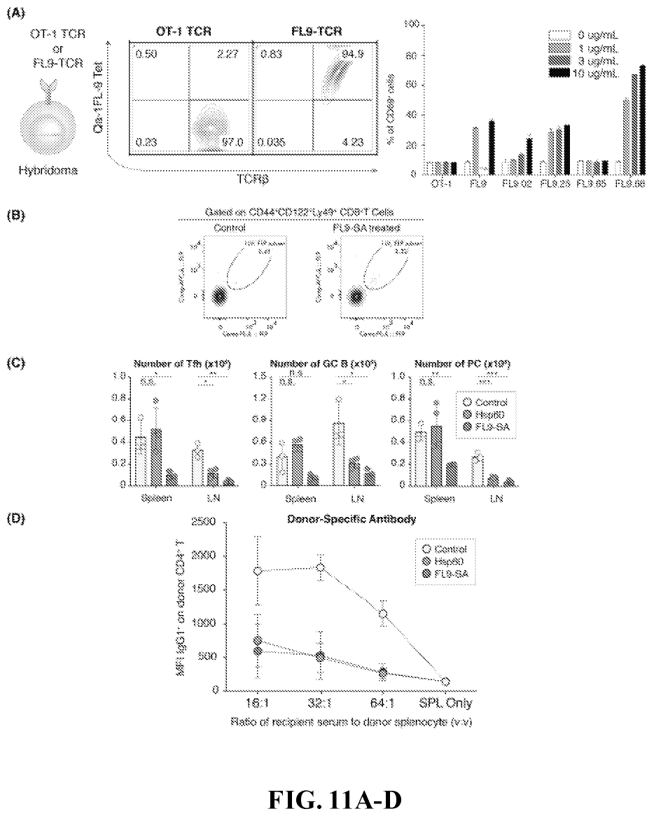

Results: We successfully generated FL9-SA using our TCR engineered hybridoma system. Immunization with SPs significantly expanded SP-Qa-1 tetramer binding CD8 Treg. Compared to the control group, hosts treated with SPs during sensitization showed a significant reduction in Tfh and mature B cells including plasma cells. FL9-SA was substantially more efficacious than Hsp60p2l6. Donor-specificantibody (DSA) was significantly decreased in SP-treated groups, resulting in protection of heart allografts (FIG. 11A-D).

Conclusion: Eliciting CD8 Treg response with Qa-1-associating SPs subdue germinal center reaction and DSA formation. Especially, the super-agonist that we generated showed superior biological efficacy in mobilizing CD8 Treg. Without begin bound by theory, exploiting the mechanism of CD8 Treg through the study of Qa-1-associating peptides offers a strategy to suppress AMR, which lacks effective therapeutic options.

### Example 4

### Mobilization of CD8 Treg: A Therapeutic Approach to Inhibiting Anti-Graft Antibody Responses in Allograft Transplantation

Antibody-mediated rejection (AMR) remains a major barrier to successful solid organ transplantation. We have developed an approach to dampen Ab-mediated injury and promote organ allograft survival based on recent advances in understanding CD8 Treg biology. The presenent disclosure of application of CD8 Treg-based therapy is relevant to at least the clinical problem of organ transplantation. Qa-1 (HLA-E in man) is a class-1b MHC molecule with a restricted polymorphism (unlike highly polymorphic class Ia MHC molecules). Murine Qa-1 is robustly expressed by activated T helper cells, especially T follicular helper (Tfh) cell, allowing targeting and lysis by CD8 Treg. This interaction regulates allo-Ab responses in a fully mismatched heart transplant model. Alloreactive Tfh cells upregulate Qa-1-self-peptide complexes, including the FL9 self-peptide expressed on a significant fraction of Tfh cells, during alloimmune responses, allowing targeting by Qal-restricted-CD8 Treg. Here we describe the use of superagonist (SA) variants of the FL9 self-peptide that have been engineered to express potent CD8 Treg stimulatory activity in association with Qa-1b. Vaccination with FL9 superagonist peptides leads to efficient mobilization of CD8 Treg and inhibition of antibody-mediated allograft rejection.

Improved strategies to dampen AMR are needed. AMR reflects a robust germinal center (GC) allo-Ab response induced by follicular T helper (Tfh) cells. Our previous studies have revealed that Qa-1 (HLA-E)-restricted CD8 Treg inhibit these Tfh-dependent GC responses (Nakagawa et al., 2018). Here, we outline an approach based on the application of superagonist self-peptides that can efficiently expand CD8 Treg, reduce GC responses and suppress antibody responses. This approach results in mobilization of CD8 Treg and reduces Ab-mediated injury to allogeneic organ transplants. Nakagawa, H., Wang, L., Cantor, H., and Kim, H.J. (2018). New Insights Into the Biology of CD8 Regulatory T Cells. Adv Immunol 140, 1-20.

CD8 Treg mainly target Tfh cells and thereby regulate Ab responses during the immune response. Efficient targeting of Qa-1-FL9 by CD8 Treg after expansion with peptide vaccine is applicable to at least organ transplantation.

CD8 Treg express an inhibitory Ly49 receptor (Ly49F, inhibitory KIR in human). The Ly49 receptors are type II C-type lectin-like membrane glycoproteins that recognize class I major histocompatibility complex-I (MHC-I) and MHC-I-like proteins on normal as well as altered cells. Without being bound by theory, since engagement of Ly49 can inhibit suppressive activity of CD8 Treg, blockade of Ly49/KIR expression may enhance CD8 Treg mobilization and increase suppressive function. In addition to peptide vaccine, Ly49F can be blocked using anti-Ly49F Abs.

### Additional Exemplary Embodiments

The FL9 peptide complexed with Qa-1 can be expressed by a substantial fraction of activated Tfh cells during immune responses. In various embodiments, peptide-based regimens will be evaluated in the context of allograft responses for inhibition of Ab-mediated injury and graft survival. Without wishing to be bound by theory, since HLA-E and Qa-1 are expressed as only 1 of 2 alleles, this approach is clinically applicable to large groups of patients and avoids the problems of MHC class Ia diversity. In embodiments, graft sensitized mice and Ag-specific heart transplant models can be used to analyze these responses.

References Cited in this Example:

Choi JY, Eskandari SK, Cai S, Sulkaj I, Assaker JP, Allos H, AlHaddad J, Muhsin SA, Alhussain E, Mansouri A, Yeung MY, Seelen MAJ, Kim HJ, Cantor H, Azzi JR. Regulatory CD8 T cells that recognize Qa-1 expressed by CD4 T-helper cells inhibit rejection of heart allografts. PNAS USA. 2020 Mar. 17; 117(11):6042-6046. doi: 10.1073/pnas.1918950117. Epub 2020 Feb 28. PMID: 32111690; PMCID: PMC7084119.

### Example 5

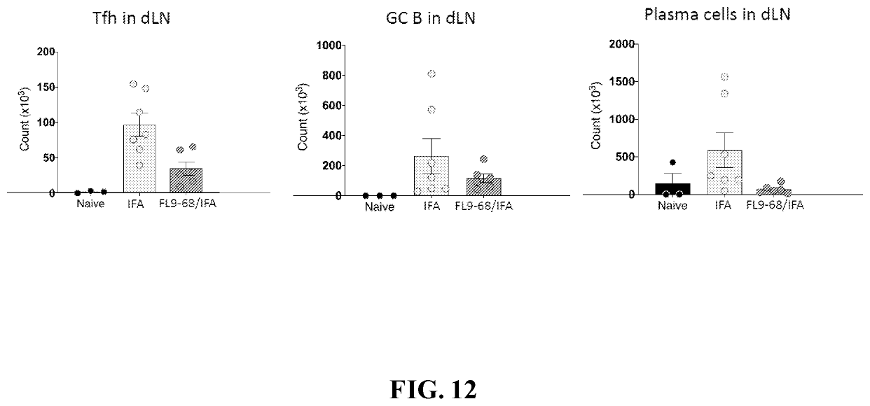

FIG. 12 The effect of peptide immunization on Tfh, GC B and plasma cell generation was tested in the heart transplantation model. Data show that there is a significant reduction of these cells in the graft recipients that were immunized with SA peptide FL9-68.

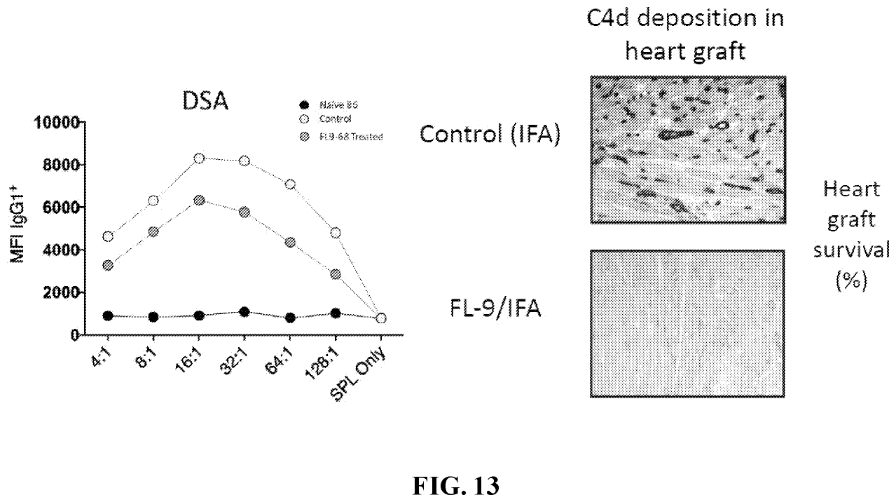

FIG. 13 FL9 SA peptide immunization leads to reduced generation of donor specific antibodies and maintenance of tissue integrity leading to heart graft survival.

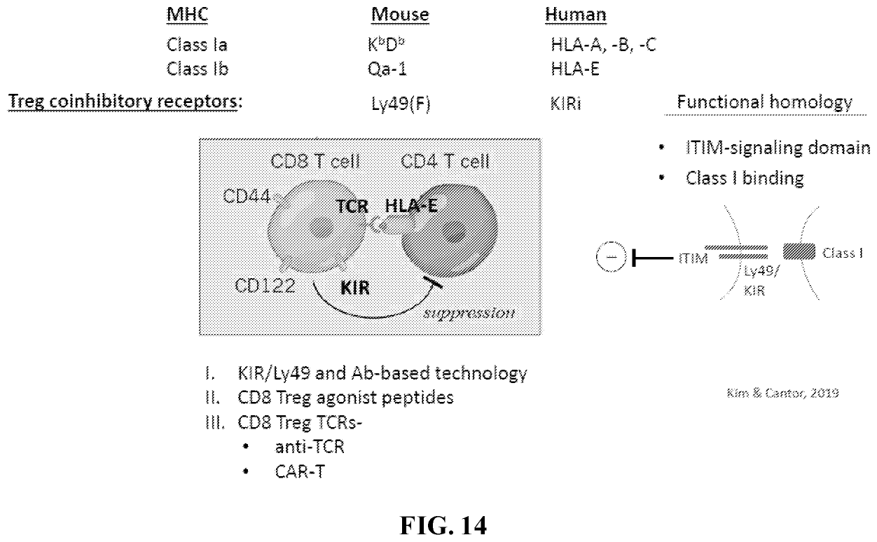

FIG. 14 CD8 Treg target HLA-E/prptide in human and Qa-1/peptide in mouse expressed on activated CD4 cells. Expression of KIR or Ly49 on CD8 Treg can inhibit their suppressive activity. CD8 Treg function can be enhanced by Ab dependent blocking of these inhibitory receptors, immunization with SA peptides or stimulating with Abs that target CD8 Treg canonical TCRs.

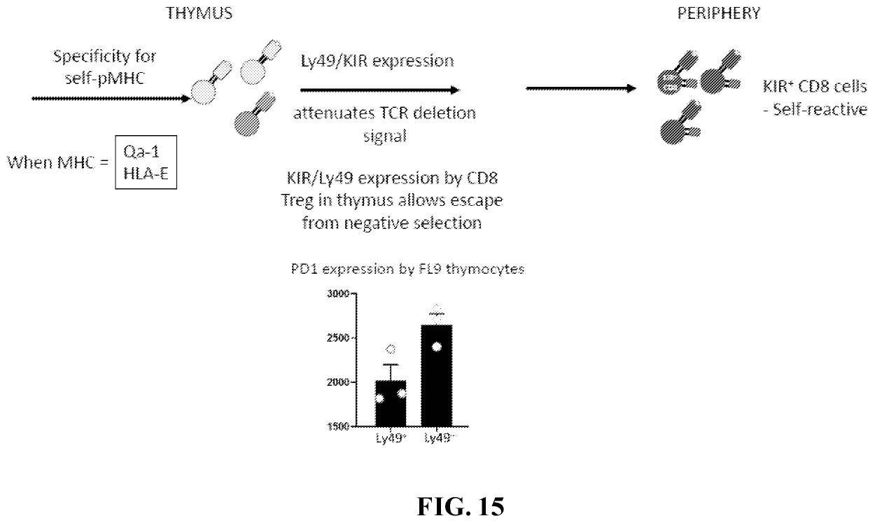

FIG. 15 CD8 Treg that express TCR with self reactivity can escape thymic negative selection by expressing inhibitory Ly49 (KIR) receptors and also PD1.

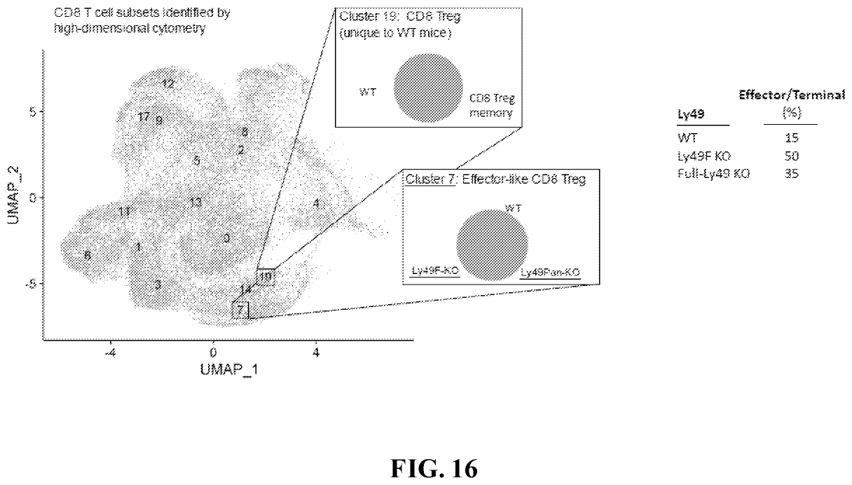

FIG. 16 Analysis of Ly49F or pan Ly49 KO CD8 Treg indicates that lack of Ly49 expression results in enhanced activation of CD8 Treg.

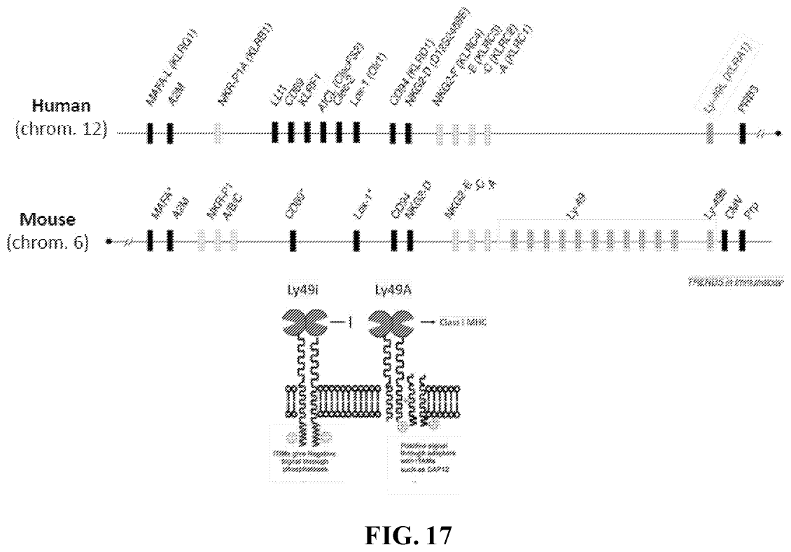

FIG. 17 Human genome is evolved to delete Ly49 locus and CD8 Treg express functional homologue KIR instead.

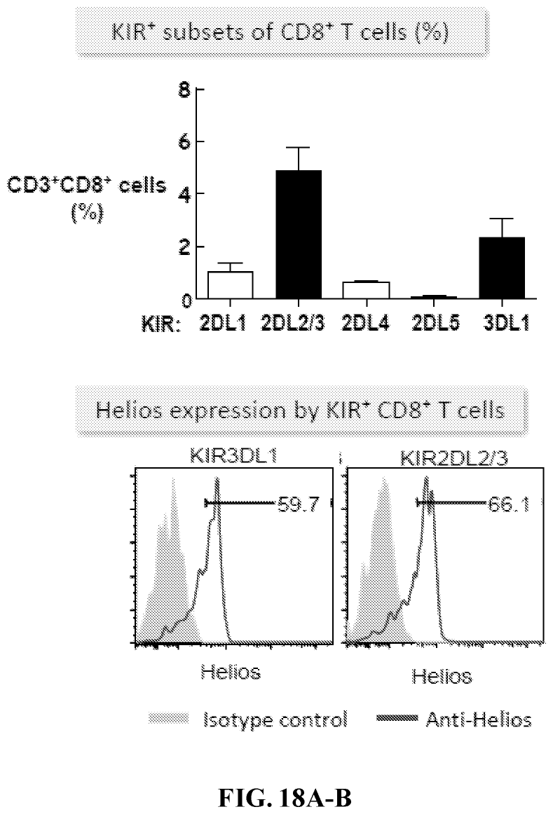

FIG. 18A-B Human CD8 Treg preferentially express KIR2DL2/3 and KIR3DL1 subtype. These KIR subtypes express Helios TF similar to murine Ly49⁺ CD8 Treg.

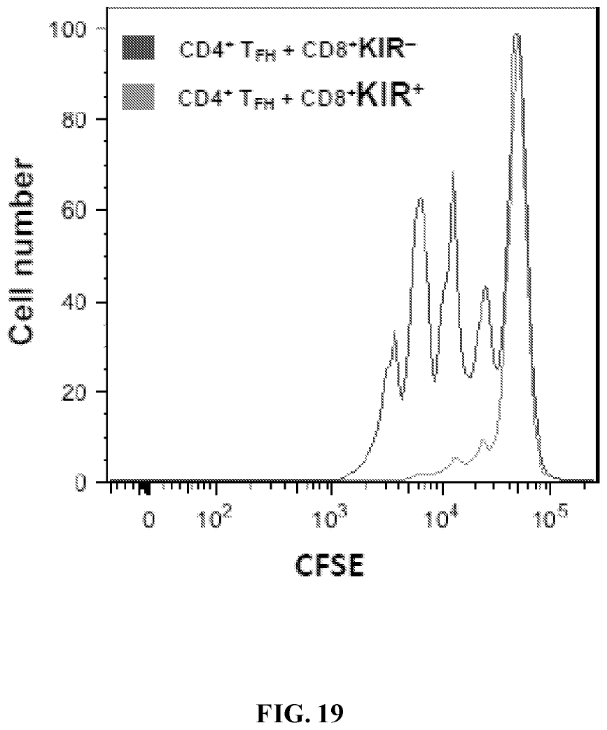

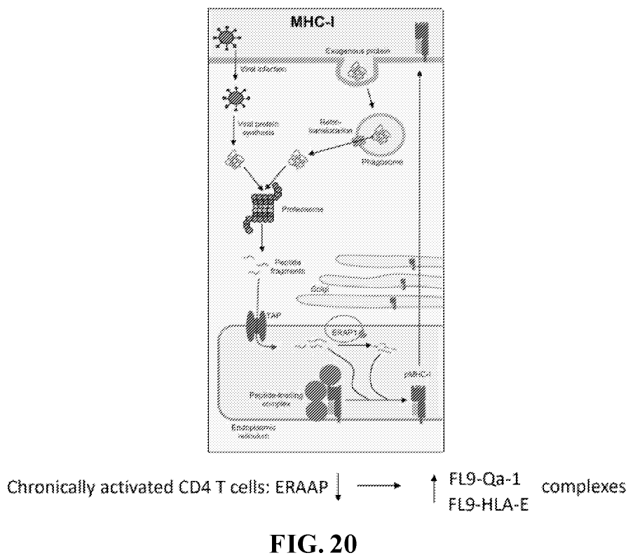

FIG. 19 KIR⁺ CD8 T cells show suppressive activity. Co-culture of isolated T_{FH} cells with CD8 T cells showed that only KIR⁺ CD8 T cells display inhibition of T_{FH} cell expansion. FIG. 20 Chronically activated CD4 cells downregulate ERAAP leading to generation of FL9 peptides that can be loaded onto Qa-1 and presented on the surface of CD4 T cells, which leads to recognition of these cells by CD8 Treg.

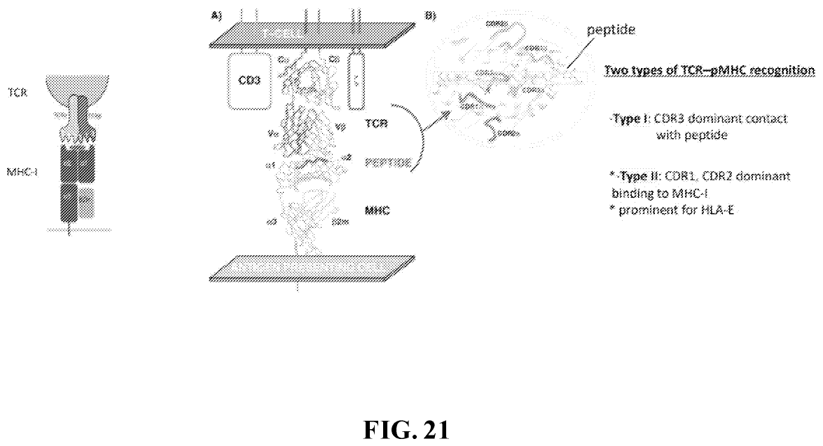

FIG. 21 TCR expressed by CD8 Treg show common V genes (CDR1 and CDR2) independent of their peptide specificity allowing TCR focus on MHC (Qa-1 or HLA-E) during development, that may allow development of these self-reactive cells by escaping thymic negative selection.

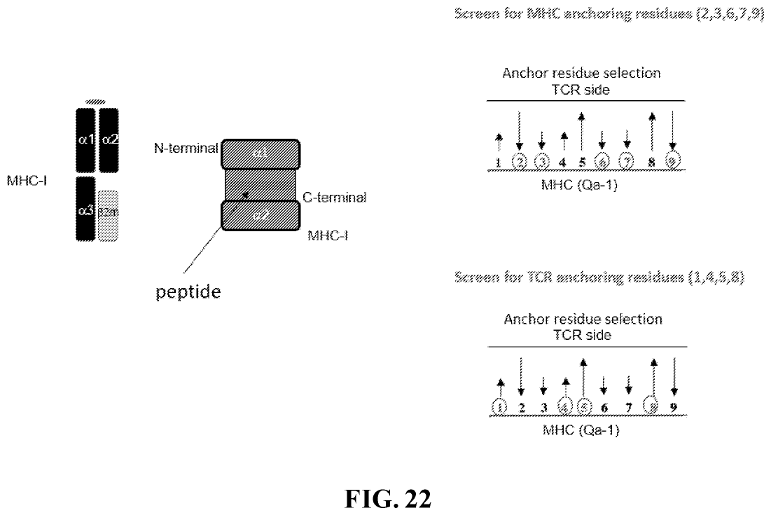

FIG. 22 Peptide superagonists for CD8 Treg can be screened in the peptide library that are composed of amino acid variants that harbor amino acid mutations in the MHC anchoring positions (2,3,6,7,9) or TCR binding positions (1,4,5,8).

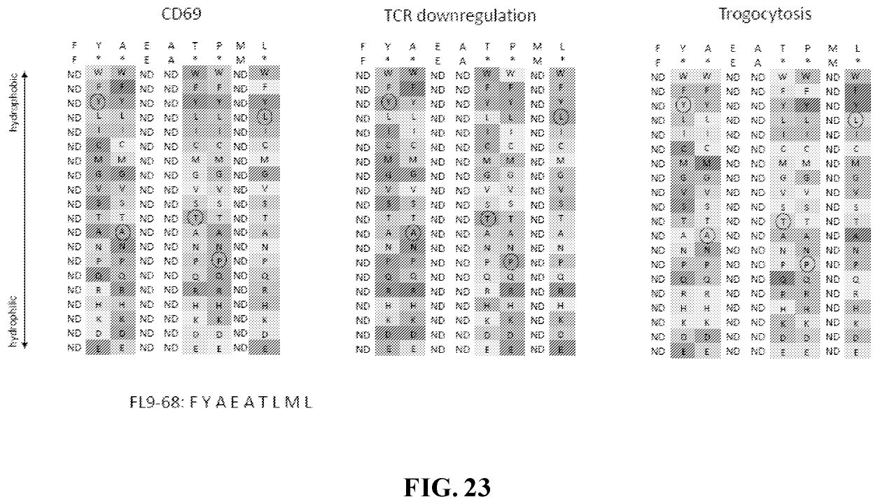

FIG. 23 FL9 peptide library that harbors variants with amino acid mutations on the MHC anchoring positions were screened for their ability to activate FL9 TCR by incubating FL9 T cells with EL4 loaded with these peptides. CD69, TCR expression and trogocytosis of TCR by APC (EL4 cells) were measured.

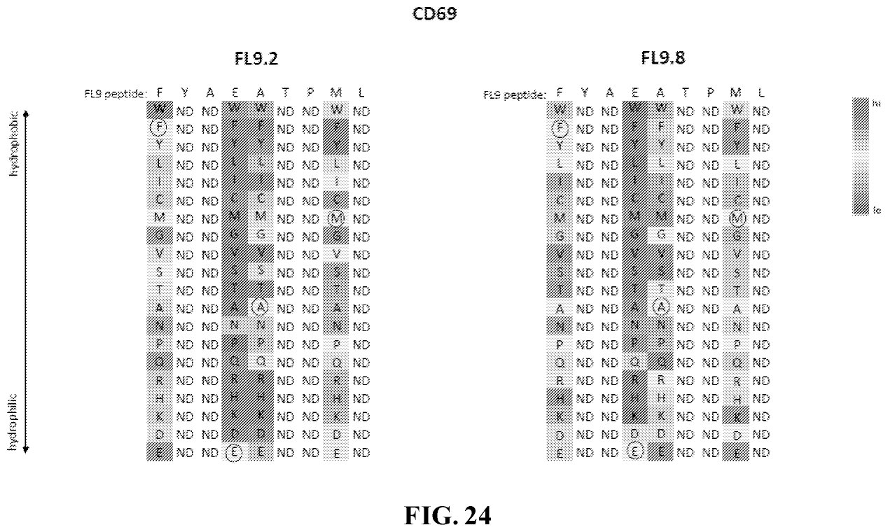

FIG. 24 FL9 Peptide library that harbor variants with amino acid mutations on the TCR binding positions were screened for their capacity to activate FL9 TCR (FL9.2 and FL9.8 TCR) by incubating FL9 T cells with EL4 loaded with these peptides. CD69 expression by FL9 T cells (both FL9.2 and FL9.8 T cells) were measured as a readout.

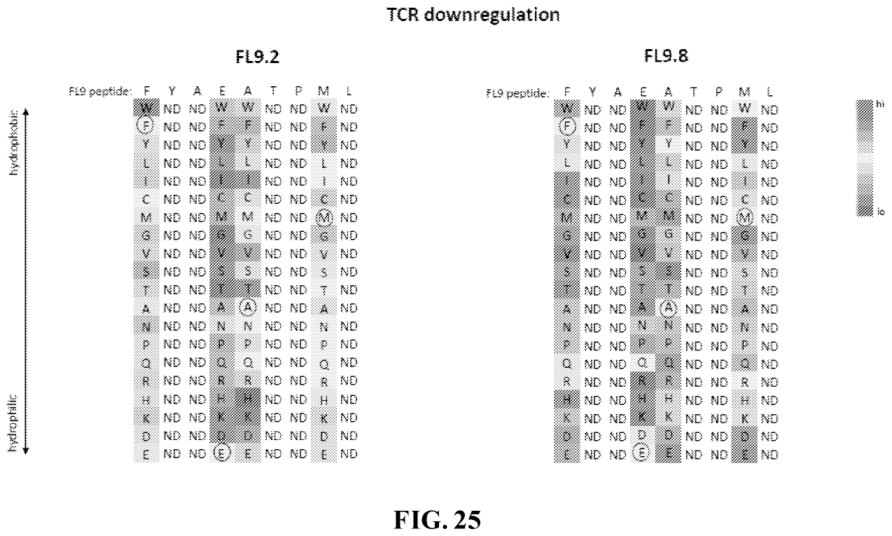

FIG. 25 FL9 Peptide library that harbors variants with aa mutations on the TCR binding positions were screened for their ability to activate FL9 TCR (FL9.2 and FL9.8 TCR) by incubating FL9 T cells with EL4 loaded with these peptides. TCR downregulation by FL9 T cells (both FL9.2 and FL9.8 T cells) was measured as a readout.

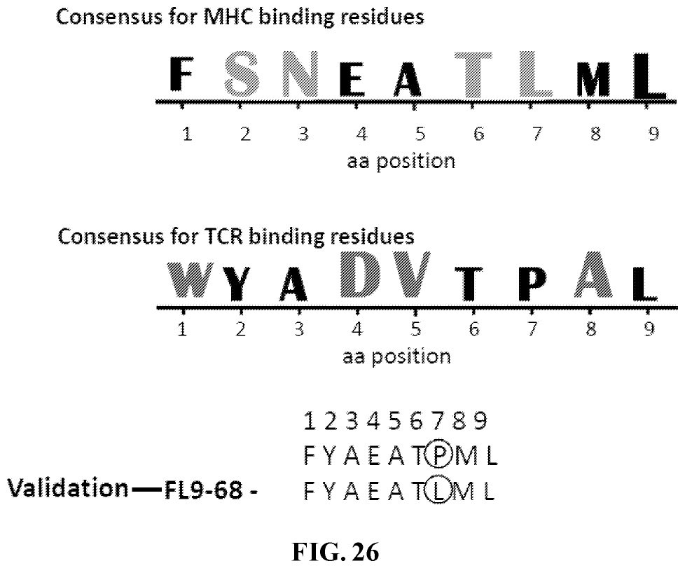

FIG. 26 Amino Acids that display the highest stimulatory ability in MHC anchoring positions or TCR binding positions are depicted as a summary. AA sequence of FL9-68 peptide that is selected for immunization for CD8 Treg activation is shown.

FIG. 27 TCR alpha chain sequences isolated from CD8 T cells that are detected by Qa-1-FL9 Tet⁺ CD8 T cells.

FIG. 28 TCR beta chain sequences isolated from CD8 T cells that are detected by Qa-1-FL9 Tet⁺ CD8 T cells.

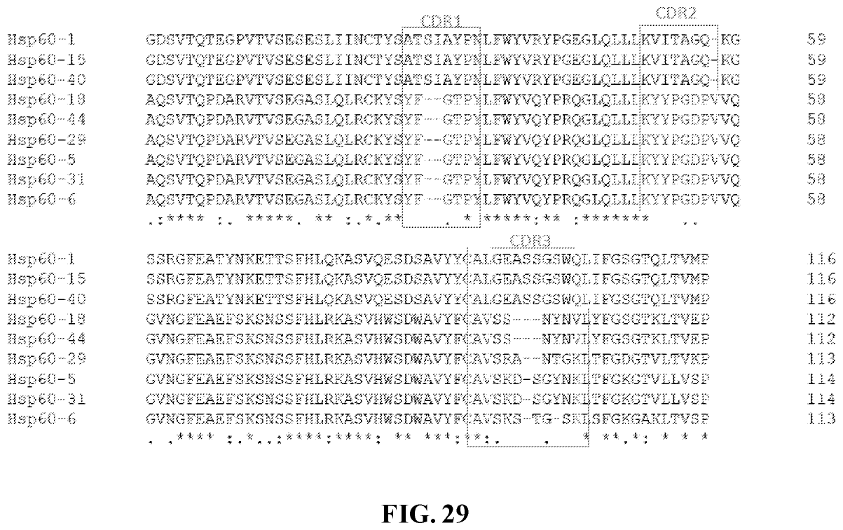

FIG. 29 TCR alpha chain sequences isolated from CD8 T cells that are detected by Qa-1-Hsp60p2l6 Tet⁺ CD8 T cells.

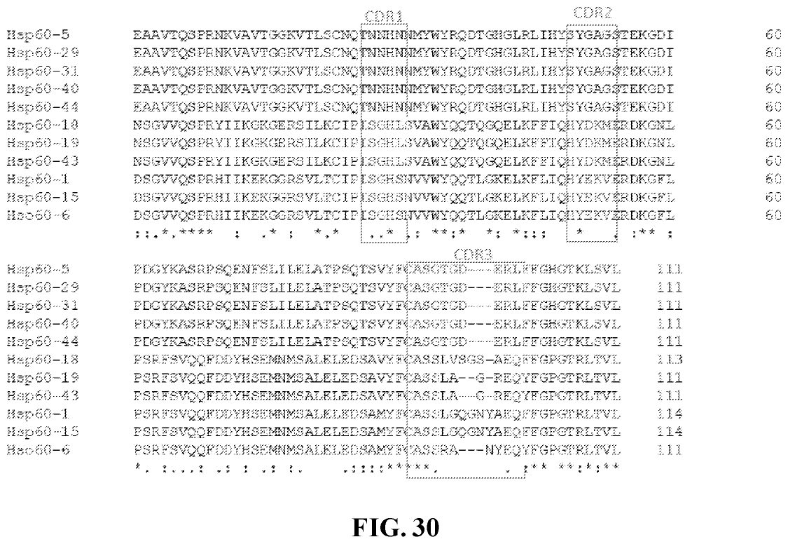

FIG. 30 TCR beta chain sequences isolated from CD8 T cells that are detected by Qa-1-Hsp60p2l6 Tet⁺ CD8 T cells.

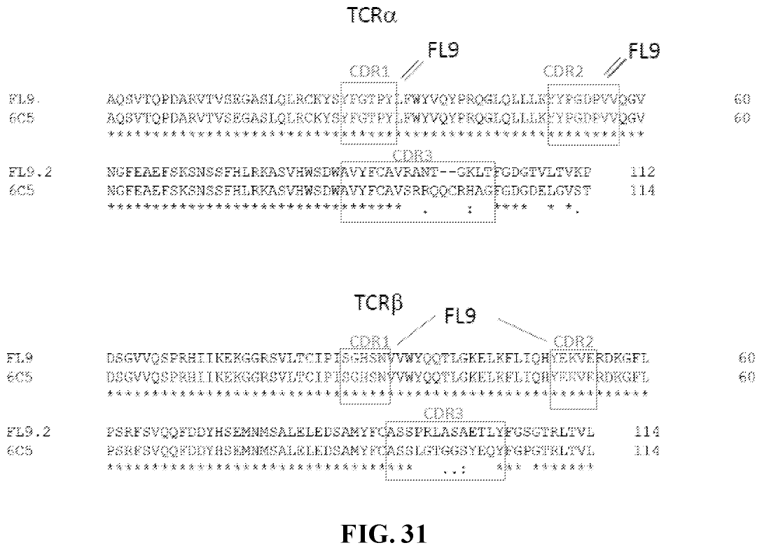

FIG. 31 Comparison of TCR alpha and beta sequences used by Qa-1-FL9 or Qa-1-pro-insulin specific CD8 T cells. Both CD8 T cell clones use Vα3.2 and Vβ5 sequences for their TCR.

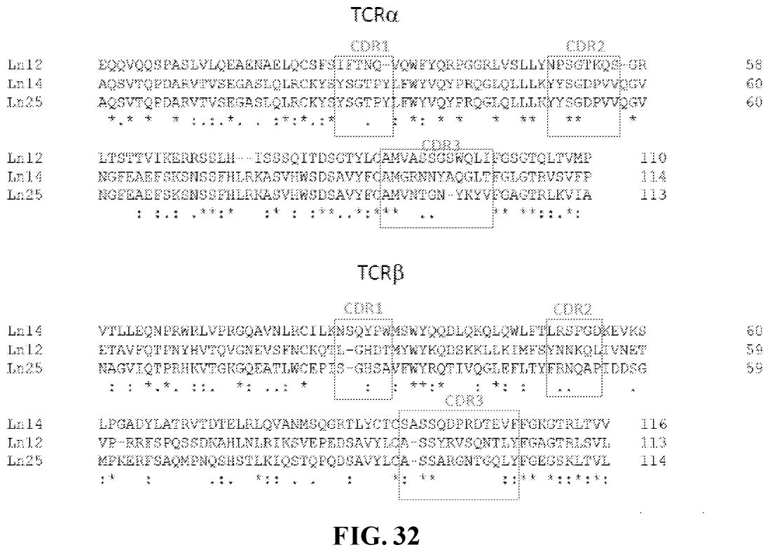

FIG. 32 TCR alpha and TCR beta sequences that are used by CD8 T cells specific for Qa-1-non-self peptide. Distinct from self-reactive Qa-1 restricted CD8 T cells, these foreign Ag specific Qa-1 restricted CD8 T cells do not use Vα3.2 and Vβ5 for their TCR.

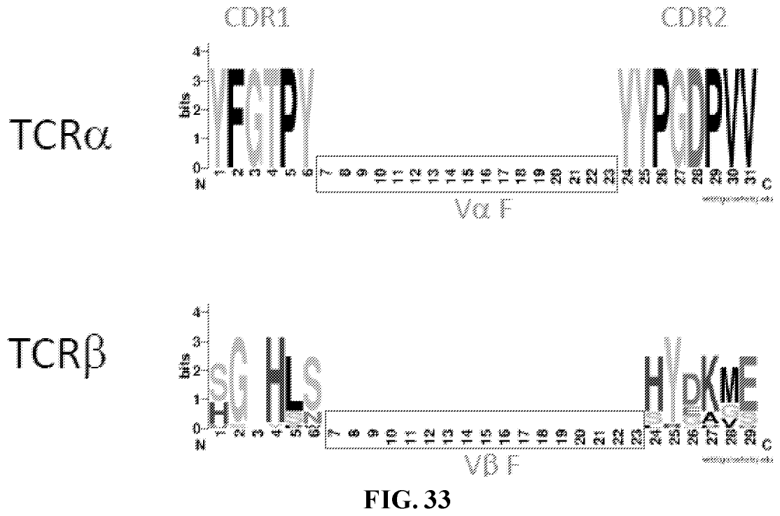

FIG. 33 Conserved CDR1 and CDR2 sequences for TCR alpha and TCR beta chains used by CD8 Treg.

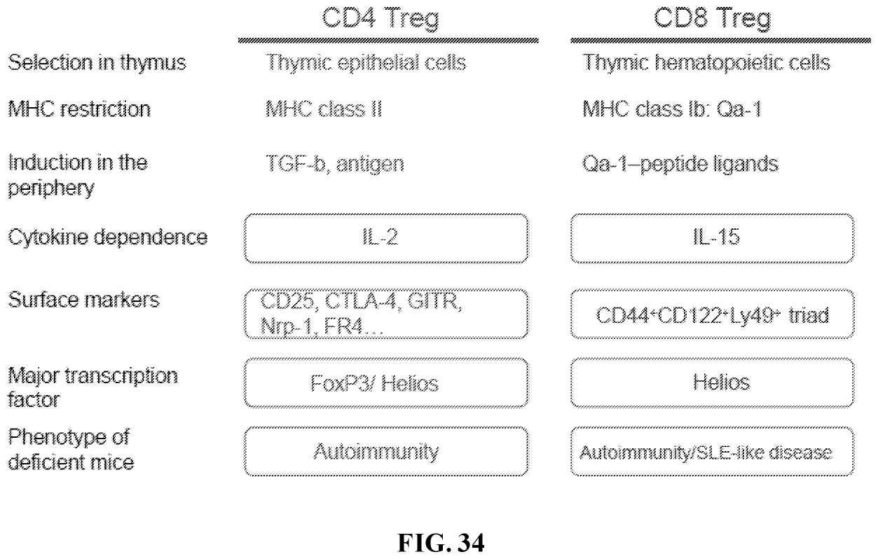

FIG. 34 Both CD4 and CD8 lineages contain regulatory T cells that are critical for the maintenance of immune homeostasis. CD8 Treg can be identified by CD44, CD122 and Ly49 surface markers and express Helios transcription factor. CD8 Treg are dependent on IL-15 for their survival and activity. Continuous recognition of Qa-1-self-peptiode may be critical for their maintenance in the peripheral T cell pool.

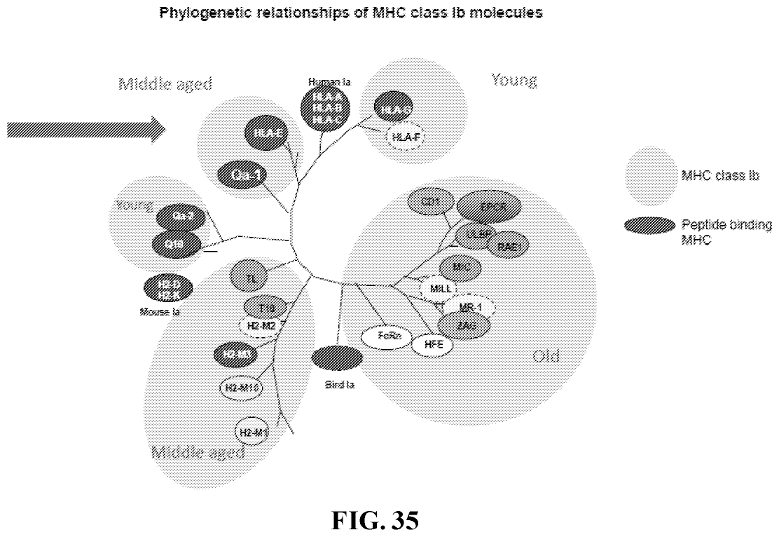

FIG. 35 HLA-E and Qa-1 have evolved to present peptides among MHC class Ib molecules similar to conventional MHC class I molecules. The capacity of Ag presentation allows these MHC molecules to signal their cellular status (activation, stress, transformation) that can be recognized by CD8 Treg.

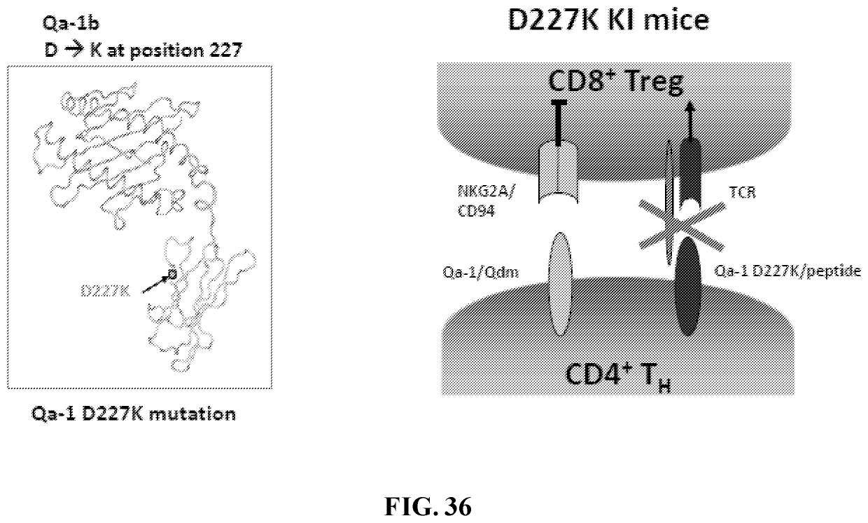

FIG. 36 B6.Qa-1.D227K KI mice were generated by mutating the AA position 227 on Qa-1 from D→K resulting in disruption of CD8 coreceptor binding. Qa-1.D227K mutants do not have CD8 Treg mediated immune regulation and represent an important genetic model to study physiological function of CD8 Treg.

FIG. 37 Using a Balb/C→B6 heart transplantation model, we demonstrated accelerated rejection of allografts in the absence of CD8 Treg mediated immune suppression (Qa-1.D227K mice). Heart graft tissue in D227K recipients showed immune cell infiltration and C4d deposition, indicating enhanced immune response to heart allograft.

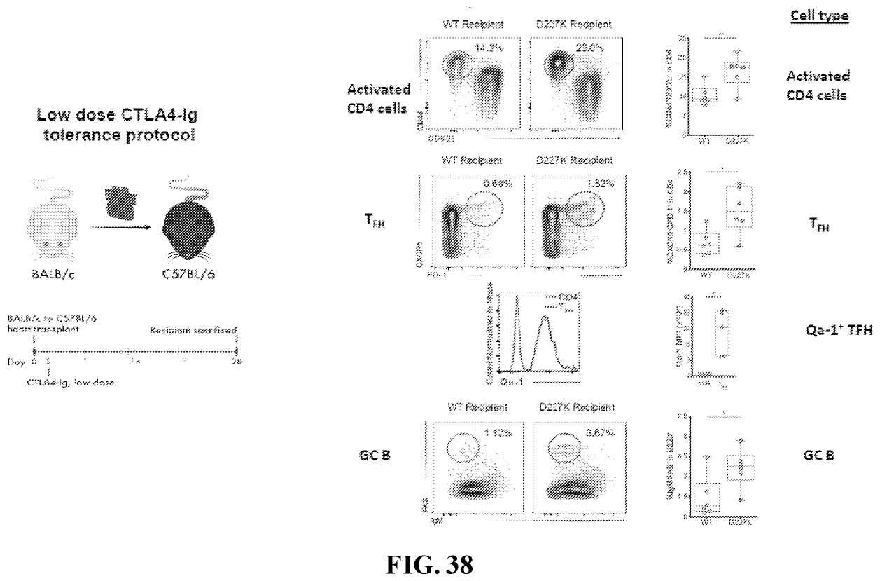

FIG. 38 D227K mice show enhanced Ab responses to heart allograft as evidenced by increased Tfh and GC B cell formation. Tfh cells express high levels of Qa-1 during immune responses to allograft rendering them susceptible to CD8 Treg mediated suppression.

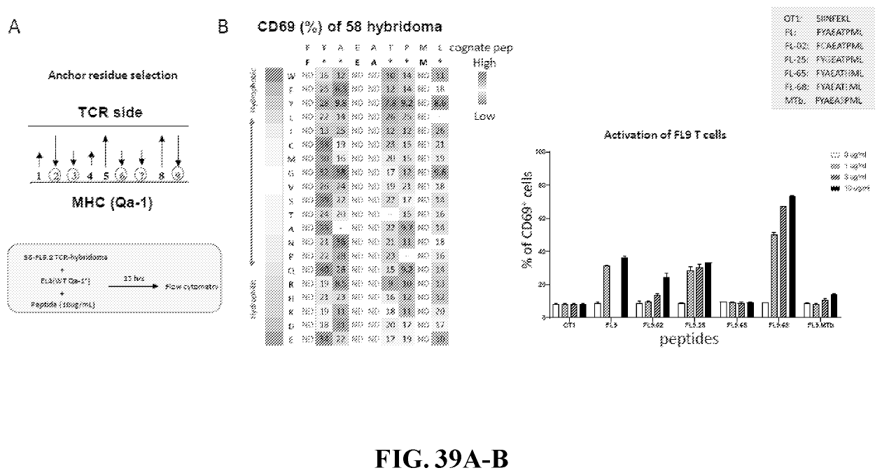

FIG. 39A-B FL9 Peptide library that harbor variants with aa mutations on the MHC anchoring positions were screened for their ability to activate FL9 TCR by incubating FL9 T cells with EL4 loaded with these peptides. CD69 expression by FL9 T cells was measured. Selected peptides were tested for their FL9 T cell stimulatory ability with titrated dose of peptides. FL9-68 peptide showed superior ability to stimulate FL9 T cells.

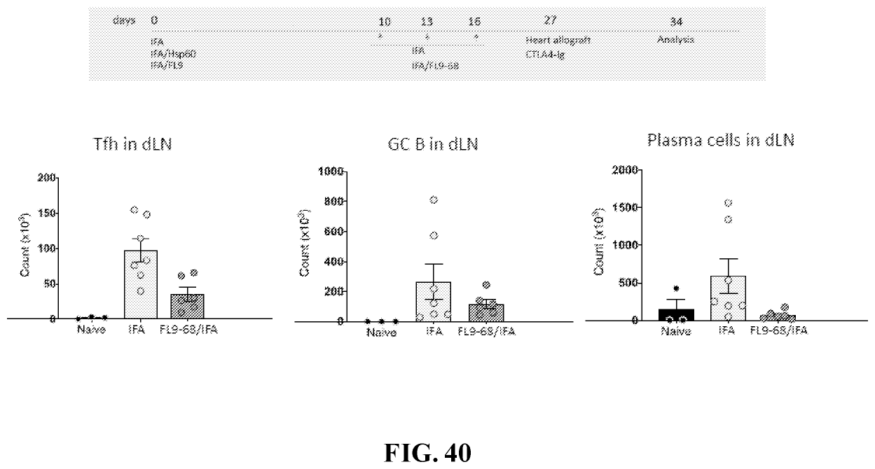

FIG. 40 Immunization of heart graft recipients with FL9-68 peptides resulted in decreased Tfh, GC B and plasma cell formation, indicating suppression of anti-allo immune responses.

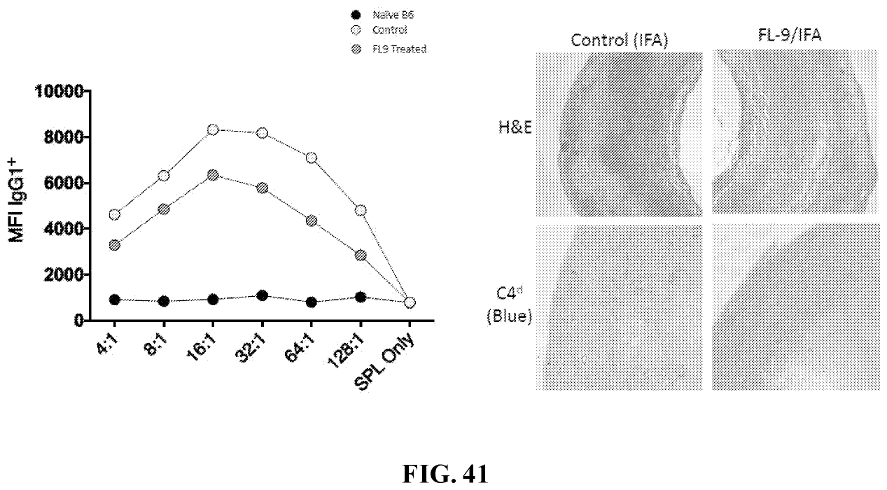

FIG. 41 Immunization of heart graft recipients with FL9-68 peptides resulted in decrease of donor specific Ab (DSA) generation and graft tissue damage, indicating CD8 Treg mediated suppression of anti-allo immune response.

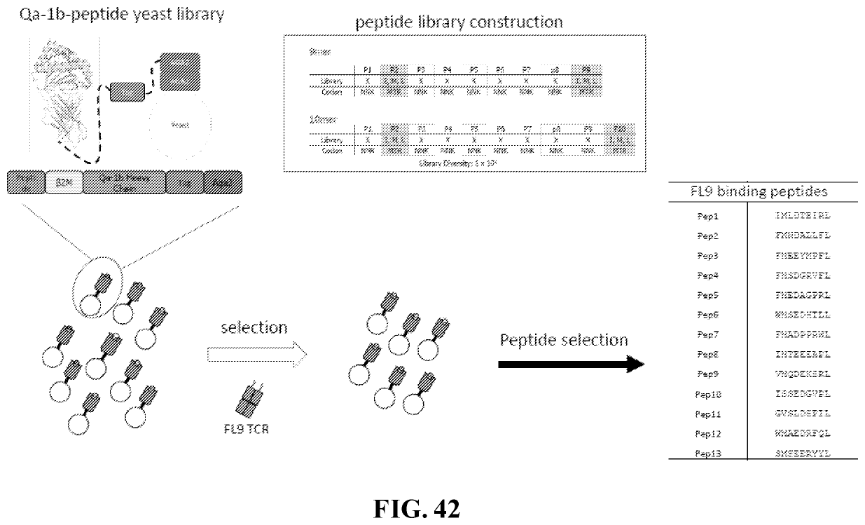

FIG. 42 Qa-1 peptide yeast library composed of 1×10⁸ 9 mer and 10 mers of ramdom peptides was constructed to screen a large pool of peptides that allowed identification of multiple peptide superagonists.

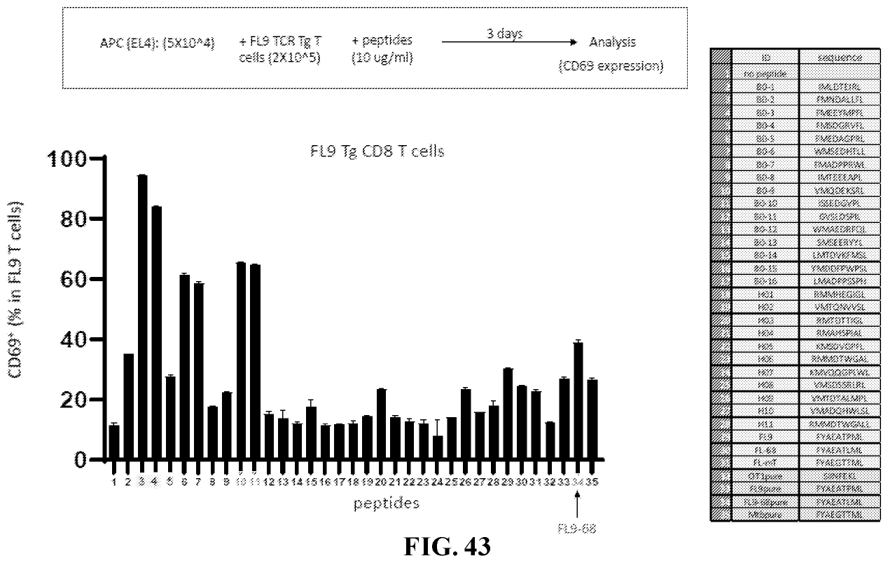

FIG. 43 Screen of the Qa-1-peptide yeast library led to identification of peptides that display superior capacity to stimulate FL9 T cells. These peptides can be tested their in vivo activity to expand CD8 Treg upon immunization.

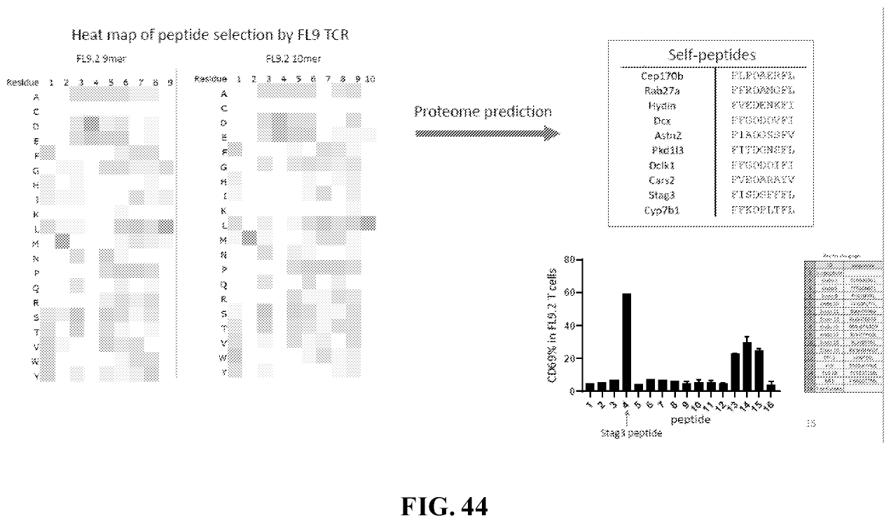

FIG. 44 Surrogate peptides selected from Qa-1-peptide yeast library were subjected to the endogenous peptide search, resulting in identification of candidate self-peptides. Among these candidate peptides, a peptide derived from Stag3 protein showed a capacity to stimulate FL9 T cells after in vivo loading onto EL4 cells.

### Example 6

### Working Title: Definition of the MHC Class Ib Restricted Subset of CD8⁺ Regulatory T Cells According to TCR Gene Expression and Target Cell Recognition

### Abstract

Although most CD8⁺ T-cells are equipped to kill cells infected by microbial invaders, a subset of these cells may suppress immune responses (Nakagawa et al., 2018; Saligrama et al., 2019)(Nakagawa et al., 2018; Saligrama et al., 2019). Murine and human CD8 regulatory activity are invested in a small (<5%) subset of CD8 T cells that express a characteristic triad of surface receptors: CD44, CD122 and Ly49/KIR (triad+). Analysis of autoimmune disorders has revealed that these CD8 T regulatory cells (CD8 Treg) inhibit disease through targeting of MHC class Ia or class Ib expressed by CD4+ T-helper cells. However, whether CD8 Treg that target class Ia or class Ib represent distinct subsets is not known.

Analysis of a panel of more than 30 independent TCRs expressed by Qa-1-restricted CD8 T cells specific for two structurally-distinct self-peptides revealed predominant usage of TRAV9N3 and TRBV12-1/2 genes encoding TCR Vα3.2/Vβ5.1. Development and function of Ly49F+Vα3.2/Vβ5.1+ was almost completely abrogated in Qa-1-deficient mice, indicating that the Qa-1-restricted subset of CD8 Treg is confined to CD8 cells expressing the Vα3.2/Vβ5.1 TCR. Genetic Ab-based targeting of Qal-restricted CD8 Treg after immunization with OVA resulted in a selective increase in numbers of high affinity tetramer+OVA CD4 T cells and antibody responses.

The study indicates that the TCRs expressed by virtually all Qa-1 (MHC-E) restricted CD8 Treg are encosed by a highly conserved set of Vu+V3 genes that detect and eliminate target CD4 T cells without generalized immune suppression. Insight into the TCR-based specificity of CD8 Treg has led to new therapeutic approaches using synthetic peptide agonists to mobilize CD8 Treg to inhibit pathogenic or autoimmune Ab responses.

### Introduction

The immune system has evolved complex mechanisms that allow efficient destruction of microgial pathogens while sparing the host's own tissues. Maintenance of this delicate balance depends, in part, on regulatory T cells. Although most CD8⁺ T-cells are equipped to kill cells infected by microbial invaders, there is increasing evidence that a subset of CD8⁺ T-cells is genetically programmed to suppress immune responses (Nakagawa et al., 2018; Saligrama et al., 2019). Murine and human CD8 regulatory activity are invested in a small (<5%) subset of CD8 T cells that expresses a characteristic triad of surface receptors: CD44, CD122 and Ly49/KIR (Kim et al., 2011; Saligrama et al., 2019) and mediate perform-dependent killing of chronically-activated and autoreactive CD4 cells (Saligrama et al., 2019; Vivier and Anfossi, 2004). Analysis of autoimmune disorders has revealed that CD8 T regulatory cells (CD8 Treg) inhibit disease through recognition of MHC class Ia (Saligrama et al., 2019) or class Ib (Nakagawa et al., 2018) on target CD4⁺ T-helper cells. However, whether CD8 Treg that recognize self peptides associated with class Ia or class Ib represent distinct or overlapping subsets is not known. Here we define the development of class Ib-restricted CD8 Treg and distinguish them from class Ia-restricted CD8 Treg according to TCR expression and thymus-dependent development.

Although recognition of MHC-E (human HLA-E or murine Qa-1)-peptide complexes expressed by target CD4 cells is required for regulatory activity, the identity of TCRs that recognize class Ib (Qa-1) target ligands and associated self-peptides is not known. Processing and cell surface expression of Qa-1 pMHC complexes by activated T cells depends on trimming by several enzymes, including an endoplasmic reticulum aminopeptidase associated with antigen processing (ERAAP), which digests larger peptides into 9/10mers that can efficiently bind to Qa-1. Shastri and colleagues showed that diminished or defective ERAAP activity associated with chronic activation of CD4 T cells prevents destruction of a Qa-1-associated self-peptide termed FL9 and promotion of FL9-specific memory CD8 T cells (Lazaro et al., 2009; Nagarajan et al., 2012).

In addition to the FL9 self-peptide, chronically-activated CD4 T cells also express self-peptides derived from the Hsp60 protein associated with Qa-1 that allow targeting by CD8 Treg (Leavenworth et al, 2013). To define the TCRs used for recognition of these pQa-1 complexes, we cloned and analyzed two large sets of TCR expressed by CD8 Treg that bound to either Qa-1-FL9 or Qa-1-Hsp60 tetramers. Analysis of a panel of more than 30 independent TCRs expressed by Qa-1-restricted CD8 T cells specific for these two structurally-distinct self-peptides revealed enrichment of the same TRAV and TRBV genes encoding Vα3.2 and Vβ5.1, respectively.

Analysis of mice that express TCR transgenes and non-transgenic mice revealed that mutation or deletion of Qa-1 had a profound impact on the maintenance and activation of the Vα3.2⁺/Vβ5.1⁺ fraction of triad+ CD8 T cells but had no detectable effect on the Vα3.2⁻/Vβ5.1⁻ triad+ fraction. Additional analysis indicated that development of the Qa-1-restricted CD8 Treg subset required TCR recognition of pQa-1 by conserved TRAV and TRBV genes for both thymic-dependent differentiation and maintenance in peripheral lymphoid tissues. The MHC-E focus of conserved TCR CDR1/2 sequences may allow these self-peptide-specific T cells to escape negative selection in the thymus and mediate efficient recognition and elimination of Qa-1+ CD4+ Th cells within peripheral tissues.

We used systematic peptide mutagenesis at the MHC-contact residues of the FL9 self-peptide to identify synthetic superagonist peptides that would promote efficient mobilization of CD8 Treg in vivo. Indeed, immunization with these FL9 superagonist peptides promoted robust expansion of CD8 Treg and efficient inhibition of Tfh-driven Ab responses to conventional antigens and in a preclinical model of solid organ transplantation. Moreover, selective deletion of Qa-1-restricted Treg by an anti-TCR Ab that recognized the conserved TRAV (Vα3.2) after immunization with a conventional Ag (OVA), resulted in a marked increase in Qa-1hi CD4 T cells that had a relatively high avidity for cognate Ag, without affecting normal CD4 T cell activation.

The study indicates that the TCRs expressed by virtually all Qa-1 (MHC-E) restricted CD8 Treg are distinguished by a conserved set of Vα⁺Vβ⁺ genes that include germline CDR1/CDR2 sequences that may mediate efficient interactions with MHC-E-self-peptide complexes expressed by target cells. Sensitive detection of changes in MHC-E expression by pathogenic target CD4 cells can mediate elimination of pathogenic CD4 T cells without generalized immune suppression. The TCR-based specificity of CD8 Treg indicates new therapeutic approaches to dampen pathogenic or undesired Ab responses.

### Results

### Identification of TCRs Specific for Qa-1-FL9 Self Peptide

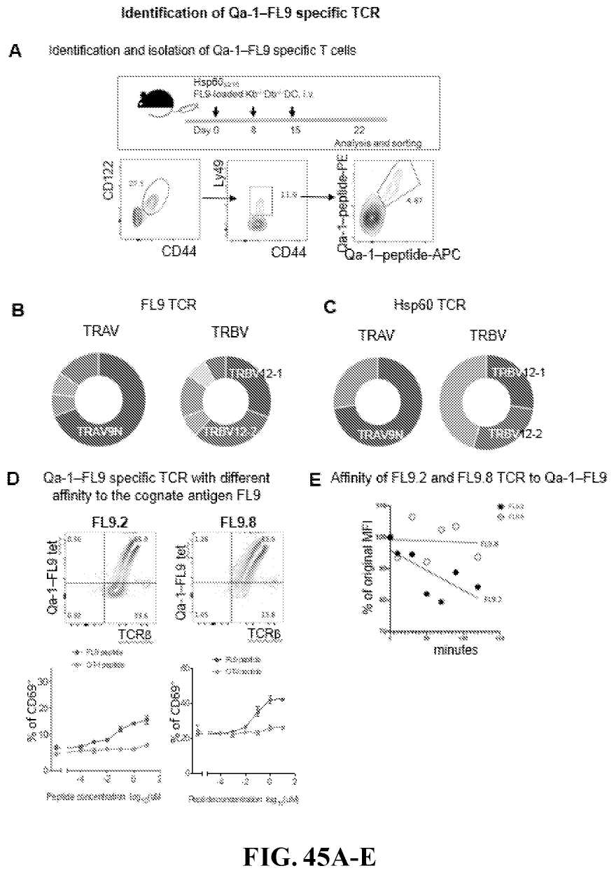

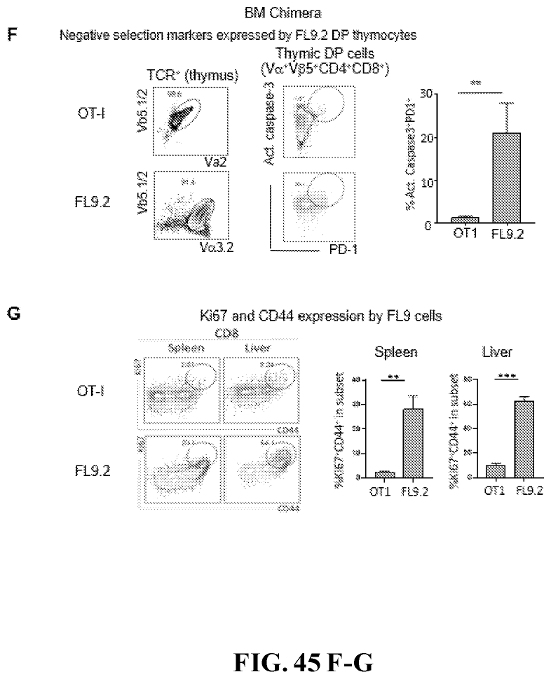

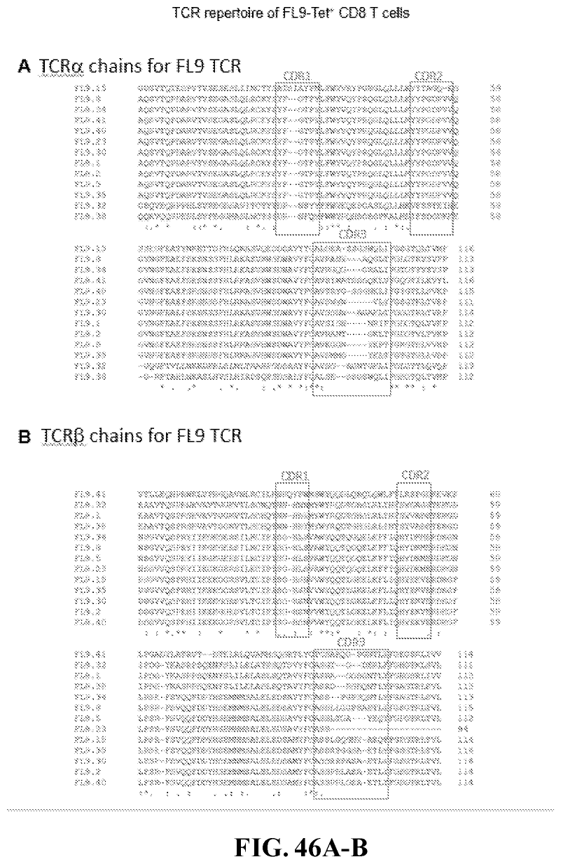

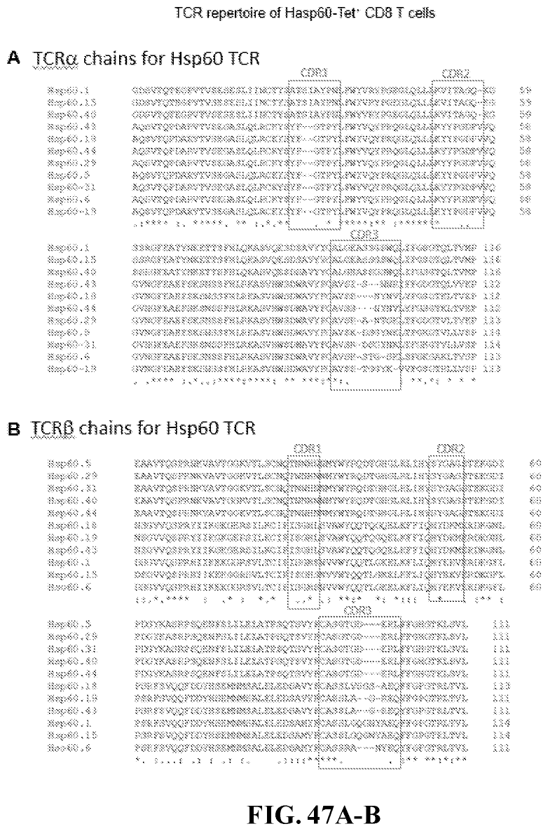

Insight into the specialized function of both class Ia- and class Ib-restricted CD8 Treg has relied mainly on isolation of both subsets of CD8 Treg using a triad of shared surface markers—CD44, CD122 and Ly49. Here we distinguish class Ib (Qa-1-restricted) CD8 Treg from class Ia-restricted Treg according to expression of TCR specific for two structurally-unrelated self-peptides—FL9 and Hsp60, which associate with Qa-1 to allow targeting of pQa-1-bearing CD4 cells (Leavenworth et al., 2013; Nagarajan et al., 2012; Nakagawa et al., 2018). We used Qa-1-FL9 and Qa-1-Hsp60 peptide tetramers to detect, sort and analyze TCR expression by tetramer-positive (tet+) cells according to single-cell TCR sequencing (FIG. 45A). Analysis of paired TCRs from 13 independent Qa-1-FL9 tetramer-binding cells revealed that 10/13 TCR specific for Qa-1-FL9 tetramers expressed the TRAV9N3 gene (Vα3.2) and 9/13 expressed TRBV 12-1/2 (Vβ5.1,2) (FIG. 45B, FIG. 46A-B). Analysis of 11 independent Qa-1-Hsp60-specific CD8 T cells revealed that 8/11 also expressed TRAV9N3/Vα3.2, and 6/11 expressed the TRBV 12-1/2/Vβ5.1,2 (FIG. 45C, FIG. 47A-B). Both TCR sets expressed nearly identical CDR1 and CDR2 sequences which interact with MHC contacts, but distinct peptide-specific CDR3 regions. This conserved TCR repertoire used to recognize two structurally-distinct peptides indicates that Qa-1 restriction relies on a highly constrained interaction between Qa-1 and the CDR1/CDR2 MHC contact regions of the TCR and with peptide diversity dependent on the CDR3 region.

### TCR-Dependent Acquisition of CD8 Treg Phenotype by Qa-1-FL9 Specific T Cells

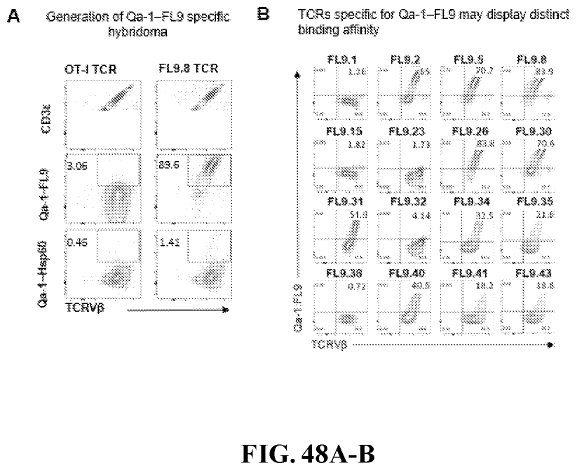

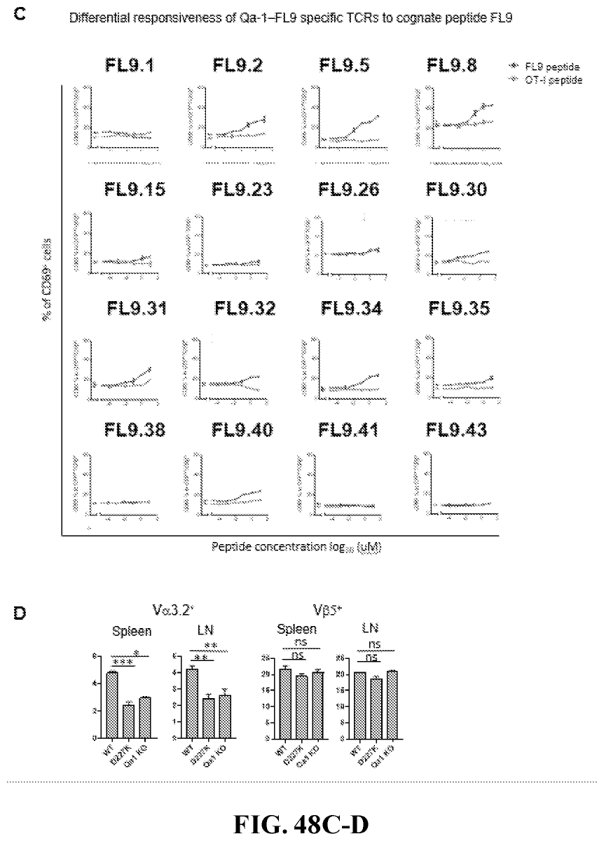

To gain further insight into the contribution of TCR usage to the differentiation and function of self-reactive CD8 Treg, we cloned each of the 16 TCR pairs specific for Qa-1-FL9 into retroviral vectors and expressed them in 58C (α′β′) hybridoma cells. Expression of each TCR in 58C cells was accompanied by specific binding to Qa-1-FL9 but not Qa-1-Hsp60 tetramers (FIG. 48A-B), presumably reflecting the peptide-specific CDR3 sequences noted above (FIG. 46A-B). The binding avidity of each Qa-1-FL9-specific TCR was then determined according to a dose-response analysis of the concentration of FL9 peptides required for CD69 upregulation by each transduced hybridoma (FIG. 48C). A Qa-1-FL9-specific TCR with an intermediate (FL9.2) avidity, and high avifity (FL9.8) for Qa-1-FL9 (FIG. 45D) were defined further in an antigen dissociation assay, which confirmed the higher affinity of the FL9.8 TCR, as judged by increased retention of Qa-1-FL9 tetramer compared with the FL9.2 TCR (FIG. 45E).

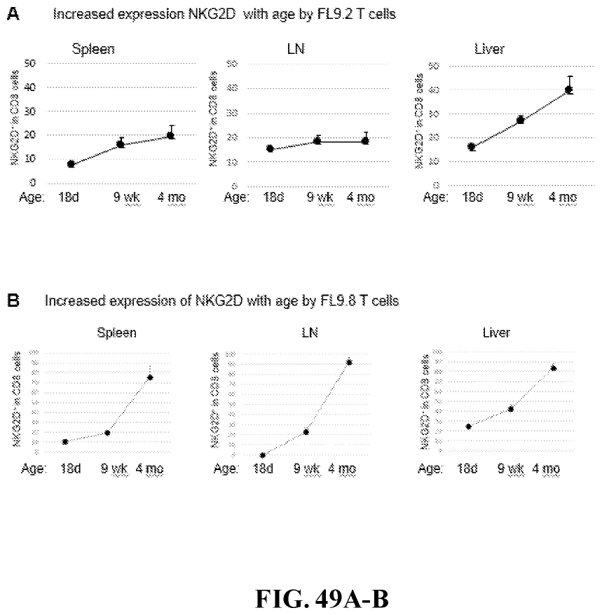

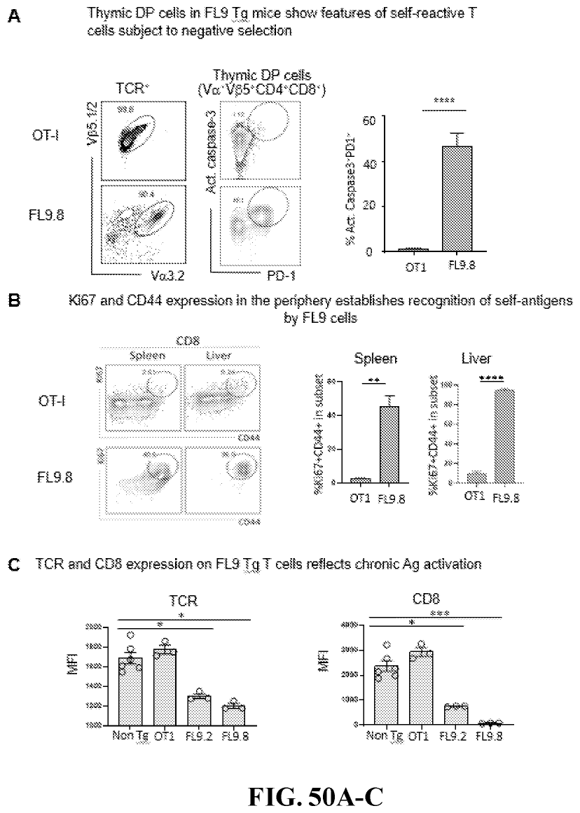

We then generated BM chimeras after reconstitution of lethally-irradiated B6 hosts with BM transduced with OT-I, FL9.2 or FL9.8 TCRs to study the contribution of these TCRs to the selection and development of CD8 Treg. Expression of the FL9.2 and 9.8 self peptide-specific TCRs used to generate TCR Tg mice using methods employed previously to generate OT-I TCR Tg mice depended on insertion of (pES.42.1c and pKS913.CD18.31) vectors (Hogquist et al., 1994). The percent of Tg TCR⁺ T cells in peripheral tissues was ˜90% of the three chimeras that had been reconstituted with each TCR transgene (FIG. 45F, 49A). Analysis of thymocytes revealed that approximately 20% of FL9.2 and 40% of FL9.8 thymocytes expressed markers of negative selection that included caspase 3 and PD1, in contrast to OT-I thymocytes which did not express these negative selection markers (FIG. 45F, 50A). Analysis of peripheral T cells revealed that the two FL9 TCR transgenes but not OT-I displayed increased expression of CD44 and Ki67 (FIG. 45G, 50B) and reduced levels of TCR and CD8, i.e., a CD8 T cell phenotype associated with chronic activation by self antigens (Schonrich et al., 1991; Xiao et al., 2007) (FIG. 50C). Chronic exposure of CD8 T cells to self-antigen may also upregulate expression ofNKG2D receptors (Dhanji et al., 2004; Zloza et al., 2011), which has been correlated with autoreactivity and potential immunoregulatory function (Dai et al., 2009). FL9.8 T cells displayed an age-dependent upregulation of NKG2D expression (>80% at 4 mo), while FL9.2 T cells displayed a more modest increase (20-40%) (FIG. 49A, 49B).

### Qa-1 Dependent Differentiation and Maintenance of FL9 T Cells

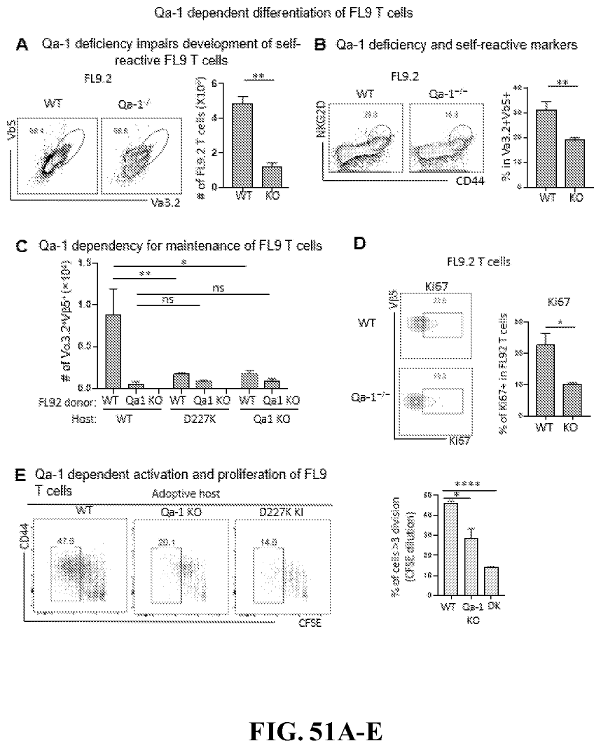

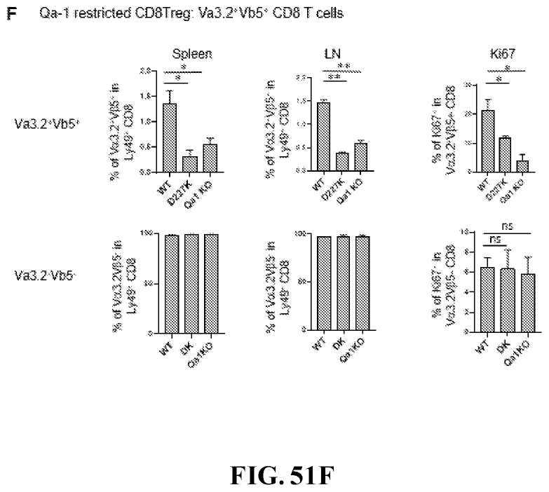

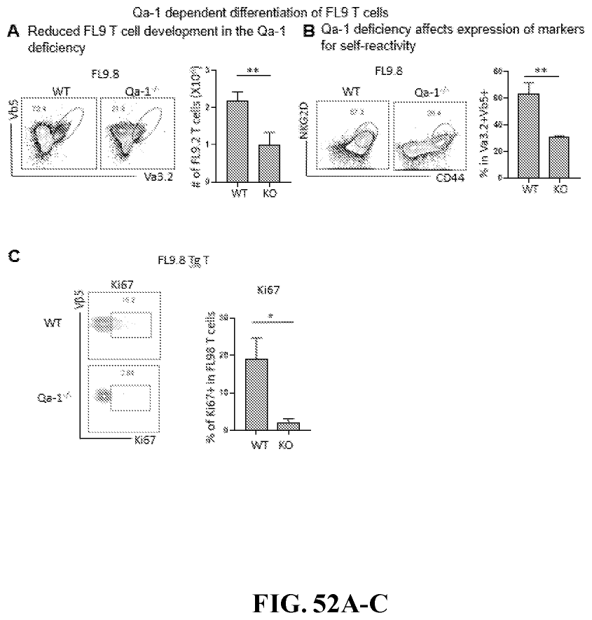

We then analyzed the contribution of Qa-1 to acquisition of the CD8 Treg phenotype in Qa-1 WT and Qa-1 KO FL9.2 and FL9.8 TCR Tg mice. Deletion of the Qa-1 restriction element resulted in an approximately 80% reduction in the numbers of FL9.2 CD8 T cells (FIG. 51A), as well as the higher affinity FL9.8 TCR⁺ T cells (FIG. 52A). CD44 and NKG2D expression (FIG. 51B, 52B) as well as the Ki67 proliferation marker were markedly impaired in both FL9.2 (FIG. 51D) and FL9.8 T cells (FIG. 52C) peripheral lymphoid tissues.

Although numbers of TCR Tg FL9.2 T cells were reduced by 70-80% in mice that expressed defective or deleted Qa-1, a significant fraction remained. We asked whether these residual TCR Tg CD8 cells in the spleen and lymph node of Qa-1-deficient mice were functionally impaired. Transfer of residual FL9.2 T cells from Qa-1^{-/-} mice into irradiated adoptive Qa-1 WT hosts revealed that very few (˜10%) survived compared with the robust survival of FL9 T cells from Qa-1 WT donors (FIG. 51C). We then asked whether recognition of Qa-1 in peripheral tissues was essential for continued survival of mature Qa-1-restricted FL9 T cells that had initially differentiated in a Qa-1-sufficient (Qa-1 WT) environment. We found that FL9 CD8 T cells from Qa-1 WT donors transferred into Qa-1 KO or D227K KI hosts that express a Qa-1 D227K point mutation that impairs the interaction between Qa-1 and the CD8 co-receptor displayed poor survival that was similar to that of CD8 TCR tg cells transferred from Qa-1-deficient donors (FIGS. 51C, 51D, 51E). These data indicate that an initial intrathytmic development of Qa-1 restricted CD8 Treg requires expression of Qa-1 and b) survival of Qa-1-restricted T cells in peripheral tissues also requires continued expression of the Qa-1 restriction element.

### Vα3.2⁺ CD8 T Cells Represent Qα-1 Restricted CD8 T Cells

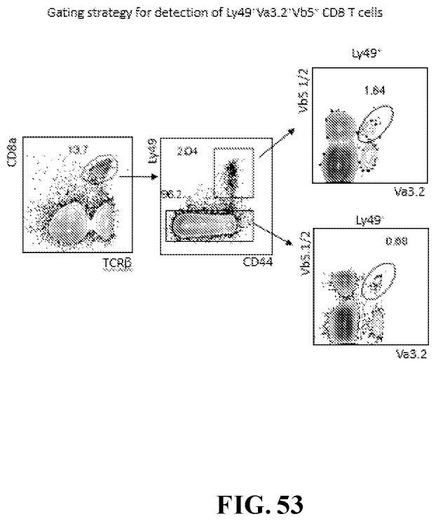

Analysis of TCR expression by Ly49⁺ CD8 T cells that bind to the FL9 or Hsp60 peptide assiocated with Qa-1 revealed a highly restricted TCR Vu and VP gene expression (FIGS. 45A-B; 46A-B1, 47A-B2). Analyses of CD8 Treg in non-transgenic mice indicate that CD8 Treg expressing the CD44⁺ CD122⁺ Ly49⁺ triad includes both MHC class Ia- and class Ib-restricted CD8 Treg (Kim et al., 2011; Saligrama et al., 2019). We therefore asked whether selective expression of the Vα3.2 and Vβ5 TCR pair noted above for Qa-1-restricted CD8 Treg might distinguish the MHC class Ib-restricted Treg subset of CD8 Treg in non-transgenic mice. We found that the Vα3.2⁺/Vβ5.1,2⁺ subset of triad+CD8 T cells were reduced by 60-80% in mice carrying a Qa-1 deletion or Qa-1 D227K point mutation (FIG. 53). In contrast, the numbers or percent of Vα3.2/Vβ5.1,2⁻ triad+(Ly49⁺ CD122⁺ CD44⁺) CD8 cells were not significantly affected by Qa-1 deletion or mutation (FIG. 51F). Finally, the residual ˜20-30% of Vα3.2⁺/Vβ5.1,2⁺ CD8 cells in Qa-1 KO mice displayed a 75% reduction in Ki67 marked proliferation. Taken together, these findings indicate that Qa-1-restricted CD8 Treg are distinguished by expression of a conserved set of TCRs as well as the Ly49⁺CD44⁺CD122⁺ marker triad and are equipped to recognize structurally-unrelated self-peptides.

### Detection and Elimination of Antigen-Specific CD4 Cells by Qa-1-Restricted CD8 Treg

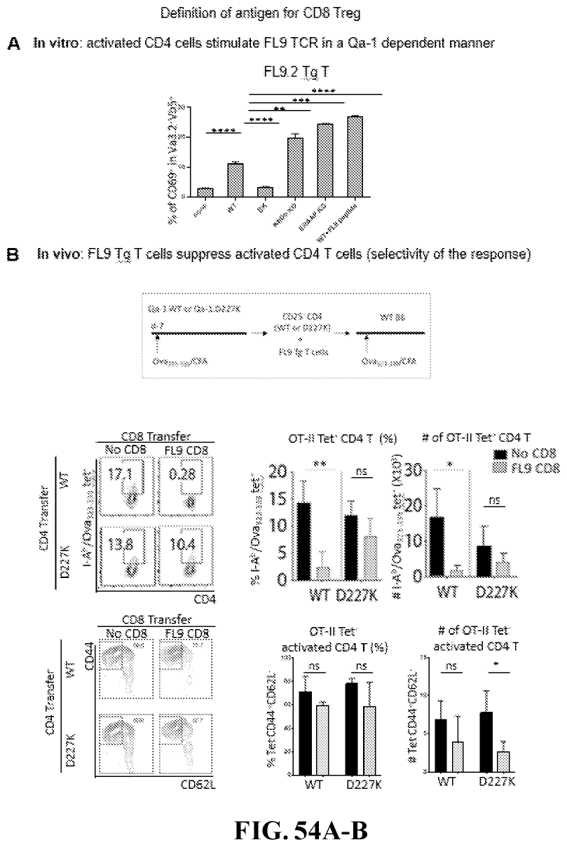

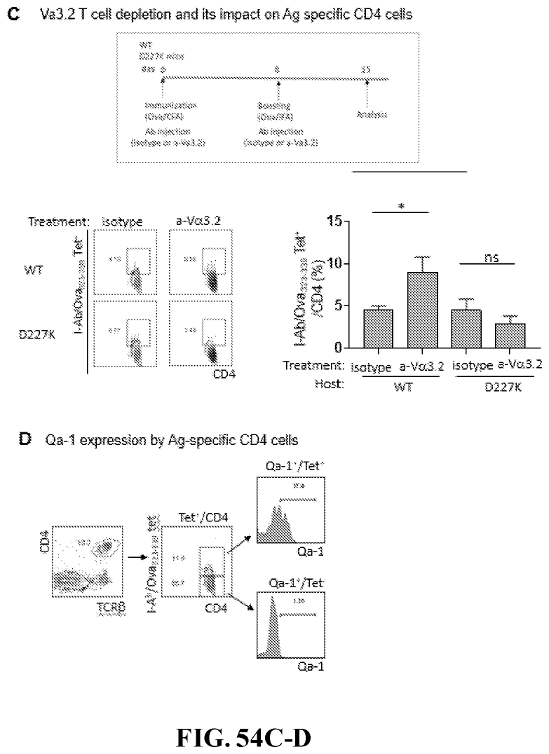

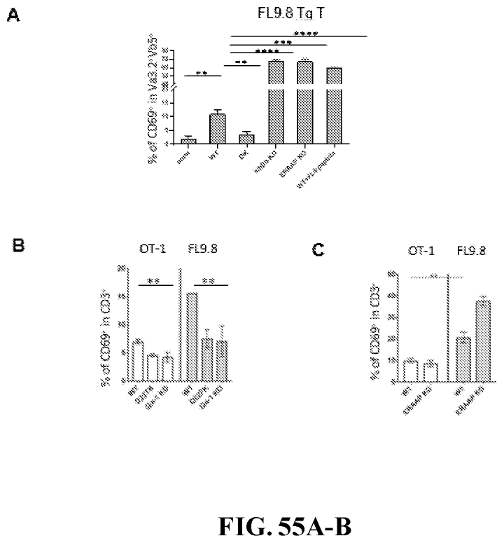

Although targeting of CD4 cells by CD8 Treg may reflect TCR-dependent recognition of pQa-1 complexes expressed by activated CD4 cells (Nakagawa et al., 2018), the nature of the target complexes is not well understood. Here we asked whether expression FL9-Qa-1 complexes represented a major functional target on Ag-specific CD4 T cells. Analysis in vitro indicated that FL9 TCR Tg T cells are efficiently stimulated by activated CD4 T cells from B6 (Qa-1 WT) mice but not B6.Qa-1 KO or B6.Qa-1-D227K KI mice (FIG. 54A). Moreover, Kb^{-/-}Db^{-/-} CD4 cells provoked increased responses by FL9 TCR⁺ CD8 T cells, reflecting the absence of a dominant Qdm default peptide derived from MHC class Ia that binds to Qa-1 (FIGS. 54A, 55A, 55B) and (2) activated ERAAP-deficient CD4 cells strongly stimulated FL9 TCR Tg T cells, consistent with increased Qa-1-FL9 expression by cells lacking the ERAAP protease, which normally destroys this peptide (Nagarajan et al., 2012) (FIGS. 54A, 55C). Collectively, these findings indicate that activated CD4 cells express an ERAAP-sensitive Qa-1-FL9 ligand that is recognized by Qa-1-FL9-specific CD8 Treg.

CD4 cells with high affinity TCR for antigen may express high levels of Qa-1 (Fazilleau et al., 2009; Nakagawa et al., 2018). To examine whether CD8 Treg may selectively target activated CD4 T cells with high affinity for immunizing or environmental antigens, we characterized CD4 cells generated after immunization according to expression of Qa-1-FL9 and sensitivity to inhibition by CD8 Treg in vivo. We transferred CD4 cells from OT-II-peptide-immunized WT B6 or B6-D227K mice into B6 hosts with or without FL9 TCR Tg CD8 cells, followed by immunization with OT-II/CFA. Analysis of OT-II tetramer* CD4 cells, which represent CD4 cells with the highest avidity for immunizing Ag, revealed that co-transfer of FL9 TCR Tg CD8 T cells inhibited more than 90% of Ova-specific (tetramer+) CD4 cells (FIG. 54B, upper panel), while virtually all activated CD4 cells that were tetramer-negative were spared (FIG. 54B, lower panel). Suppression of Ag-specific CD4 cells depended on Qa-1 targeting, since FL9 CD8 T cells suppressed the response of B6 (WT) CD4 T cells but not B6-D227K CD4 T cells (FIG. 54B, upper panel). These data indicate that a) the Qa-1-FL9 peptide complex is expressed on a substantial proportion of CD4 cells after activation by Ag, and b) CD4 cells expressing highly avid TCR (tetramer+) for cognate Ag strongly express Qa-1 (Qa-lhi) and are specifically suppressed by Qa-1-restricted CD8 Treg.

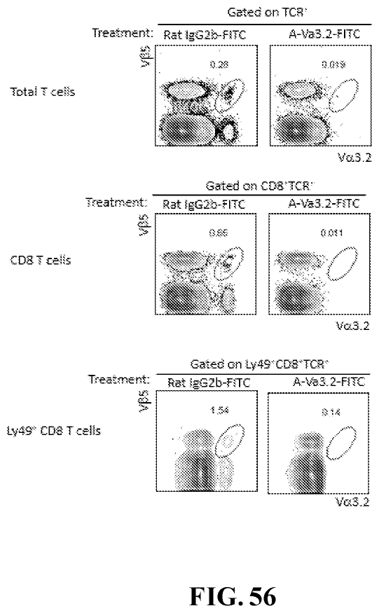

Based on the observation that Qa-1-restricted CD8 Treg express the Vα3.2/Vβ5 pair (FIG. 51F), we asked whether Ab-mediated depletion of Vα3.2⁺T cells after immunization to OVA might enhance the Ag-specific CD4 T cell responses. Virtually complete depletion of Vα3.2⁺ T cells could be achieved based on the blood analysis on day 8 after anti-Vα3.2 Ab administration (FIG. 56). Moreover, B6.WT but not B6.Qa-1 D227K mice that had been immunized with OVA/CFA and boosted with OVA/IFA developed an increased frequency of Ova-specific CD4 cells (I-Ab/Ova₃₂₃₋₃₃₉ Tet⁺) after depletion of Vα3.2⁺ T cells (FIG. 54C). Markedly increased levels of Qa-1 expressed by I-Ab/Ova₃₂₃₋₃₃₉ Tet⁺ CD4 cells, that were significantly higher than the total Tfh subset, indicates that CD8 Treg may selectively suppress high affinity Ag-specific CD4 cells (FIG. 54D). These data indicate that Qa-1-restricted CD8 cells express the Vα3.2/Vβ5 pair which can be targeted to selectively delete Qa-1-restricted CD8 Treg.

### Definition of FL9-Superagonistpeptides

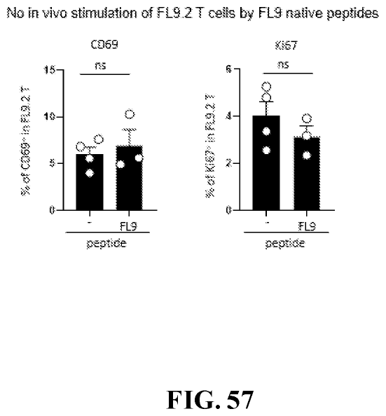

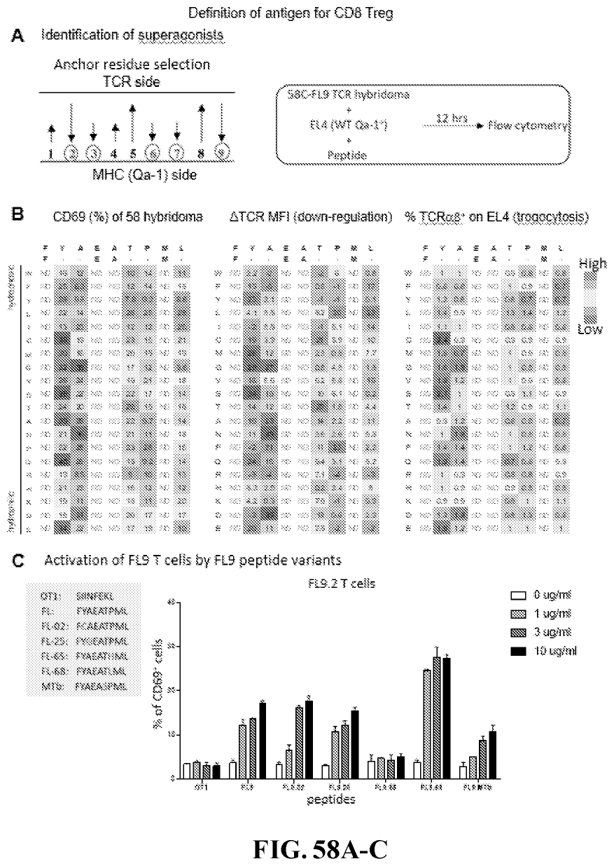

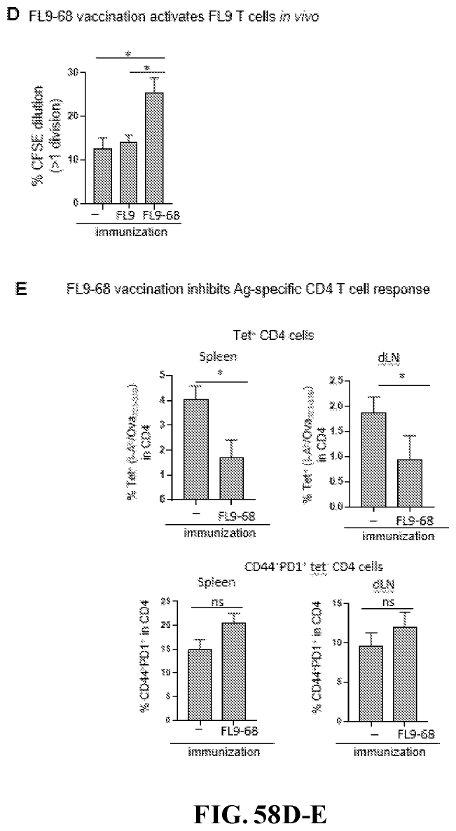

Immunization of mice with the FL9 self-peptide did not elicit detectable expansion of CD8 Treg (FIG. 57), perhaps reflecting the relatively low Qa-1 binding affinity and consequent weak TCR activation of self peptides (Kambayashi et al., 2004; Nakagawa et al., 2018; van Hall et al., 2010). We reasoned that efficient mobilization of Qa-1 restricted CD8 Treg-specific for self-peptides might require immunization with peptide analogs with increased Qa-1 binding activity. To systematically improve binding stability to Qa-1, we screened a peptide library composed of ˜100 aa-exchange variants of FL9 peptide at MHC-anchoring positions 2, 3, 6, 7 and 9 (FIG. 58A). The FL9 TCR⁺ 58C hybridoma was incubated with EL4 (Qa-1⁺) cells that had been pulsed with FL9 peptide variants and monitored for CD69 upregulation and TCR downregulation by 58C cells in order to define stimulatory activity of each peptide variant (FIG. 58B, left and middle panels). The interaction of Qa-1-peptide complexes with the FL9 TCR was also evaluated by measurement of TCR trogocytosis, which reports the strength of TCR binding to defined pMHC ligands (Li et al., 2019) (FIG. 58B, right). Variant FL9 peptides that stimulated increased FL9 TCR⁺ hybridoma responses compared with the native FL9 peptide (as judged by CD69 expression, TCR downregulation and increased trogocytosis) were then subjected to dose-response analysis.

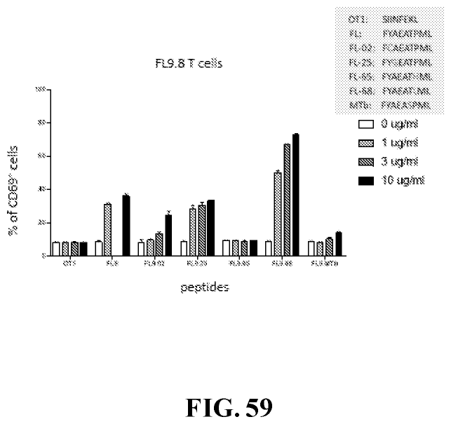

This analysis revealed that a FL9 peptide variant containing a P→L substitution at pos 7—termed FL9-68—displayed markedly enhanced dose-dependent stimulatory activity for FL9.2 and FL9.8 TCRs compared with the cognate FL9 self-peptide (FIGS. 58C, 59). Immunization with FL9-68 agonist peptide also activated FL9 TCR⁺ CD8 T cells after transfer into congeneic (CD45.1⁺ B6) hosts or TCRα^{-/-} hosts compared with native FL9 peptide (FIG. 58D). Immunization of CD45.1⁺ B6 hosts with Ova₃₂₃₋₃₃₉ in CFA followed by analysis of activated CD4 T cells revealed that FL9-68 vaccination inhibited the response of I-Ab/Ova₃₂₃₋₃₃₉ tet⁺ CD4 cells by 50% but did not reduce the numbers or percent of non-specifically activated CD4 cells that did not bind to the FL9-Qa-1 tetramer (FIG. 58E). Collectively, these findings indicate that FL9-specific CD8 Treg preferentially suppress Qa-1^{hi} CD4 cells that express high avidity TCR for the immunizing antigen without generalized immune suppression. These findings indicated that the FL9-68 peptide analog could be used to deliberately mobilize CD8 Treg.

### Peptide-Dependent Mobilization of CD8 Treg and Inhibition of Allo-Immunity

The observation that CD8 Treg mainly target high affinity CD4 cells indicated that mobilization of CD8 Treg may allow suppression of destructive autoimmune- or allo-responses without generalized immune suppression and the concomitant risk of increased vulnerability to pathogenic infection. Antibody-mediated rejection (AMR) remains a major barrier to successful solid organ transplantation. Since pathogenic alloantibodies mediating AMR are produced mainly by GC B cells after induction by Tfh cells (Kwun et al., 2017), increased expression of the Qa-1-FL9 complex by activated Tfh cells may allow targeting and suppression of pathogenic CD4 cells by Ag-specific CD8 Treg. Indeed, our recent analysis of the allograft response of B6.Qa-1 mutant (B6.Qa-1-D227K) mice indicated that disruption of the interaction between CD8 Treg and Qa-1 in recipients of a fully allogenic heart transplant model results in unchecked Tfh cell proliferation and accelerated Ab-mediated allograft injury (Choi et al., 2020).

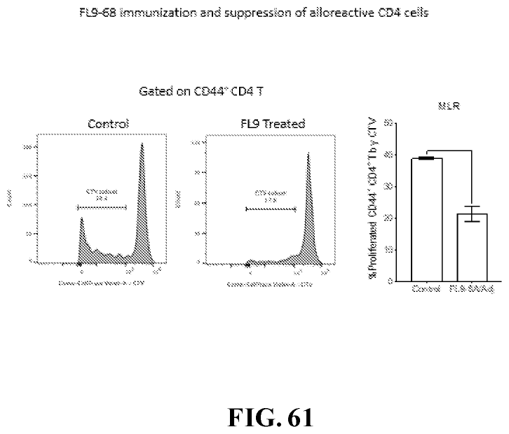

To test the ability of FL9-specific CD8 Treg to inhibit the anti-allograft response, we first determined whether SA peptide-dependent expansion of FL9-specific Tg T cells might suppress the anti-graft response. B6 mice bearing Balb/C skin given FL9 T cells together with FL9-68 peptide SA displayed a 4-5-fold increase in FL9-specific CD8 cells within the Ly49⁺ CD8 T cell pool (FIG. 60A). Ten days after transplant with Balb/C heart graft, secondary lymphoid tissues revealed significantly reduced GC responses in hosts receiving FL9-68/IFA but not IFA alone, along with reduced numbers of Tfh cells (PD-1⁺CXCR5⁺ CD4⁺), activated GC B cells (FAS+GL⁻7⁺B220⁺), and plasma cells (B220-CD138⁺) (FIG. 60B). Suppression of the GC response by expanded FL9 T cells was associated with upregulation of Qa-1 by Tfh cells in graft recipients (FIG. 60C). Moreover, reduced GC responses were accompanied by reduced donor-specific antibody (DSA) responses and markedly diminished graft pathology, as measured by C4d deposition and level of immune cell infiltration (FIGS. 60D, E). The impact of CD8 Treg mobilization by SA peptide was also indicated by significantly prolonged graft survival after SA peptide vaccination compared to adjuvant alone (FIG. 60F), which is associated with a decrease in alloreactive activated CD4 cells (FIG. 61). These data indicate that peptide-based mobilization of CD8 Treg that recognize pathogenic CD4 cells via TCR-Qa-1-peptide interaction represents a viable therapeutic strategy for reduction of Ab-mediated injury reduction in a murine model of Ab-mediated rejection of heart allografts.

### Discussion

There is increasing evidence that the contribution of CD8 Treg to suppression of pathogenic host responses depends on specific recognition of MHC class Ia and Ib-peptide complexes expressed by activated CD4 effector cells (Kim et al., 2010; Nakagawa et al., 2018; Saligrama et al., 2019). However, we do not understand the relationship of these CD8 Treg to other members of this CD8 T cell subset and target pQa-1 and the relationship of these Qa-1-restricted CD8 Treg to CD8 Treg that recognize class Ia pMHC. Characterization of TCRs expressed by class Ib Qa-1-restricted CD8 Treg revealed preferential usage of TRAV and TRBV genes independent of their specificity for two structurally-distinct self-peptides. The highly conserved expression of CDR1/CDR2 regions that may interact with Qa-1 class Ib MHC molecules may dominate the TCR interaction with pMHC and allow Qa-1-restricted CD8 T cells specific for diverse self-peptides to escape peptide-mediated negative selection. This Qa-1-centric focus may also equip them to survey CD4 T cells with highly avid TCRs for immunizing Ag in peripheral lymphoid tissues.

Preferential TCR usage by clonal CD8 Treg specific for self-peptides was also apparent in polyclonal Qa-1-restricted CD8 Treg. Indeed, virtually all Qa-1-restricted CD8 Treg in the polyclonal CD8 T cell pool expressed Vα3.2/V5.1, since this Treg subset was dramatically reduced in Qa-1-deficient mice in contrast to triad⁺ Vα3.2/Vβ5.1⁻ CD8 cells. Definition of canonical TCR pairs specific for CD8 Treg in mice (and potential in HLA-E-restricted CD8 Treg in man) can allow for selective activation or deletion of these MHC-E-restricted CD8 Treg by appropriate antibodies specific for these TCR.

### TCR

We analyzed TCR Tg mice to define the development and function of CD8 Treg specific for the FL9-Qa-1 complex. Expression of Qa-1 was essential for both early thymic development and for later survival in peripheral tissues. Residual FL9-TCR⁺ CD8 T cells that developed in the absence of Qa-1 displayed markedly reduced survival and impaired activation in adoptive environments that expressed a WT Qa-1 phenotype (FIG. 51C, 51E). Since the Qa-1-dependence of this TCR⁺ subset was also apparent from analyses of polyclonal CD8 Treg in peripheral lymphoid tissues, these data indicate that the TCR recognition bias towards Qa-1 imposed by TRAV9 and TRBV12 gene expression and highly-conserved CDR1/2 may allow these self-peptide-specific CD8 T cells to undergo successful escape from negative selection and survey activated T cells for increased expression of pQa-1.

### Antigens

Expression of the Qa-1-FL9 complex and a substantial fraction of CD4 cells may allow sensitive monitoring of increased pQa-1 by Ag-activated but not non-specifically activated CD4 T to stimulate CD8 Treg in vitro and allowed targeting of CD8 Treg in vivo. In vivo analysis revealed that suppressive activity depended on specific recognition and elimination of relatively high avidity CD4 T cells for cognate Ag. More than 90% of relatively high avidity tetramer* CD4 T cells were eliminated, while non-specifically activated tetramer-negative CD4 cells were spared. This may reflect robust upregulation of Qa-1 by tet+CD4 T cells (with relatively high avidity for cognate Ag), allowing efficient targeting of the major source of helper function and associated B-cell-dependent Ab responses without generalized immune suppression (Fazilleau et al., 2009; Tubo et al., 2013).

### Development

Analysis of T cells from FL9 TCR Tg mice also revealed that CD8 Treg express low levels of CD8 and TCR, reflecting their self-reactivity (FIG. 50C), and are maintained via continuous recognition of Qa-1. Since self-reactive CD8 Treg can be significantly more anergic than T cells specific for foreign antigens, they may require a more potent TCR stimulation for efficient activation and expansion by agonistic peptide analogs (Yu et al., 2015; Yu et al., 2004). We screened APC peptides with altered residues at Qa-1 anchoring positions to identify agonists with enhanced binding to pMHCI and increased immunogenicity. The FL9-68 peptide variant that includes a P→L amino acid exchange at position 7 of the cognate FL9 self-peptide displayed significantly enhanced stimulating activity in vitro and in vivo. We found that activation of CD8 Treg by FL9-68 peptide encounter resulted in CD8 Treg expansion, reduced Tfh cell and GC B cell responses and diminished production of anti-graft Abs in a heart transplantation model (FIG. 60A-F). Activation and expantion of clonal CD8 Treg by superagonist peptides in organ transplant hosts might efficiently inhibit anti-graft Ab production and graft tissue damage.

Multiple autoimmune diseases have been associated with autoantibody generation secondary to dysregulated high affinity Tfh expansion (Kim et al., 2011; Mishra et al., 2021; Serr and Daniel, 2018). CD8 Treg-mediated control of autoAb generation is an essential mechanism for inhibition of autoimmune disease development (Nakagawa et al., 2018). In contrast to CD4 Treg, CD8 Treg can be expanded and activated in a pMHC-specific fashion and may efficiently target CD4 Th cells with high affinity for cognate antigens, including self-Ags that upregulate pQa-1 complexes on their surface. While we have shown a peptide-dependent strategy to stimulate CD8 Treg, characteristic TCR Va/V usage by CD8 Treg defined in this study might also be exploited for activation and expansion of CD8 Treg in vivo. Activation of CD8 Treg via anti-TRAV Abs (targeting conserved CDR1/CDR2) may mobilize of a broad repertoire of CD8 Treg to allow efficient suppression of pQa-1 pathogenic CD4 cells. The efficacy of CD8 Treg expansion followed by inhibiton of autoAb generation and accompanying pathology can be tested in mouse models of autoimmune disease, including EAE, TlD (NOD) and SLE (BXSB-Yaa). Expression of the nonclassical MHC gene products (MHC-E) in murine (Qa-1) models and in humans (HLA-E) are both limited to two alleles, unlike the highly polymorphic classical MHC genes (Nakagawa et al., 2018). It is reasonable to speculate that HLA-E-restricted CD8 Treg may also express a limited TRAV and TRBV repertoire. Identification of homologous TCR expressed by human CD8 Treg may allow selective mobilization of HLA-E-restricted human CD8 Treg as an attractive strategy for the treatment of antibody-mediated pathologic conditions.

### Methods

Mice

C57BL/6 (B6), B6.SJL-PtprcaPepcb/BoyJ (B6.CD45.1), Balb/C, C57BL/6-Tg(TcraTcrb)1100Mjb/J (OT-1), B6.129P2-H2-K1t^{mlB}peH2-D1tml^{B}pe/DcrJ (Kb^{-/-}Db^{-/-}), C57BL6-Tg(Ins2-TFRC/OVA)296Wehi/WehiJ (Rip-mOVA), and B6.129S2-TCRa^{mlMom}/J (TCRa^{-/-}) mice were obtained from the Jackson laboratory (Bar Harbor ME). B6.Qa-1.D227K KI and B6.Qa-1^{-/-} (B6.129S6-H2-T23^{tmlcat}/J) mice were generated in the laboratory and previously described (Hu et al., 2004; Kim et al., 2010; Lu et al., 2007). FL9.2, FL9.8 TCR Tg mice were generated in the laboratory as described below and maintained on a Qa-1 WT and KO background. ERAAP^{-/-} mice were provided by Dr. Kenneth Rock (UMASS Medical Center, Worcester). All experiments were performed in compliance with institutional guidelines as approved by the Animal Care and Use Committee of the Dana-Farber Cancer Institute (DFCI).

### Antibodies and Flow Cytometry

Fluorescence-labelled antibodies for TCRβ (clone: H57-597), CD3ε (17A2), CD44 (IM7), CD122 (TM-β1), Ly49C/I/F/H (14B11), Vα3.2 (RR3-16), Vα2 (B20.1), Vβ5.1/5.2 (MR9-4), CD4 (RM4-5), CD8α (53-6.7), CD8β (YTS156.7.7), CD69 (H1.2F3), Qa-1b (6A8.6F10.1A6), PD-1 (29F.1A12), Act. Caspase 3 (5A1E), Ki67 (16A.8), B220 (RA3-6B2), Fas (SA367H8), CXCR5 (SPRCL5), FoxP3 (FJK-16S), NKG2D (A10) and NKG2A (20d5) were purchased from BD Biosciences, eBioscience and Biolegend. For the detection of FL9 T cells, Qa-1/FL9-PE, Qalb/FL9-APC, Qa-1b/Hsp60p216-PE, Qa-1b/Hsp60p216-APC tetramers were generated by NIH tetranmer core facility and provided for this study. I-Ab/Ova₃₂₃₋₃₃₉ tetramers were purchased from MBL International.

### Identification and Isolation of FL9 Specific TCR

Bone marrow derived DCs were generated from Kb^{-/-}Db^{-/-} mice in the presence of 20 ng/ml GM-CSF. 6 days later, DCs were stimulated with 50 ng/ml LPS for 12 hrs. DCs were irradiated (30 Gy) and pulsed with FL9 peptide by incubating with 10 μg/ml FL9 peptides for 2 hrs at 37° C. FL9-loaded Kb^{-/-}Db^{-/-} DCs were injected into WT B6 mice at day 0, 8 and 15. At day 22, Qa-1b/FL9 Tet⁺ cells were detected in the CD44⁺ CD122⁺Ly49⁺ CD8 subset and single Tet⁺ cells sorted by FACS. Identification of TCRa and TCRb chains for each sorted cells was performed according to a previously published protocol (Hamana et al., 2016). In brief, one-step RT-PCR was performed by adding RT-PCR mix to each well. Primers for the RT-PCR mix include the leader sequences and constant region sequences of TCRs where adapter sequences were added to the 5′ end of the leader primers (Hamana et al., 2016). cDNA from this RT-PCR was used to amplify TCRa and TCRb separately using the nested PCR principle. The PCR products were the sequenced using mTRAC_1st2R and mTRBC_1st2R primers for TCRα and TCRβ amplicons respectively and analyzed with the IMGT/V-Quest algorithm (http://www.imgt.org).

### Generation of TCR⁺ Hybridoma and TCR Affinity Test

The cDNAs encoding the TCRα and TCRβ chain were inserted into the pMIG vector that contains GFP cassette, which was transfected into the PLAT-E cells using FuGENE6 (Promega). The culture medium was replaced with the fresh medium in 24 hrs and supernatant was collected 72 hrs after transfection and used to transduce TCR^{-/-} 58C hybridoma. Expression of TCRα and TCRβ pairs on the surface of 58C hybridoma was analyzed by staining with Qa-1b/FL9 tetramers, anti-CD3$ and anti-TCR Vβ Abs.

Relative affinity of FL9.8 and FL9.2 TCR was analyzed by measuring the tetramer staining decay kinetics (Savage et al., 1999). FL9.8 TCR⁺ and FL9.2 TCR⁺ hybriboma were incubated with PE conjugated Qa-1b/FL9 tetramers in the presence of anti-Qa-1 Abs. Cells were fixed at different time points (0-120 min) after initiation of incubation and the intensity of PE staining was measured as an indication of tetramer binding by flowcytometry.

### Generation of FL9 TCR Tg Mice

FL9.2 and FL9.8 TCR transgenes were generated by replacing the TCR V(D)J elements of the pES.42.1c and pKS913.CD18.31 vectors that have been used previously to generate OT-I TCR Tg mice (Hogquist et al., 1994) with each TCRα and TCRP cDNAs fragments for FL9.2 and FL9.8 TCRs. The vector was linearized and used to target C57BL/6 ES cells using standard methods at the Transgenic Core Facility at Beth Israel Deaconess Medical Center. Founder lines for the FL9.2 and FL9.8 TCR Tg mice were established after genotyping with the following primers: common primer set for both FL9.2 and FL9.8 TCRα5′-CTAGAAGACTCAGGGTCTGA-3′ and 5′-TCGGCACATTGATTTGGGAGTCA-3′ amplified 1kbp for the transgene, a primer set for FL9.2 TCRb 5′-ACACTGTCCTCGCTGATTCTG-3′ and 5′-GATGTGAATCTTACCGAGAACAGTCAGTCTGGTTC-3′ and a primer set for FL9.8 TCRQ 5′-TAACACTGTCCTCGCTGAC-3′ and ATACAGCGTTTCTGCACTAG-3′ both amplified 500 bp for transgene.

### Peptide Mutageneisis and Superagonist Peptide Screen

A peptide library was generated by single mutation of each Qa-1 anchoring position (p 2, 3, 6, 7 and 9) of FL9 peptide (FYAEATPML) with 20 aa, which is composed of 96 FL9 variant peptides. FL9.8 TCR⁺ 58C hybridoma were incubated with EL4 cells that were pulsed with each FL9 variant. After 12 hrs, CD69 expression and level of TCR expression was measured by flowcytometry. For the analysis of the binding strength of FL9 TCR with Qa-1-FL9 variants, trogocytosis was measured directly by the detection of FL9 TCR (Vα3.2⁺Vβ5⁺) on EL4 cells. FL9.8 TCR⁺ 58C hybridoma were co-cultured with EL4 cells that were pulsed with FL9 variant peptides from the library. After 2 hrs, percentage of Vα3.2⁺V35⁺EL4 cells were assessed by flowcytometry as a measurement of trogocytosis.

### Adoptive Transfer and In Vivo Suppression Assay

Qa-1.WT or Qa-1.D227K KI mice were i.p. immunized with 100 μg Ova₃₂₃₋₃₃₉ peptides in CFA. 7 days later, 1×10⁵ CD25⁻ CD4 cells were isolated from these mice and transferred into WT B6 mice along with FL9 Tg T cells. WT B6 adoptive hosts were immunized on footpad with 20 μg Ova₃₂₃₋₃₃₉ peptide in CFA. After 7 days, the frequency and numbers of I-Ab/Ova³²³⁻³³⁹tetramer⁺ CD4 cells and activated CD4 cells were assessed in the inguinal and popliteal LNs of B6 hosts by flowcytometry.

### FL9 Peptide Immunization

CD45.1⁺ B6 or TCRα^{-/-} mice were transferred with CFSE labelled 2×10⁶ FL9.2 Tg T cells followed by i.p. immunization with 100 μg FL9 or FL9-68 peptides in CFA. Proliferation and activation of FL9.2 Tg T cells in the adoptive hosts were analzyed by assessing CFSE dilution, Ki67 and CD69 expression at day 3 and 6 after transfer.

### Heart Transplantation and Analysis of Immune Response

B6 mice were immunized i.p with 50 μg FL9-68/IFA or with IFA alone at day 0 and 7 followed by a BALB/C→B6 skin transplant at day 10. FL9-68/IFA or IFA immunization was repeated on days 10, 13 and 16. At day 27, fully vascularized Balb/C hearts were transplanted into the abdominal cavity of B6 mice using microsurgical techniques, as previously described (Cai et al., 2016). Heart graft survival was determined by monitoring palpable heart beating. At day X after skin sensitization, levels of FL9 T cells, Tfh, GC B and plasma cells in dLNs were analyzed by flowcytometry. Serum was collected from the heart graft recipient B6 mice that were either immunized with FL9-68/IFA or IFA alone at day 16. Serially diluted serum was incubated with 1×10⁶ donor splenocytes in total volume 100 μl PBS for 30 min followed by detection of surface bound Abs on CD4 cells using anti-CD4 (Biolegend, Clone RM4-5) and anti-mouse IgG1 Abs (BD Biosciences, Clone A85-1). Histological analysis of heart grafts was performed by InvivoEx company using anti-C4d Ab (Hycult Biotech) and Vector Blue Alkaline Phosphatase Substrate Kit (Vector Laboratories).

### Statistical Analysis

Prism v.9.0 was (GraphPad Software) was used for the statistical analyses. Statistical significance was calculated according to Wilcoxon-Mann-Whitney rank sum test for comparison of two conditions; Kruskal-Wallis test was performed for comparison of more than two conditions. A P value of <0.05 was considered to be statistically significant (* =<0.05, ** =<0.01, *** =<0.001, **** =<0.0001.

### REFERENCES

Cai, S., Ichimaru, N., Zhao, M., Fujino, M., Ito, H., Ota, U., Nakajima, M., Tanaka, T., Nonomura, N., Li, X. K., and Takahara, S. (2016). Prolonged Mouse Cardiac Graft Cold Storage via Attenuating Ischemia-Reperfusion Injury Using a New Antioxidant-Based Preservation Solution. Transplantation 100, 1032-1040. 10.1097/TP.0000000000001079.
Choi, J. Y., Eskandari, S. K., Cai, S., Sulkaj, I., Assaker, J. P., Allos, H., AlHaddad, J., Muhsin, S. A., Alhussain, E., Mansouri, A., et al. (2020). Regulatory CD8 T cells that recognize Qa-1 expressed by CD4 T-helper cells inhibit rejection of heart allografts. Proc Natl Acad Sci USA 117, 6042-6046. 10.1073/pnas.1918950117.
Dai, Z., Turtle, C. J., Booth, G. C., Riddell, S. R., Gooley, T. A., Stevens, A. M., Spies, T., and Groh, V. (2009). Normally occurring NKG2D+CD4⁺ T cells are immunosuppressive and inversely correlated with disease activity in juvenile-onset lupus. J Exp Med 206, 793-805. 10.1084/jem.20081648.
Dhanji, S., Teh, S. J., Oble, D., Priatel, J. J., and Teh, H. S. (2004). Self-reactive memory-phenotype CD8 T cells exhibit both MHC-restricted and non-MHC-restricted cytotoxicity: a role for the T-cell receptor and natural killer cell receptors. Blood 104, 2116-2123. 10.1182/blood-2004-01-0150.
Fazilleau, N., McHeyzer-Williams, L. J., Rosen, H., and McHeyzer-Williams, M. G. (2009). The function of follicular helper T cells is regulated by the strength of T cell antigen receptor binding. Nat Immunol 10, 375-384. 10.1038/ni.1704.
Hamana, H., Shitaoka, K., Kishi, H., Ozawa, T., and Muraguchi, A. (2016). A novel, rapid and efficient method of cloning functional antigen-specific T-cell receptors from single human and mouse T-cells. Biochem Biophys Res Commun 474, 709-714. 10.1016/j.bbrc.2016.05.015.
Hogquist, K. A., Jameson, S. C., Heath, W. R., Howard, J. L., Bevan, M. J., and Carbone, F. R. (1994). T cell receptor antagonist peptides induce positive selection. Cell 76, 17-27.
Hu, D., Ikizawa, K., Lu, L., Sanchirico, M. E., Shinohara, M. L., and Cantor, H. (2004). Analysis of regulatory CD8 T cells in Qa-1-deficient mice. Nat Immunol 5, 516-523.
Kambayashi, T., Kraft-Leavy, J. R., Dauner, J. G., Sullivan, B. A., Laur, O., and Jensen, P. E. (2004). The nonclassical MHC class I molecule Qa-1 forms unstable peptide complexes. J.Immunol 172, 1661-1669.
Kim, H. J., Verbinnen, B., Tang, X., Lu, L., and Cantor, H. (2010). Inhibition of follicular T helper cells by CD8⁺ Treg is essential for self tolerance Nature 467, 328-332.
Kim, H. J., Wang, X., Radfar, S., Sproule, T. J., Roopenian, D. C., and Cantor, H. (2011). CD8⁺ T regulatory cells express the Ly49 class I MHC receptor and are defective in autoimmune-prone B6-Yaa mice. Proc.Natl.Acad.Sci.U.S.A 108, 2010-2015.
Kwun, J., Manook, M., Page, E., Burghuber, C., Hong, J., and Knechtle, S. J. (2017). Crosstalk Between T and B Cells in the Germinal Center After Transplantation. Transplantation 101, 704-712. 10.1097/TP.0000000000001588.
Lazaro, E., Godfrey, S. B., Stamegna, P., Ogbechie, T., Kerrigan, C., Zhang, M., Walker, B. D., and Le Gall, S. (2009). Differential HIV epitope processing in monocytes and CD4 T cells affects cytotoxic T lymphocyte recognition. J Infect Dis 200, 236-243. 10.1086/599837.
Leavenworth, J. W., Tang, X., Kim, H. J., Wang, X., and Cantor, H. (2013).
Amelioration of arthritis through mobilization of peptide-specific CD8⁺ regulatory T cells Journal of Clinical Investigation 123, 1382-1389.
Li, G., Bethune, M. T., Wong, S., Joglekar, A. V., Leonard, M. T., Wang, J. K., Kim, J. T., Cheng, D., Peng, S., Zaretsky, J. M., et al. (2019). T cell antigen discovery via trogocytosis. Nat Methods 16, 183-190. 10.1038/s41592-018-0305-7.
Lu, L., Ikizawa, K., Hu, D., Werneck, M. B. F., Wucherpfennig, K. W., and Cantor, H. (2007). Regulation of activated CD4⁺ T cells by NK cells via the Qa-1-NKG2A pathway Immunity 26, 593-604.
Mishra, S., Liao, W., Liu, Y., Yang, M., Ma, C., Wu, H., Zhao, M., Zhang, X., Qiu, Y., Lu, Q., and Zhang, N. (2021). TGF-beta and Eomes control the homeostasis of CD8+regulatory T cells. J Exp Med 218. 10.1084/jem.20200030.
Nagarajan, N. A., Gonzalez, F., and Shastri, N. (2012). Nonclassical MHC class Ib-restricted cytotoxic T cells monitor antigen processing in the endoplasmic reticulum. Nat.Immunol. 13, 579-586.
Nakagawa, H., Wang, L., Cantor, H., and Kim, H. J. (2018). New Insights Into the Biology of CD8 Regulatory T Cells. Adv Immunol 140, 1-20. 10.1016/bs.ai.2018.09.001.
Saligrama, N., Zhao, F., Sikora, M. J., Serratelli, W. S., Fernandes, R. A., Louis, D. M., Yao, W., Ji, X., Idoyaga, J., Mahajan, V. B., et al. (2019). Opposing T cell responses in experimental autoimmune encephalomyelitis. Nature 572, 481-487. 10.1038/s41586-019-1467-x.
Savage, P. A., Boniface, J. J., and Davis, M. M. (1999). A kinetic basis for T cell receptor repertoire selection during an immune response. Immunity 10, 485-492. 10.1016/s1074-7613(00)80048-5.
Schonrich, G., Kalinke, U., Momburg, F., Malissen, M., Schmitt-Verhulst, A. M., Malissen, B., Hammerling, G. J., and Arnold, B. (1991). Down-regulation of T cell receptors on self-reactive T cells as a novel mechanism for extrathymic tolerance induction. Cell 65, 293-304. 10.1016/0092-8674(91)90163-s.
Serr, I., and Daniel, C. (2018). Regulation of T Follicular Helper Cells in Islet Autoimmunity. Front Immunol 9, 1729. 10.3389/fimmu.2018.01729.
Tubo, N. J., Pagan, A. J., Taylor, J. J., Nelson, R. W., Linehan, J. L., Ertelt, J. M., Huseby, E. S., Way, S. S., and Jenkins, M. K. (2013). Single naïve CD4⁺ T cells from a diverse repertoire produce different effector cell types during infection. Cell 153, 785-796. 10.1016/j.cell.2013.04.007.
van Hall, T., Oliveira, C. C., Joosten, S. A., and Ottenhoff, T. H. (2010). The other Janus face of Qa-1 and HLA-E: diverse peptide repertoires in times of stress Microbes Infect 12, 910-918.
Vivier, E., and Anfossi, N. (2004). Inhibitory NK-cell receptors on T cells: witness of the past, actors of the future. Nat.Rev.Immunol 4, 190-198.
Xiao, Z., Mescher, M. F., and Jameson, S. C. (2007). Detuning CD8 T cells: down-regulation of CD8 expression, tetramer binding, and response during CTL activation. J Exp Med 204, 2667-2677. 10.1084/jem.20062376.
Yu, W., Jiang, N., Ebert, P. J., Kidd, B. A., Muller, S., Lund, P. J., Juang, J., Adachi, K., Tse, T., Bimbaum, M. E., et al. (2015). Clonal Deletion Prunes but Does Not Eliminate Self-Specific alphabeta CD8(+) T Lymphocytes. Immunity 42, 929-941. 10.1016/j.immuni.2015.05.001.
Yu, Z., Theoret, M. R., Touloukian, C. E., Surman, D. R., Garman, S. C., Feigenbaum, L., Baxter, T. K., Baker, B. M., and Restifo, N. P. (2004). Poor immunogenicity of a self/tumor antigen derives from peptide-MHC-I instability and is independent of tolerance. J Clin Invest 114, 551-559. 10.1172/JCI21695.
Zloza, A., Lyons, G. E., Chlewicki, L. K., Kohlhapp, F. J., O'Sullivan, J. A., Lacek, A. T., Moore, T. V., Jagoda, M. C., Kumar, V., and Guevara-Patino, J. A. (2011). Engagement of NK receptor NKG2D, but not 2B4, results in self-reactive CD8⁺ T cells and autoimmune vitiligo. Autoimmunity 44, 599-606. 10.3109/08916934.2011.593599.

### Example 7

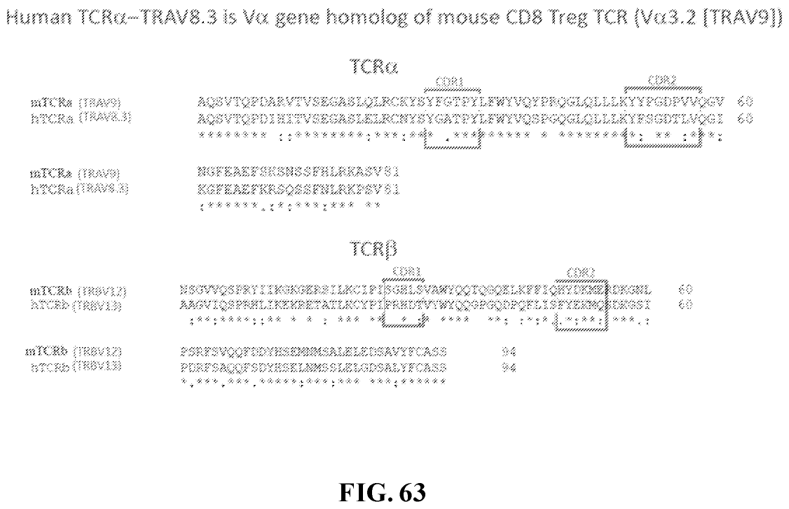

FIG. 63 A human TCRα sequence was identified that shows significant homology with mouse TCRa3.2 that is preferentially expressed by mouse CD8 Treg. Human TRAV8.3 V gene shows the highest level of similarity with mouse TRAV9 (Vα3.2). This in silico data will be verified with TCR sequences that will be obtained from KIR+HLA-E/FL9 tet+CD8 T cells.

### Example 8

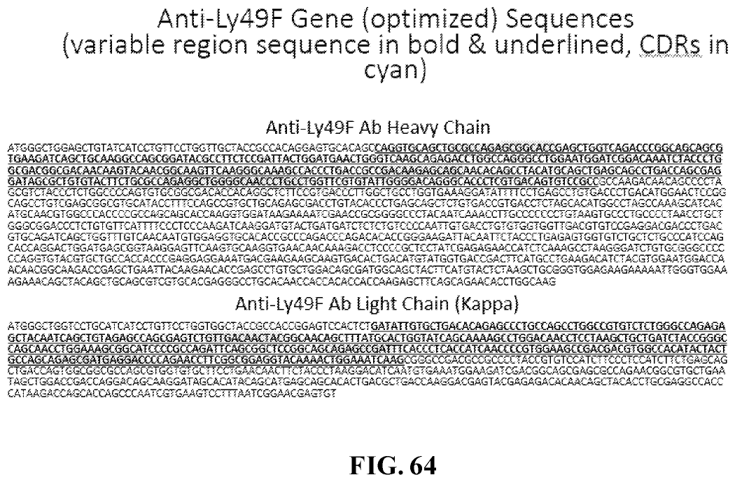

FIG. 64 DNA sequences encoding the heavy and light chain of anti-Ly49F Ab are shown. CDR1, 2 and 3 for heavy and light chains are marked in green (shaded).

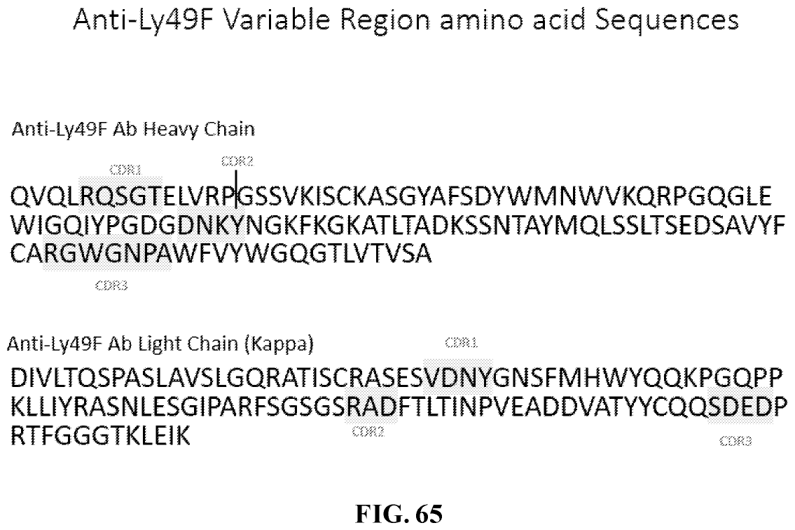

FIG. 65 Amino acid sequences for the heavy and light chains of anti-Ly49F Ab are shown. CDR1, 2, and 3 for heavy and light chains are marked in green (shaded).

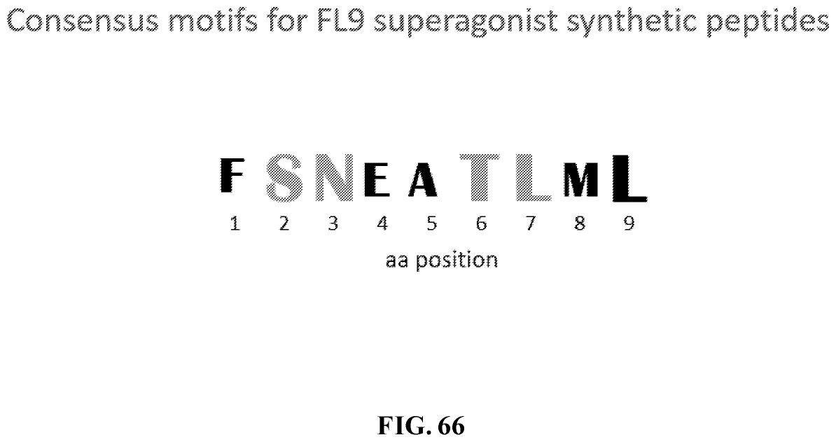

FIG. 66 Amino acids at each position of FL9 peptide variants that resulted in the highest FL9 T cell stimulatory capacity are selected and assembled as a candidate amino acid that may display superagonistic activity.

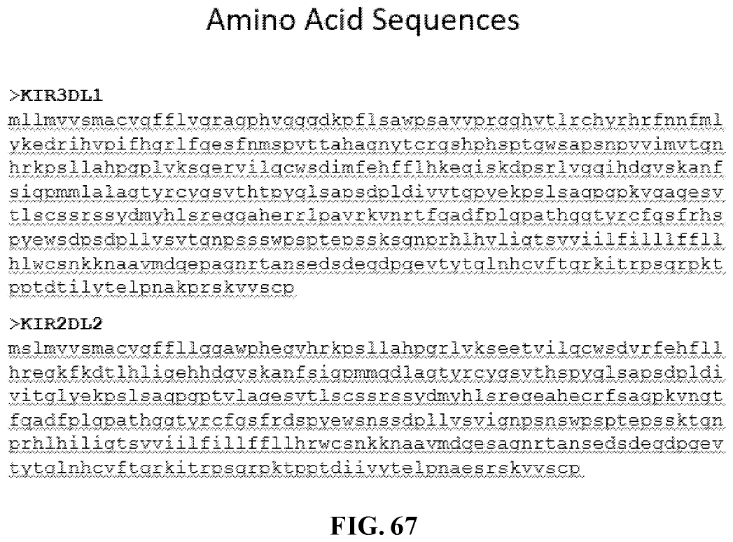

FIG. 67 Human CD8 Treg express subsets of KIR (Killer-cell immunoglobulin like receptor) -KIR3DL1, KIR2DL2, KIR2DL3-that can be used to identify these cells. KIR receptors are named based on the number of their extracellular Ig-like domains (2D or 3D) and by the length of their cytoplasmic tail (long (L), short (S). Amino acid sequences for KIR3DL1 and KIR2DL2 are shown.

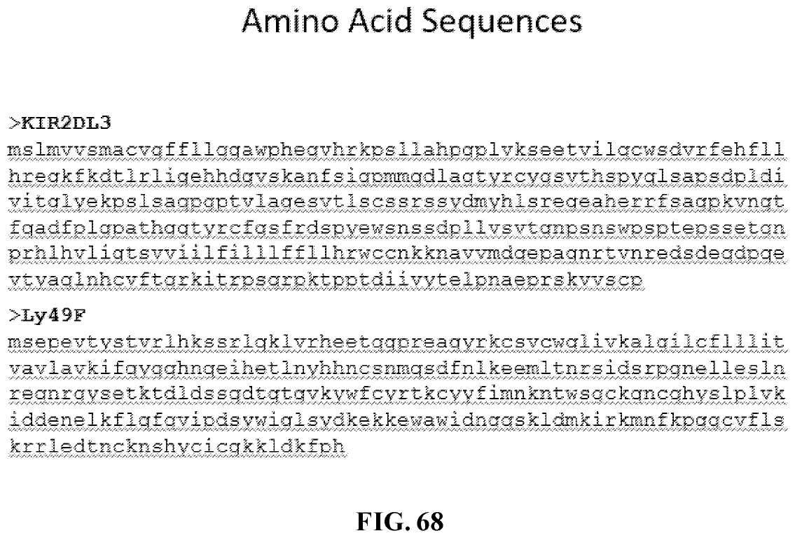

FIG. 68 Full length amino acid sequences for KIR2DL3 and Ly49F (mouse CD8 Treg marker) are shown.

### Example 9

FIG. 69 Release of immune system from CD8 Treg mediated suppression can enhance anti-tumor immune responses. Vaccination with modified MC38 cells (harboring increased immunogenicity) enhance overall immune responses, which may be accompanied by upregulation of Qa-1 on tumor-infiltrating immune cells. Anti-Ly49F Ab treatment (to deplete CD8 Treg) can be combined with a tumor cell vaccine, which leads to almost complete inhibition of tumor growth. Chimeric anti-Ly49F Ab was generated by replacing the mIgG1 Fc portion of anti-Ly49F Ab (HBF-719) with mIgG2a that efficiently depletes Ly49F+CD8 Treg in vivo.

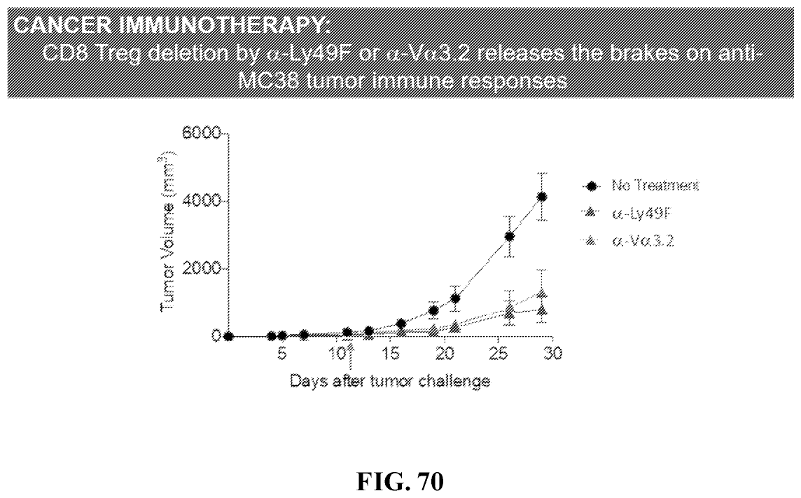

FIG. 70 shows cancer and CD8 Treg depletion. Tumor growth in B6 mice is shown that is inoculated with MC38 cells and treated with anti-Ly49 or anti-Vα3.2 Abs.

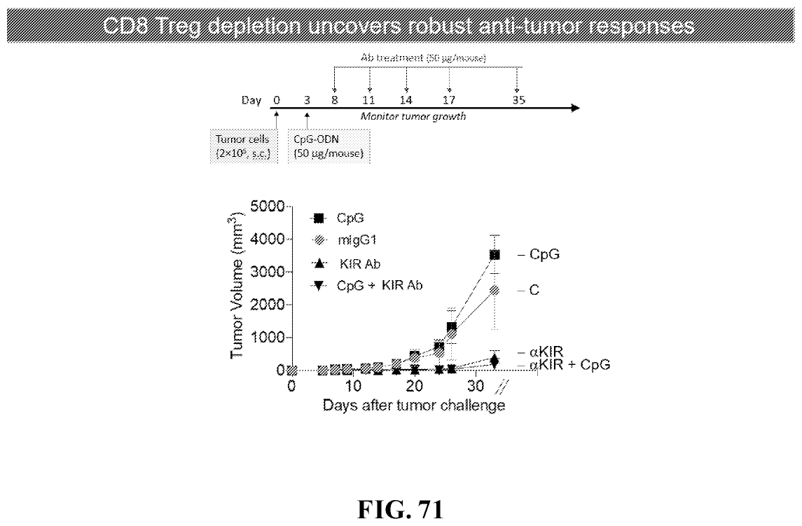

FIG. 71 depicts the impact of CD8 Treg depletion on anti-tumor immunity under conditions that elicit a Thl-biased immune response was tested by injection of CpG-ODN 3 days after inoculation of MC38 cells. Although treatment with CpG-ODN did not reveal a significant therapeutic effect, CD8 Treg depletion at day 8-10 either alone or with CpG-ODN treatment strongly inhibited tumor growth. These data indicate that strategies that target CD8 Treg-dependent immune suppression may represent an effective approach, as monotherapy or in combination with other immunotherapies, to enhance the immune response against cancers.

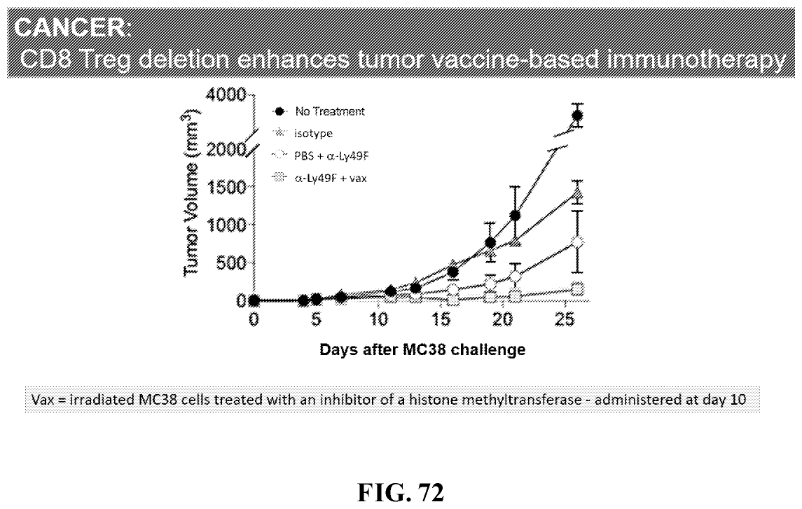

FIG. 72 depicts cancer and CD8 Treg depletion allowing for tumor vaccine therapy.

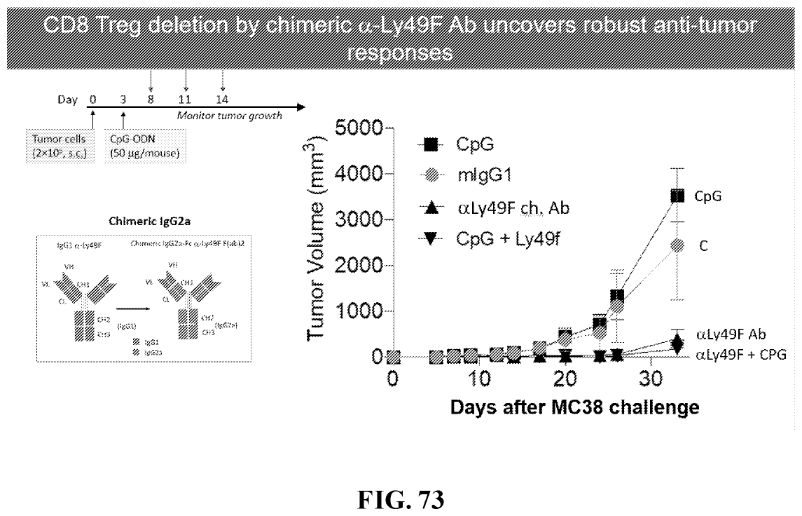

FIG. 73 depicts CD8 Treg depletion uncovering robust anti-tumor response.

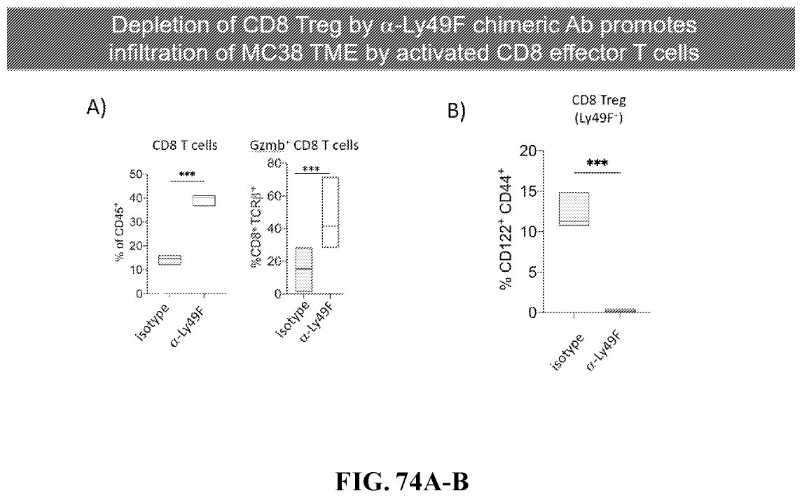

FIG. 74A-B shows the CD8 T cell profile in tumorsd grown in mice that were treated with isotype or anti-Ly49F Abs. Percent of CD8 T cells among CD45⁺ cells and expression of GzmB in CD8 T cells (A) and percentage of CD8 Treg (CD44⁺ CD122⁺Ly49⁺) in the CD45⁺ cells (B) are shown.

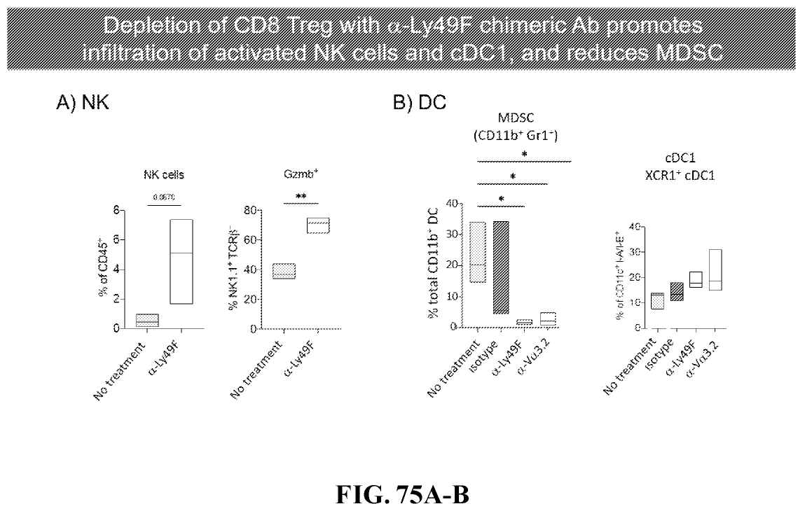

FIG. 75A-B shows NK and DC profile within the tumors grown in mice that were treated with isotype or anti-Ly49F Abs. (A) NK cell percentage among CD45⁺ cells and GzmB expression by NK cells within tumors. (B) Percentage of MDSC within CD11b+cells and cDCs within CD11c+I-Ab+cells.

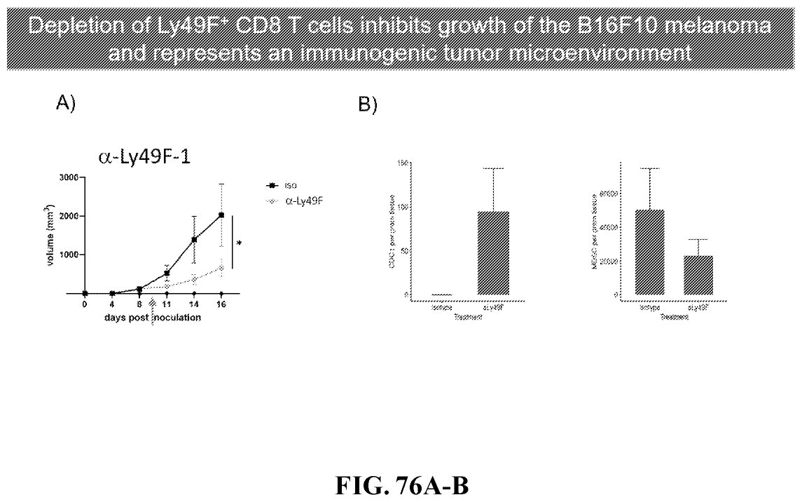

FIG. 76A-B illustrates the tumor growth in B6 mice that were inoculated with B16 melanoma and treated with anti-Ly49F Ab (A). Number of cDC and MDSC within tumors that were treated with isotype or anti-Ly49F Abs (B).

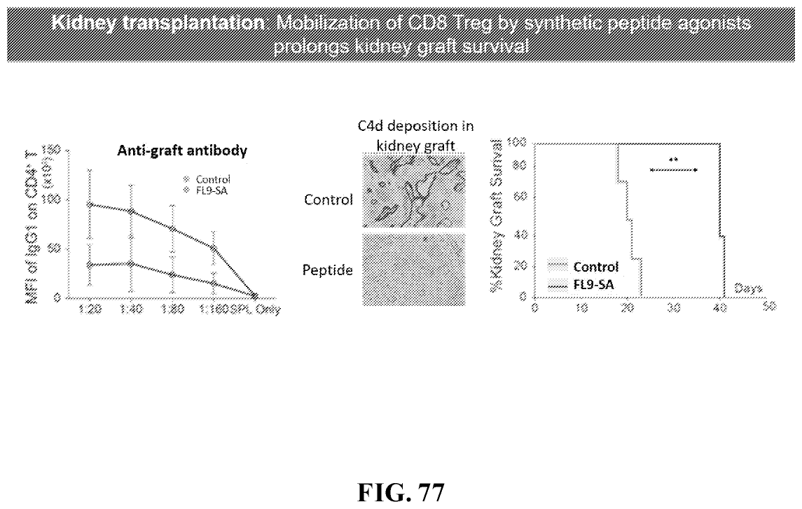

FIG. 77 The effect of peptide-superagonist (SA)-mediated expansion of CD8 Treg on anti-allograft immunity against fully-mismatched kidney transplants was also tested. Administration of FL9-68-IFA but not IFA alone markedly reduced donor specific Ab generation. Vaccination with FL9-SA significantly protected kidney grafts from antibody-mediated rejection, as judged by reduced C4d deposition in the peritubular capillaries of kidney allografts and substantial prolongation of allograft survival compared to controls (mean survival time: 20.5 vs. 40 days). These data indicate that peptide-based mobilization of CD8 Treg that recognize pathogenic CD4 Tfh cells represents a promising therapeutic approach for drug-free reduction of Ab-mediated injury in a murine model of and kidney allografts.

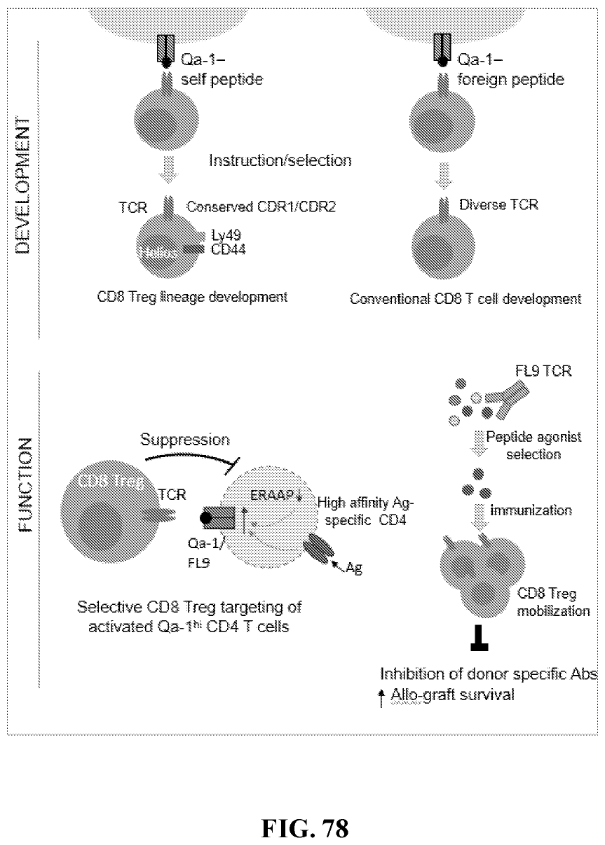

FIG. 78 Qa-1 restricted CD8 Treg develop in the thymus by recognition of Qa-1/peptide complex mainly by Qa-1 bidning of CDR1 and CDR2 of defined TCR (Vα3.2 in mice), which allows the devlopment of these selfreactove T cells without undergoing negative selection. Qa-1 restricted CD8 T cells with foreign reactivity may also develop, however, TCR repertoire of these CD8 T cells can be diverse and display a phenotype similar to MHC class Ia restricted conventional CD8 T cells. In the periphery, CD8 Treg recognize Qa-1/FL9 complex expressed by activated CD4 cells resulted from defective ERAAP function after Ag specific stimulation of the CD4 cells. TCR dependent targeting of Ag-activated CD4 cells allows selective suppression of Qa-1^{hi} CD4 cells without generalized immune suppression. Identification of peptide superagonists with the ability to stimulate FL9 T cells allows expansion/activation of CD8 Treg by peptide immunization. Peptide dependent mobilization of CD8 Treg can be applied to the pathogenic conditions in which Ab generation can cause fatal disease, including autoimmunity and Ab mediated organ rejection. For example, inhibition of donor specific Abs in the setting of heart and kidney transplantation allows prolonged graft survival in mice.

### Example 10

T Cell Receptor Usage Determines Thymic Differentiation and Function ofMHC Class Ib Restricted CD8⁺ Regulatory T Cells

### Abstract

Although most CD8⁺ T-cells are equipped to kill cells infected by microbial invaders, a subset may regulate immune responses. Murine and human CD8 regulatory activity is invested in a small (<5% CD8 cells) subset that express a characteristic triad of surface receptors—CD44, CD122 and Ly49/KIR, and eliminate activated CD4 T-cells through targeting of MHC class Ia or class Ib expressed by CD4⁺ T-helper cells. Here we characterize CD8 T regulatory cells (Treg) that target class Ib according to TCR expression, thymic-dependent development and regulatory function. Expression of TRAV9N3 and TRBV12-1/2 TCR genes that encode the Vα3.2/Vb5.1 TCR pair allows recognition and elimination of target cells that express Qa-1 associated with several distinct self-peptides, including FL9 and Hsp60-216. This interaction selectively elevates the high affinity CD4 T cell response and spares non-specifically activated CD4 cells, resulting in selective reduction of pathogenic antibody responses without generalized immune suppression.

Definition of TCR specific for Qa-1-FL9 allowed systematic mutagenesis of the FL9 self-peptide and identification of synthetic superagonist peptides that promote robust mobilization and expansion of CD8 Treg and efficient inhibition of Tfh-driven Ab responses to both conventional and transplantation antigens. Mobilization of CD8 Treg by agonist FL9 peptides in a preclinical model of MHC mismatched heart or kidney transplants reduced Tfh-driven allo-antibody responses and markedly prolonged organ graft survival. These insights into the TCR-based specificity of CD8 Treg and their peptide ligands open the way for new therapeutic approaches to dampen pathogenic Ab responses.

### Introduction

The immune system has evolved complex mechanisms that allow efficient destruction of microbial pathogens while sparing the host's own tissues. Maintenance of this balance depends, in part, on regulatory T cells. Although most CD8⁺ T-cells are equipped to kill cells infected by microbial invaders, there is increasing evidence that a subset of mouse and human CD8⁺ T-cells is genetically programmed to suppress immune responses¹⁻³ Murine and human CD8 regulatory activity are invested in a small (<5%) subset of CD8 T cells that expresses a characteristic triad of surface receptors: CD44, CD122 and Ly49/KIR²⁻⁴ that are equipped to mediate perforin-dependent killing of chronically-activated and autoreactive CD4 cells²,. Analysis of autoimmune disorders has revealed that CD8 T regulatory cells (CD8 Treg) inhibit pathogenic responses through recognition of self-peptides associated with MHC class Ia or class Ib (MHC-E: mouse Qa-1 and human HLA-E)^{1,2} expressed by target CD4⁺T-helper cells. Here we define class Ib-restricted CD8 Treg according to TCR expression, thymus-dependent development and specific recognition mechanisms that allow elimination of activated CD4 T cells that express appropriate Qa-1-self peptide complexes.

Cell surface expression of Qa-1-peptide complexes by activated T cells depends on trimming by several enzymes, including an endoplasmic reticulum aminopeptidase associated with antigen processing (ERAAP), which digests larger peptides into 9/10mers that efficiently bind to Qa-1. Shastri and colleagues showed that diminished or defective ERAAP activity associated with chronic activation of CD4 T cells is marked by increased expression of a Qa-1-associated self-peptide termed FL9 and an increase in FL9-specific memory CD8 T cells^{6,7}. Chronically-activated CD4 T cells also express self-peptides derived from the Hsp60 protein that associate with Qa-1 and mobilize CD8 Treg (Leavenworth et al, 2013). To define the TCRs used for recognition of these pQa-1 complexes, we cloned and analyzed two large sets of TCR expressed by CD8 Treg that recognize the two structurally-distinct self-peptides—FL9 and Hsp60-216—complexed to Qa-1^{7,8}. This analysis revealed enrichment of TRAV and TRBV genes encoding highly conserved CDR1 and CDR2 regions and highly variable relatively heterogenous CDR3 sequences associated with recognition of either self-peptide. Analysis of TCR transgenic CD8 T cells engaging these receptors confirmed a strong bias towards MHC (Qa-1) recognition that may allow CD8 Treg to escape negative selection by self-peptides and efficiently recognize and eliminate Qa-1⁺ CD4⁺ Th cells in peripheral tissues. Indeed, mutation or deletion of Qa-1 almost completely prevented intrathymic development, peripheral survival of CD8 Treg, and abrogated targeting and elimination of activated CD4 T cells. Moreover, since virtually all Qa-1-restricted CD8 Treg expressed this Vα3.2/Vβ5.1 pair, they were also able to abolish CD8 Treg activity by deletion of CD8 T cells that expressed the TCR Vα3.2/V5.1 pair.

Identification and expression of TCRs expressed by regulatory lineage of Qa-1-restricted CD8 T cells also allowed definition of synthetic variants of the FL9 self-peptide that efficiently mobilized CD8 Treg and suppressed pathogenic CD4 cells during immune responses. We used this approach to inhibit Tfh-driven allo-antibody responses and prolong surivial of heart and kidney allografts in preclinical murine models of organ transplantation. These insights into TCR-based specificity of CD8 Treg indicate new therapeutic approaches to dampen pathogenic or undesired Ab responses.

### Results

### Identification of TCRs Specific for Qa-1-Selfpeptide Complexes

Insight into the specialized function of both class Ia- and class Ib-restricted CD8 Treg has relied mainly on isolation of both subsets of CD8 Treg using a triad of shared surface markers—CD44, CD122 and Ly49. Here we distinguish class Ib (Qa-1-restricted) CD8 Treg from class Ia-restricted Treg according to expression of TCR specific for two structurally-unrelated self-peptides—FL9 and Hsp60, that are presented by Qa-1 and allow specific targeting of CD4 cells by CD8 Treg¹,7,8.

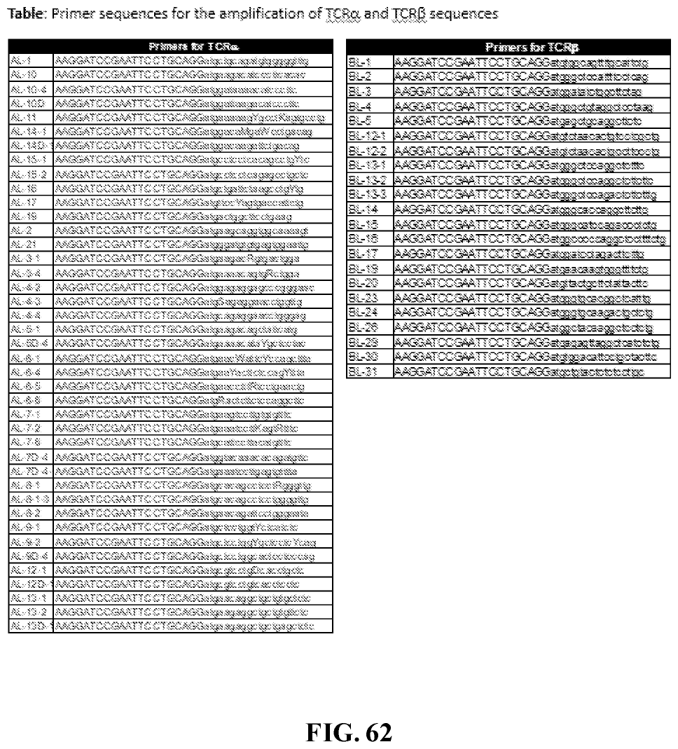

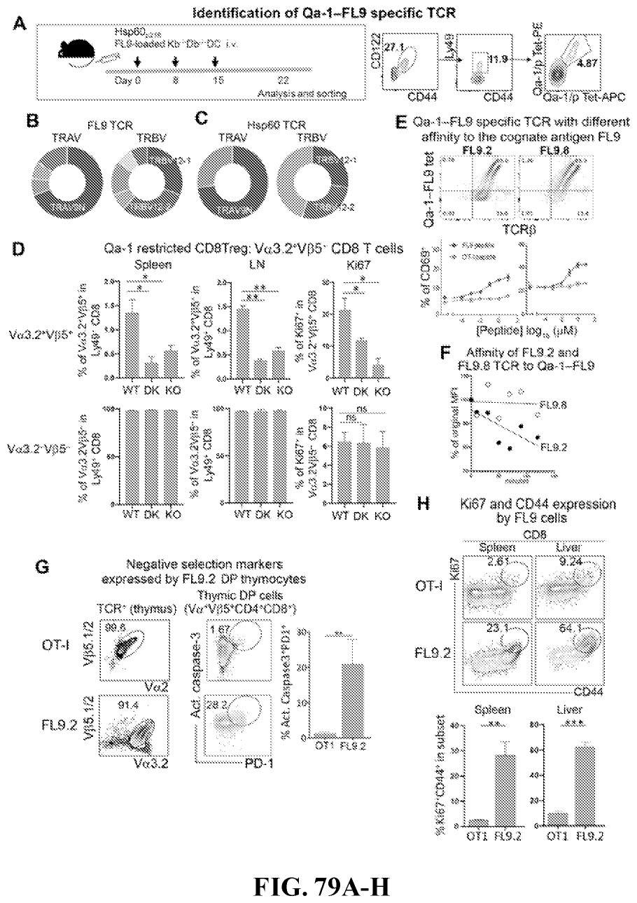

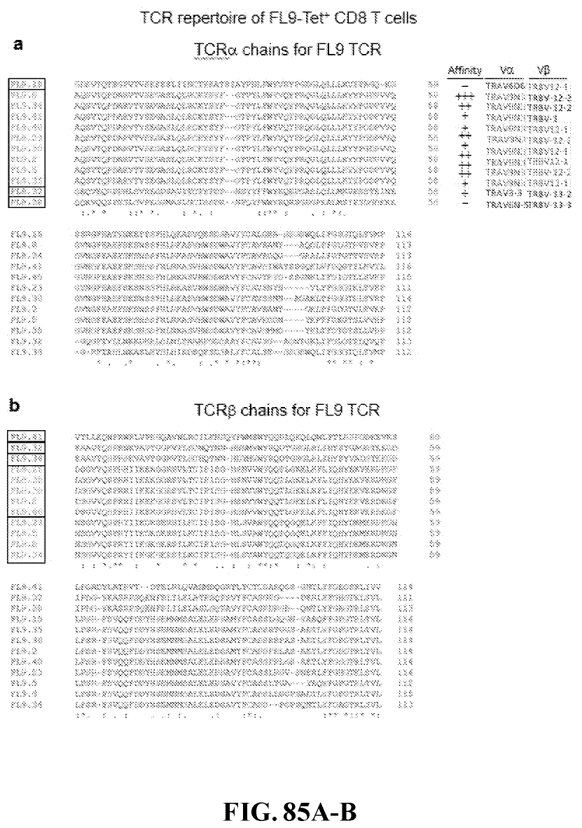

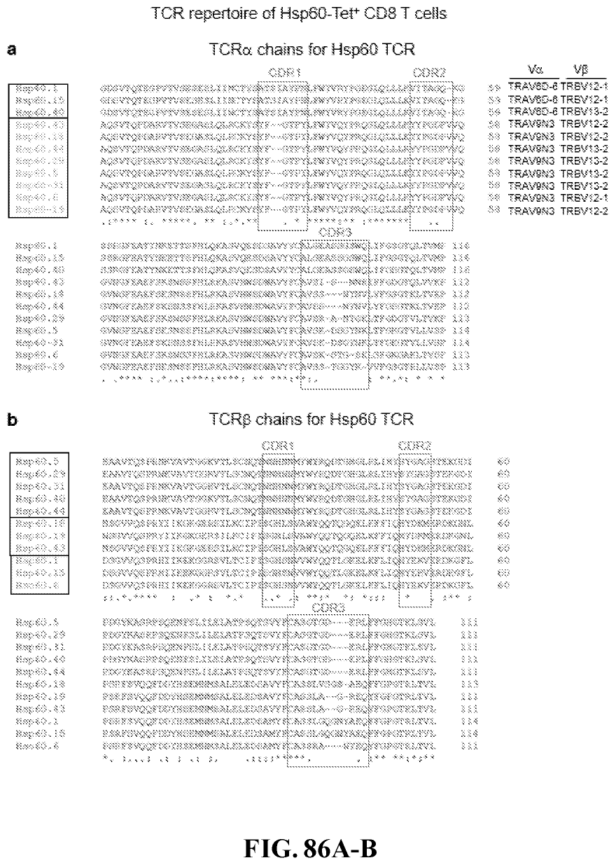

We used Qa-1-FL9 and Qa-1-Hsp60 peptide tetramers to detect, sort and analyze TCR expression by tetramer-positive (tet+) cells according to single-cell TCR sequencing (FIG. 79A, FIG. 62)). Analysis of paired TCRs from 12 independent Qa-1-FL9 tetramer-binding cells revealed that 9/12 TCR specific for Qa-1-FL9 tetramers expressed the TRAV9N3 gene (Vα3.2) and 9/12 expressed TRBV12-1/2 (Vβ5.1,2) (FIG. 79B, FIG. 85A-B). Analysis of 11 independent Qa-1-Hsp60-specific CD8 T cells revealed that 8/11 also expressed TRAV9N3/Vα3.2, and 6/11 expressed the TRBV 12-1/2/Vβ5.1,2 (FIG. 79C, FIG. 86A-B). Both TCR sets expressed nearly identical CDR1 and CDR2 sequences (which can represent MHC contact elements) but carry distinct peptide-specific CDR3 regions. Expression of TRAV9N3 and TRBV12-1/2 pair is essential for expression of high affinity FL9 T cells, since TCRs composed of non-TRAV9N3 or TRBV12-1/2 pairs display markedly reduced binding affinity to Qa-1-FL9 complexes (FIGS. 85A-B, 48-C). This conserved TCR repertoire used for recognition of two structurally-distinct self-peptides presented by Qa-1 indicates the contribution of an interaction between highly-conserved CDR1/CDR2 TCR regions with Qa-1 along with a second interaction between CDR3 and the FL9 and Hsp60 self-peptides.

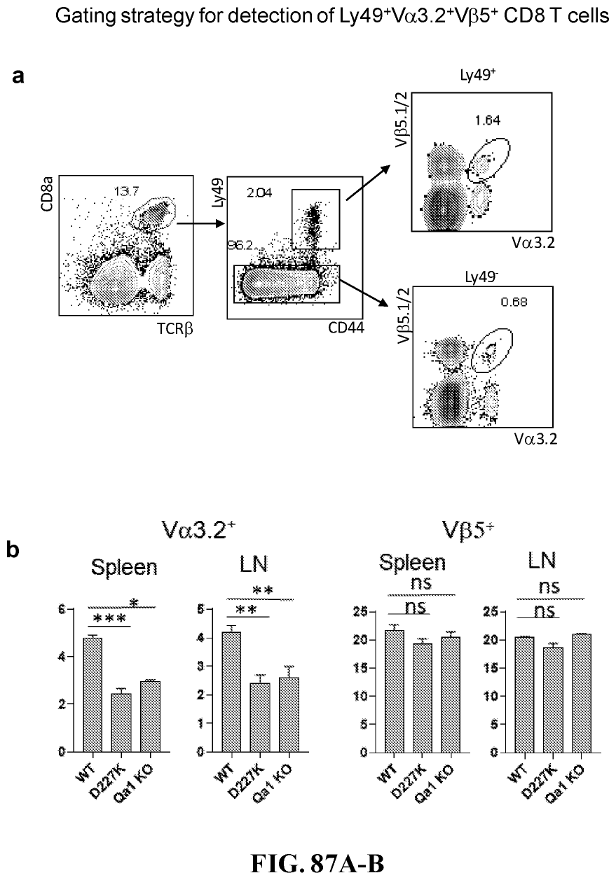

We then asked whether Qa-1-restricted CD8 Treg in non-transgenic mice might also express the Vα3.2 and Vβ5 TCR pair noted above. We found that triad* (Ly49⁺ CD122⁺ CD44⁺) Vα3.2⁺/Vβ5.1,2⁺ CD8 Treg were reduced by 60-80% in mice carrying a Qa-1 deletion or Qa-1 D227K point mutation (FIG. 79D, FIG. 87A-B). In contrast, the numbers or percent of Vα3.2⁻/Vβ5.1,2⁻ triad+CD8 T cells were not affected by either a Qa-1 deletion or mutation (FIG. 79D). Moreover, residual triad+Vα3.2⁺/Vβ5.1,2+CD8 Treg in Qa-1 KO mice displayed a 50-75% reduction in the Ki67 proliferation marker (FIG. 79D). Taken together, these findings indicate that Qa-1-restricted CD8 Treg that express the Ly49⁺ CD44⁺ CD122⁺ marker triad are also distinguished from MHC class Ia restricted CD8 Treg by expression of a conserved set of TCRs that are equipped to recognize pQa-1 complexes that include structurally-unrelated self-peptides.

### CD8 Treg Phenotype of Qa-1-FL9 Specific T Cells

To gain further insight into the contribution of TCR usage to the differentiation and function of self-reactive CD8 Treg, we cloned each of the 12 TCR pairs specific for Qa-1-FL9 into retroviral vectors and expressed them in 58C (α⁻β⁻) hybridoma cells. Expression of each TCR in 58C cells was accompanied by specific binding to Qa-1-FL9 but not Qa-1-Hsp60 tetramers (FIG. 48A, 48B), presumably reflecting the peptide-specific CDR3 sequences noted above (FIG. 46A-B). The binding avidity of each Qa-1-FL9-specific TCR was then determined according to a dose-response analysis of the concentration of FL9 peptides required for CD69 upregulation by each transduced hybridoma (FIG. 48C). A Qa-1-FL9-specific TCR with an intermediate (FL9.2) avidity and high avidity (FL9.8) for Qa-1-FL9 (FIG. 79E) were defined further in an antigen dissociation assay, which confirmed the higher affinity of the FL9.8 TCR, as judged by increased retention of Qa-1-FL9 tetramer compared with the FL9.2 TCR (FIG. 79F).

We generated Tg mice that express FL9.2 and FL9.8 self peptide-specific TCRs using methods employed previously to generate OT-I TCR Tg mice that depended on insertion of (pES.42.1c and pKS913.CD18.31) vectors⁹. We then generated BM chimeras after reconstitution of lethally-irradiated B6 hosts with BM transduced with OT-I, FL9.2 or FL9.8 TCRs to study the contribution of these TCRs to the selection and development of CD8 Treg. The percent of Tg TCR⁺ T cells in peripheral tissues was ˜90% in the three BM chimeras that had been reconstituted with each TCR transgene (FIG. 79G, FIG. 0A). Analysis of thymocytes revealed that approximately 20% of FL9.2 and 40% of FL9.8 thymocytes expressed markers of negative selection that included caspase 3 and PD1, while OT-I thymocytes did not express these negative selection markers (FIG. 79G, FIG. 50A). Analysis of peripheral T cells revealed that the two FL9 TCR transgenes but not OT-I displayed increased expression of CD44 and Ki67 (FIG. 79H,FIG. 50B) and reduced levels of TCR and CD8, i.e., a CD8 T cell phenotype associated with chronic activation by self antigens^{10,11} (FIG. 50C). Chronic exposure of CD8 T cells to self-antigen may also upregulate expression of NKG2D receptors^{12,13}, which has been correlated with immunoregulatory function¹⁴. FL9.8 T cells displayed an age-dependent upregulation of NKG2D expression (>80% at 4 mo), while FL9.2 T cells displayed a more modest increase (20-40%) (FIG. 49A-B).

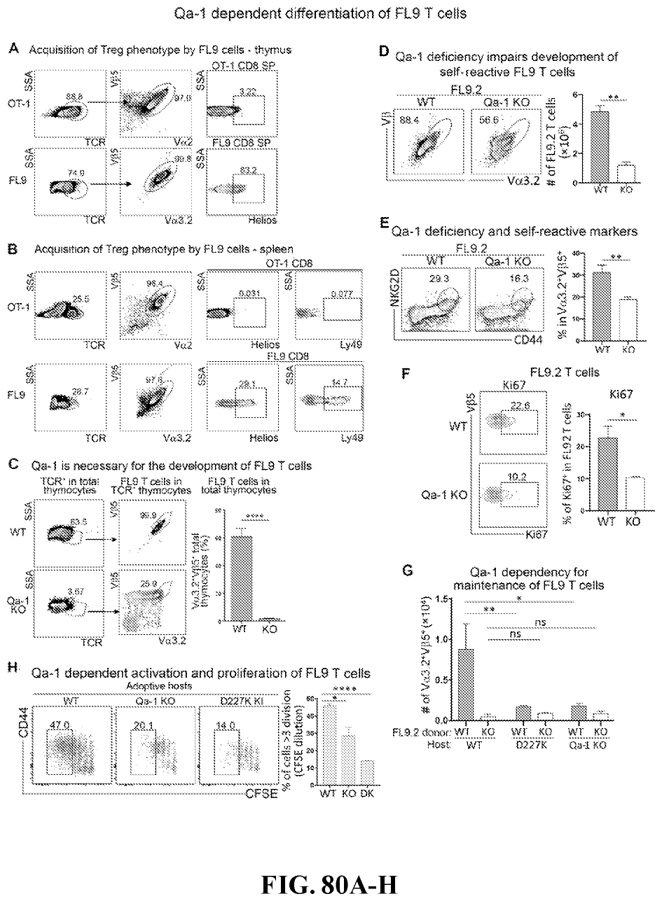

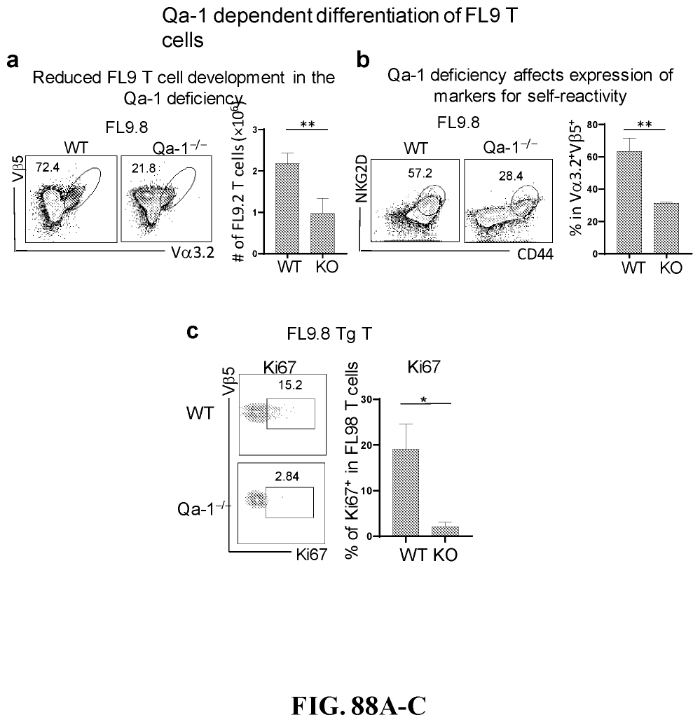

We then asked whether FL9 T cells acquire and maintain a characteristic Treg phenotype described here and previously 1. All TCR⁺ cells in the thymus express the Tg TCRα and TCRP (Vα3.2, Vβ5) similar to levels of developing OT-I thymocytes (Vα2, Vβ5), while only FL9 T cells expressed the canonical CD8 Treg TF Helios (FIG. 80A). After maturation, peripheral FL9 T cells maintained this characteristic Treg phenotype and expressed both Helios TF and Ly49 (FIG. 80B). We then defined the contribution of Qa-1 according to the development of CD8 Treg in Qa-1 WT and Qa-1 KO FL9.2 Tg mice. In the absence of the Qa-1 restriction element, FL9 T cells did not develop: only ˜1% of thymocytes in the Qa-1 KO thymus expressed the FL9 TCR, compared to ˜60% of thymocytes that expressed FL9 TCR in WT Tg mice (FIG. 80C). This data indicates that Qa-1 is the single restriction MHC molecule for FL9 T cells rather than the crossreactivity of this TCR with other classical and non-classical MHC molecules indicated previously^{7,15,16} Deletion of the Qa-1 restriction element also resulted in an approximate 80% reduction in the numbers of FL9.2 CD8 T cells (FIG. 80D), as well as higher affinity FL9.8 TCR⁺ T cells in the spleen (FIG. 88A). Moreover, expression of the CD44 and NKG2D receptors (FIG. 80E, FIG. 88B)) and Ki67 proliferation marker was markedly reduced in both FL9.2 (FIG. 80F) and FL9.8 T cells (FIG. 88C) in peripheral lymphoid tissues.

Although the numbers of TCR Tg FL9.2 T cells were reduced by 70-80% in mice that expressed defective or deleted Qa-1, a significant fraction remained. We asked whether these residual TCR Tg CD8 cells in the spleen and lymph node of Qa-1-deficient mice were functionally impaired. Transfer of residual FL9.2 T cells from Qa-1 KO mice into irradiated adoptive Qa-1 WT hosts revealed that very few (˜10%) survived compared with the robust survival of FL9 T cells from Qa-1 WT donors (FIG. 80G). We then asked whether recognition of Qa-1 in peripheral tissues was essential for continued survival of mature Qa-1-restricted FL9 T cells that had initially differentiated in a Qa-1-sufficient (Qa-1 WT) environment. We found that transfer of FL9 CD8 T cells from Qa-1 WT donors into Qa-1 KO or D227K KI hosts (that express a Qa-1 D227K point mutation that impairs the interaction between Qa-1 and the CD8 co-receptor) displayed poor survival, similar to that of CD8 TCR Tg cells transferred from Qa-1-deficient donors (FIGS. 80G-H). These data indicate that Qa-1 expression is essential for both initial intrathytmic development of Qa-1 restricted CD8 Treg and survival.

### Detection and Elimination of Antigen-Specific CD4 Cells by Qa-1-Restricted CD8 Treg

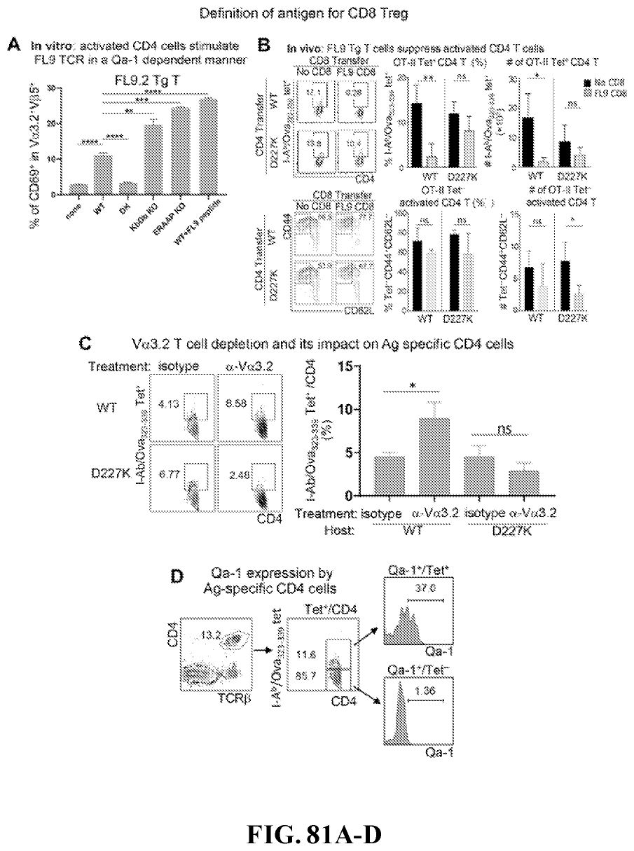

Although targeting of CD4 cells by CD8 Treg may reflect TCR-dependent recognition of pQa-1 complexes expressed by activated CD4 cells¹, the nature of the target complexes is not well understood. Here we asked whether FL9-Qa-1 complexes represent a major functional target on Ag-specific CD4 T cells. Analysis in vitro indicated that FL9 TCR Tg T cells are efficiently stimulated by activated CD4 T cells from B6 (Qa-1 WT) mice but not by B6.Qa-1-D227K KI mice (FIG. 81A, FIGS. 60A, 61A). Moreover, Kb‘Db’ CD4 cells provoked increased responses by FL9 TCR⁺ CD8 T cells, reflecting the absence of a dominant Qdm default peptide derived from MHC class Ia that competitively binds to Qa-1 (FIG. 81A, FIGS. 54A, 54A). Activated ERAAP-deficient CD4 cells strongly stimulated FL9 TCR Tg T cells, consistent with increased Qa-1-FL9 expression by cells lacking the ERAAP enzyme, which normally destroys this peptide 7 (FIG. 81A, FIGS. 54A, 55A and 55B). Collectively, these findings indicate that activated CD4 cells express an ERAAP-sensitive Qa-1-FL9 ligand that is recognized by Qa-1-FL9-specific CD8 Treg.

CD4 cells that express high affinity TCRs may co-express high levels of Qa-1¹,17 To examine whether CD8 Treg may selectively target activated CD4 T cells with high affinity for immunizing or environmental antigen, we characterized CD4 cells generated after immunization according to expression of Qa-1-FL9 and sensitivity to inhibition by CD8 Treg. We transferred CD4 cells from OT-II-peptide-immunized WT B6 or B6-D227K mice into B6 hosts with or without FL9 TCR Tg CD8 cells, followed by immunization with OT-II/CFA. Analysis of OT-II tetramer* CD4 cells, which represent CD4 cells with the highest avidity for immunizing OVA, revealed that co-transfer of FL9 TCR Tg CD8 T cells inhibited more than 90% of OVA tetramer* CD4 cells (FIG. 81B, upper panel), while virtually all activated (CD44⁺ CD62L−) CD4 cells that were tetramer-negative were spared (FIG. 81B, lower panel). Suppression of Ag-specific CD4 cells depended on Qa-1 targeting, since FL9 CD8 T cells suppressed the response of B6 (WT) CD4 T cells but not B6-D227K CD4 T cells (FIG. 81B, upper panel). These data indicate that a) the Qa-1-FL9 peptide complex is expressed on a substantial proportion of CD4 cells after activation by Ag, and b) CD4 cells expressing highly avid TCR (tetramer*) for cognate Ag strongly express Qa-1 (Qa-lhi) and are efficiently suppressed by Qa-1-restricted CD8 Treg.

Based on observations that Qa-1-restricted CD8 Treg mainly express the Vα3.2/Vb5 pair (FIG. 56), we asked whether Ab-mediated depletion of Vα3.2⁺ T cells after immunization to OVA might enhance the Ag-specific CD4 T cell responses. Virtually complete depletion of Vα3.2⁺ T cells could be achieved after anti-Vα3.2 Ab administration (FIG. 56). B6.WT but not B6.Qa-1 D227K mice immunized with OVA/CFA and boosted with OVA/IFA developed an increased frequency of OVA-specific CD4 cells (I-Ab/Ova323-339 Tet⁺) after depletion of Vα3.2⁺ T cells (FIG. 81C). The markedly increased Qa-1 levels expressed by I-A^{b}/Ova₃₂₃-3₃₉ Tet⁺ CD4 cells confirms previous findings that CD8 Treg can selectively target Qa-1^{hi}0 Ag-specific CD4 cells (FIG. 81D). These data also indicate that Qa-1-restricted CD8 cell targeting by anti-TCRa3.2 Ab can be employed to modulate CD8 Treg activity.

### Definition of FL9-Superagonistpeptides

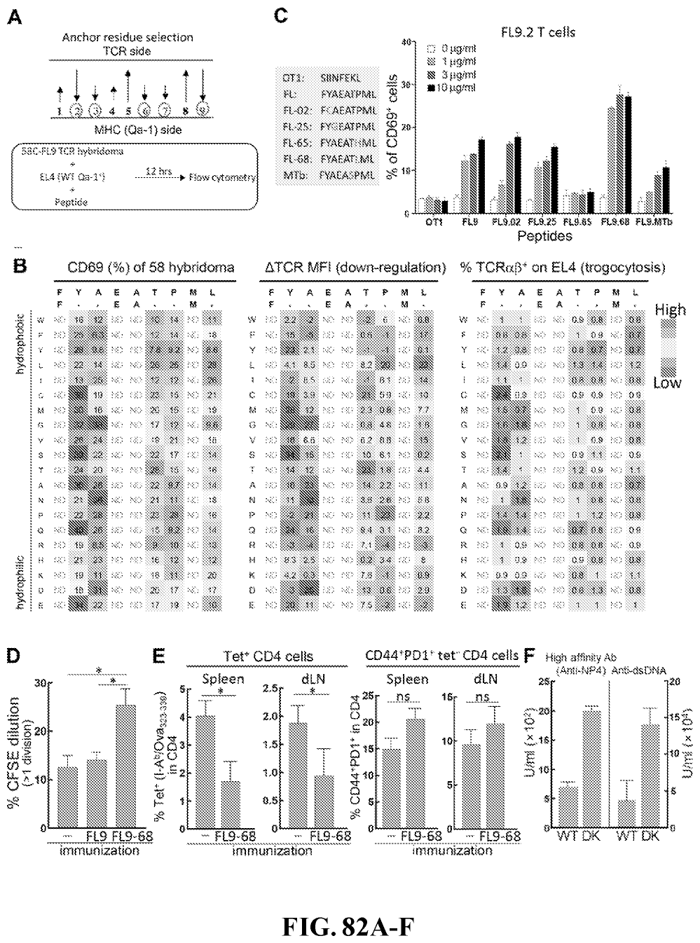

Since chronically-activated CD4 cells express Qa-1-FL9 complexes, in vivo expansion of Qa-1-FL9 specific CD8 Treg may promote elimination of these CD4 T cells in clinical settings. However, immunization of mice with the FL9 self-peptide did not elicit detectable expansion of CD8 Treg (FIG. 57), perhaps reflecting the relatively low Qa-1 binding affinity and consequent weak TCR activation by this self peptide¹,1⁸,1⁹ We reasoned that efficient mobilization of Qa-1 restricted CD8 Treg-specific for self-peptides might require immunization with peptide analogs with increased Qa-1 binding activity. To systematically improve binding stability to Qa-1, we screened a peptide library composed of ˜100 aa-exchange variants of FL9 peptide at MHC-anchoring positions 2, 3, 6, 7 and 9 (FIG. 82A). The FL9 TCR⁺ 58C hybridoma was incubated with EL4 (Qa-1⁺) cells that had been pulsed with FL9 peptide variants and monitored for CD69 upregulation and TCR downregulation to define the stimulatory activity of each peptide variant (FIG. 82B, left and middle panels). The interaction of Qa-1-peptide complexes with the FL9 TCR was also evaluated by measurement of TCR trogocytosis, which reports the strength of TCR binding to defined pMHC ligands²⁰ (FIG. 82B, right panel). Variant FL9 peptides that stimulated increased FL9 TCR⁺ hybridoma responses compared with the native FL9 peptide (as judged by CD69 expression, TCR downregulation and increased trogocytosis) were then subjected to dose-response analysis.

This analysis revealed that a FL9 peptide variant containing a P→L substitution at pos 7—termed FL9-68—displayed markedly enhanced dose-dependent stimulatory activity for FL9.2 and FL9.8 TCRs compared with the cognate FL9 self-peptide (FIG. 82C, FIG. 58C). Immunization with the FL9-68 agonist peptide activated FL9 TCR⁺ CD8 T cells after transfer into congeneic (CD45.1⁺ B6) hosts or TCRa¹ hosts compared with the native FL9 peptide (FIG. 82D). Immunization of CD45.1⁺ B6 hosts with Ova₃₂₃₋₃₃₉ in CFA followed by analysis of activated CD4 T cells revealed that FL9-68 vaccination inhibited the response of I-A^{b}/Ova₃₂₃₋₃₃₉ tet⁺ CD4 cells by >50% but did not reduce the numbers or percent of non-specifically activated CD4 cells that did not bind to the FL9-Qa-1 tetramer (FIG. 82E). The finding that CD8 Treg mainly target Tet⁺ CD4 cells (Qa-lhi) was consistent with the observation that defective CD8 Treg activity in Qa-1.D227K mutant mice resulted in a significant increase in high affinity Ab responses to immunizing Ags (NP-Ova) as well as anti-dsDNA Abs (FIG. 82F). Collectively, these findings indicate that FL9-specific CD8 Treg preferentially suppress Qa-1^{hi} CD4 cells that express high avidity TCR for the immunizing antigen without generalized immune suppression and indicate that the FL9-68 peptide analog could be used to deliberately mobilize CD8 Treg in clinical settings.

### Peptide-Dependent Mobilization of CD8 Treg and Inhibition of Allo-Immunity

The observation that CD8 Treg mainly target high affinity CD4 cells indicated that mobilization of CD8 Treg may allow suppression of destructive autoimmune- or allo-responses without generalized immune suppression and the concomitant risk of increased vulnerability to pathogenic infection. Antibody-mediated rejection (AMR) remains a major barrier to successful solid organ transplantation. Since pathogenic alloantibodies mediating AMR are produced mainly by GC B cells after induction by Tfh cells²¹, increased expression of the Qa-1-FL9 complex by activated Tfh cells may allow targeting and suppression of pathogenic CD4 cells by Ag-specific CD8 Treg. Indeed, our recent analysis of the allograft response of B6.Qa-1 mutant (B6.Qa-1-D227K) mice indicated that disruption of the interaction between CD8 Treg and Qa-1 in recipients of a fully allogenic heart transplant model results in unchecked Tfh cell proliferation and accelerated Ab-mediated allograft injury ²².

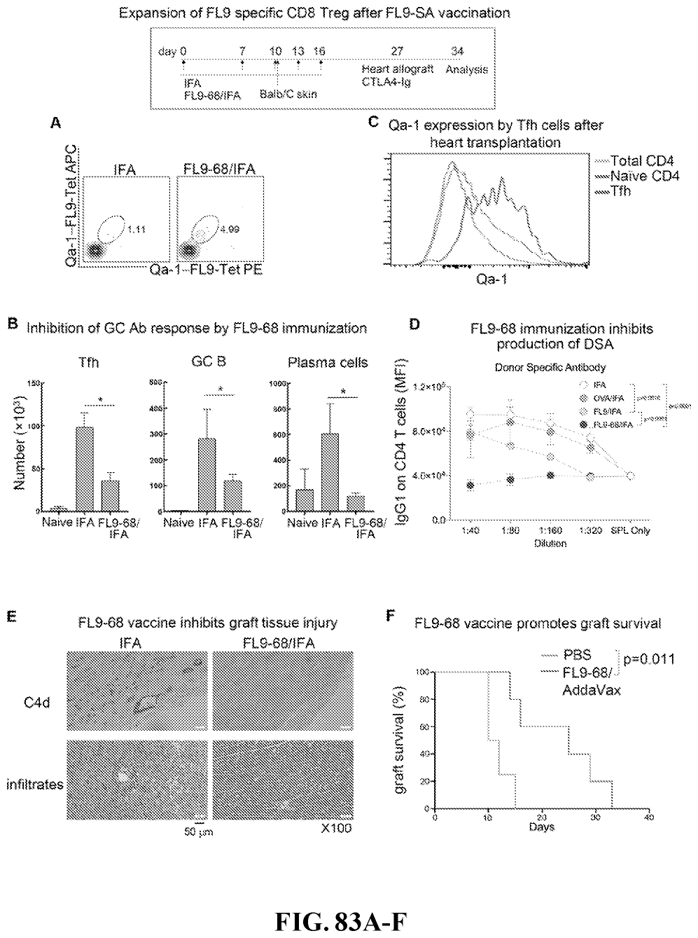

We transplanted heart allografts into previously skin allograft-sensitized hosts, since heart transplants into non-sensitized hosts induce strong cellular rejection but only weak humoral responses. To test the ability of FL9-specific CD8 Treg to inhibit humoral allograft rejection, we first asked whether expansion of FL9-specific T cells by the FL9-68 peptide might suppress the anti-heart graft response²³. FL9-68 peptide vaccination of B6 mice bearing a Balb/C skin allograft displayed a 4-5-fold increase of Ly49F* FL9-specific CD8 cells (FIG. 83A). Allo-antigen sensitized B6 hosts (from Balb/C kin allografts) were transplanted with Balb/C heart allografts, which normally are rejected secondary to a strong anti-graft antibody response. Seven days later, we noted reduced GC responses in B6 hosts that had been vaccinated with FL9-68/IFA but not IFA alone, along with reduced numbers of Tfh cells (PD-1⁺ CXCR5⁺ CD4⁺), activated GC B cells (FAS+GL-7⁺B220⁺), and plasma cells (B220-CD138⁺) (FIG. 83B). Suppression of the GC response after FL9-68 administration was also associated with increased numbers of Qa-lhi Tfh cells (FIG. 83C), reduced donor-specific antibody (DSA) responses and markedly diminished graft pathology, as measured by C4d deposition and level of immune cell infiltration (FIGS. 83D-E). The impact of CD8 Treg mobilization by FL9-68 peptide was also evident from the significantly prolonged graft survival times after FL9-68 peptide vaccination compared to adjuvant administration alone (FIG. 83F).

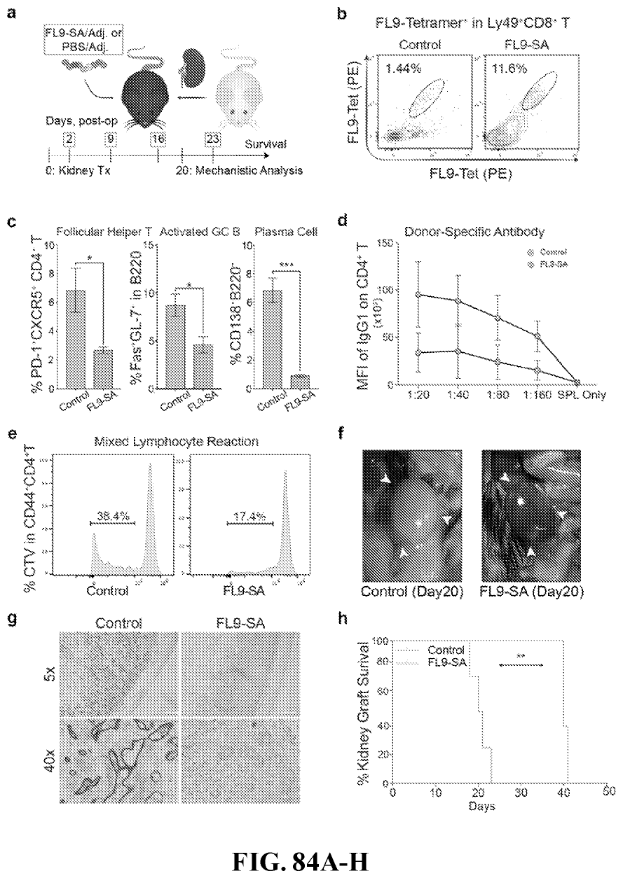

The effect of peptide-superagonist-mediated expansion of CD8 Treg on anti-allograft immunity against fully-mismatched kidney transplants was also tested (FIG. 84A). Analysis of allograft-draining lymph nodes on day 20 following kidney transplantation (n=5-7/group) revealed a >7-fold increase in FL9-specific Treg as measured by FL9-Qa-1 tetramers (FIG. 84B). Administration of FL9-68-IFA but not IFA alone markedly reduced the GC response, as judged by reduced frequency of Tfh cells (PD-1⁺CXCR5⁺ CD4⁺), activated GC B cells (FAS*GL-7⁺B220⁺), and plasma cells (B220-CD138*) (FIG. 84C), as well as a significant decrease in DSA (FIG. 84D). Incubation of Celltrace Violet™-stained CD4⁺ T cells from (B6) hosts with (irradiated) donor (Balb/c) lymphocytes also revealed that FL9-68 vaccination reduced CD44⁺ CD4 T-cell memory responses (FIG. 84E). Vaccination with FL9-68 induced significant protection from antibody-mediated allograft rejection, as judged by reduced C4d deposition in the peritubular capillaries of kidney allografts (FIG. 84F-G) and substantial prolongation of allograft survival compared to controls (mean survival time: 20.5 vs. 40 days) (FIG. 84H).

Collectively, these data indicate that peptide-based mobilization of CD8 Treg that recognize pathogenic CD4 Tfh cells represents a promising therapeutic approach for drug-free reduction of Ab-mediated injury in a murine model of heart and kidney allografts.

### Discussion

There is increasing evidence that suppression of pathogenic host responses by CD8 Treg depends on precise recognition of MHC class Ib-self-peptide complexes expressed by activated CD4 effector cells i,z,24 However, the basis for this recognition and targeting of chronically-activated CD4 T cells has been obscure. Our characterization of TCRs expressed by Qa-1-restricted CD8 Treg revealed a surprisingly restricted expression of CDR1/CDR2 regions expressed by both TCRα and 3 chains. This interaction may allow Qa-1-restricted CD8 T cells specific for diverse self-peptides to escape peptide-mediated negative selection in the thymus and equip them to survey CD4 T cells that express high affinity TCRs for immunizing antigens and strongly upregulate Qa-1. This preferential TCR usage by CD8 Treg specific for self-peptides expressing a TCR transgene was apparent in polyclonal Qa-1-restricted CD8 Treg. Virtually all Qa-1-restricted CD8 Treg in the polyclonal CD8 Treg population express Vα3.2/V5 and were dramatically reduced in mice that carried a Qa-1 deletion or mutation that impaired their interaction with pQa-1. In contrast, Ly49F* Helios±CD8 T cells that did not express Vα3.2/Vβ5 were not affected by altered Qa-1 expression.

Analysis of development of immature thymocytes that expressed a Vα3.2/Vβ5 TCR transgene specific for Qa-1-FL9 self-peptide indicated acquisition of the CD8 Treg phenotype in the thymus and periphery that depended on expression of Qa-1. Indeed, the residual populations of FL9-TCR⁺ CD8 T cells that persisted in the absence of Qa-1 displayed markedly reduced survival and impaired activation in adoptive environments that expressed a WT Qa-1 phenotype (FIG. 80G-H).

MHC-E-restricted unconventional CD8 T cells have been shown to develop into both effector and regulatory lineages¹,2⁵,2⁶ Our findings indicate that expression of distinct sets of TCR may be a decisive event in guiding immature CD8 thymocytes into regulatory lineage-specific development rather than effector CD8 T cell development. We indicate that MHC-E-restricted CD8 T cells that express TCRs that recognize self-peptides may differentiate into mature CD8 T cells that express canonical features of Treg, including Helios and Ly49 as well as a central memory phenotype, reflecting their continuous recognition of self-antigen. Definition of the canonical TCR pairs expressed by CD8 Treg allows for selective activation or deletion of these MHC-E-restricted CD8 Treg by antibodies specific for these TCR and modulation of their activity in pathologic conditions that include autoimmune disease and cancer.

The TCR-based recognition noted above may account for precise elimination of CD4 T cells that express high avidity TCRs for cognate Ag. Elevated expression of the Qa-1-FL9 complex by activated CD4 cells may allow sensitive monitoring for increased Ag-activated but not non-specifically activated CD4 T cells by CD8 Treg. More than 90% of relatively high avidity tetramer* CD4 T cells were eliminated, while non-specifically activated tetramer-negative CD4 cells were spared (FIG. 81A-D). Robust upregulation of Qa-1 by tet+CD4 T cells may allow efficient targeting of the major cellular source of helper function, for B-cell-dependent Ab responses, i.e., Tfh cells, without generalized immune suppression 1.

CD8 Treg express relatively low levels of CD8 and TCR, reflecting their self-reactivity (FIG. 50C), and may be significantly more anergic than T cells specific for foreign antigens. These considerations indicate that specific mobilization of CD8 Treg requires more self-peptide variants with increased agonistic activity²⁸,2⁹. We screened synthetic FL9 peptides containing altered residues at Qa-1 anchoring positions to identify agonists with enhanced binding to pMHCI and increased immunogenicity. The FL9-68 peptide variant, which includes a P→L amino acid exchange at position 7, displayed significantly enhanced stimulatory activity in vitro and in vivo. Activation of CD8 Treg by FL9-68 peptide resulted in CD8 Treg expansion that resulted in inhibition of Tfh cell and GC B cell responses and reduced production of anti-graft Abs in heart and kidney transplantation models and prolonged organ survival (FIG. 83A-F, FIG. 84A-G).

Multiple autoimmune diseases have been associated with autoantibody generation secondary to dysregulated high affinity Tfh expansion⁴,3⁰,31 CD8 Treg-mediated control of autoAb generation is an essential mechanism for inhibition of autoimmune disease development 1. In contrast to CD4 Treg, we show here that CD8 Treg can be expanded and activated in a pMHC-specific fashion to efficiently target CD4 Th cells with high affinity for cognate antigen, including self-Ags. While we have used a peptide-dependent strategy to mobilize CD8 Treg, characteristic expression of TCR Va/VP by CD8 Treg might also be exploited for activation and expansion of CD8 Treg in vivo. Activation of CD8 Treg via anti-TRAV Abs (targeting conserved CDR1/CDR2) may mobilize a broad repertoire of CD8 Treg to efficiently inhibit or eliminate pQa-1hi pathogenic CD4 cells. The efficacy of CD8 Treg expansion followed by inhibiton of autoAb generation and accompanying pathology can be tested in mouse models of autoimmune disease, including EAE, TlD (NOD) and SLE (BXSB-Yaa). Expression of the nonclassical MHC gene products (MHC-E) in murine (Qa-1) models and in humans (HLA-E) are both limited to two alleles, unlike the highly polymorphic classical MHC genes 1. Identification of homologous TCR expressed by human CD8 Treg 3 may allow selective mobilization of HLA-E-restricted human CD8 Treg for treatment of antibody-mediated pathologic conditions.

### Methods

Mice C57BL/6 (B6), B6.SJL-PtprcaPepcb/BoyJ (B6.CD45.1), Balb/C, C57BL/6-Tg(TcraTcrb)1100Mjb/J (OT-1), B6.129P2-H2-K1^{tmlBpe}H2-D1^{tmlBpe}/DcrJ (Kb^{-/-}Db^{-/-}), and B6.129S2-TCRa^{tmlMom}/J (TCRα^{-/-}) mice were obtained from the Jackson laboratory (Bar Harbor ME). B6.Qa-1.D227K KI and B6.Qa-1^{-/-} (B6.129S6-H2-T23^{tMlcant}/J) mice were generated in the laboratory and previously described ^{24,32,33} FL9.2, FL9.8 TCR Tg mice were generated in the laboratory as described below and maintained on a Qa-1 WT and KO background. ERAAP^{-/-} mice were provided by Dr. Kenneth Rock (UMASS Medical Center, Worcester). All experiments were performed in compliance with institutional guidelines as approved by the Animal Care and Use Committee of the Dana-Farber Cancer Institute (DFCI) and the Brigham & Women's Hospital.

### Antibodies and Flow Cytometry

Fluorescence-labelled antibodies for TCRb (clone: H57-597), CD3e (17A2), CD44 (IM7), CD122 (TM-b1), Ly49C/I/F/H (14B11), Vα3.2 (RR3-16), Vα2 (B20.1), Vb5.1/5.2 (MR9-4), CD4 (RM4-5), CD8a (53-6.7), CD8b (YTS156.7.7), CD69 (H1.2F3), Qa-1b (6A8.6F10.1A6), PD-1 (29F.1A12), Act. Caspase 3 (5A1E), Ki67 (16A.8), B220 (RA3-6B2), Fas (SA367H8), CXCR5 (SPRCL5), FoxP3 (FJK-16S), NKG2D (A10) and NKG2A (20d5) were purchased from BD Biosciences, eBioscience and Biolegend. For the detection of FL9 T cells, Qa-1b/FL9-PE, Qa-1b/FL9-APC, Qa-1b/Hsp60p2l6-PE, Qa-1b/Hsp60p2l6-APC tetramers were generated by NIH tetranmer core facility and provided for this study. I-A^{b}/Ova₃₂₃₋₃₃₉ tetramers were purchased from MBL International.

### Identification and Isolation of FL9 Specific TCR

Bone marrow derived DCs were generated from Kb^{-/-} Db^{-/-} mice in the presence of 20 ng/ml GM-CSF. 6 days later, DCs were stimulated with 50 ng/ml LPS for 12 hrs. DCs were irradiated (30 Gy) and pulsed with FL9 peptide by incubating with 10 mg/ml FL9 peptides for 2 hrs at 37° C. FL9-loaded Kb^{-/-} Db^{-/-} DCs were injected into WT B6 mice at day 0, 8 and 15. At day 22, Qa-1b/FL9 Tet⁺ cells were detected in the CD44⁺ CD122⁺Ly49⁺ CD8 subset and single Tet⁺ cells sorted by FACS. Identification of TCRα and TCRb chains for each sorted cells was performed according to a previously published protocol ³⁴. In brief, one-step RT-PCR was performed by adding RT-PCR mix to each well. Primers for the RT-PCR mix include the leader sequences and constant region sequences of TCRs where adapter sequences were added to the 5′ end of the leader primers³⁴(FIG. 62). cDNA from this RT-PCR was used to amplify TCRα and TCRb separately using the nested PCR principle. The PCR products were sequenced using mTRAC_1st2R and mTRBC_1 st2R primers for TCRa and TCRb amplicons respectively and analyzed with the IMGT/V-Quest algorithm (http://www.imgt.org).

### Generation of TCR⁺ Hybridoma and TCR Affinity Test

The cDNAs encoding the TCRα and TCRβ chain were inserted into the pMIG vector that contains GFP cassette, which was transfected into the PLAT-E cells using FuGENE6 (Promega). The culture medium was replaced with the fresh medium in 24 hrs and supernatant was collected 72 hrs after transfection and used to transduce TCR^{-/-} 58C hybridoma. Expression of TCRα and TCRβ pairs on the surface of 58C hybridoma was analyzed by staining with Qa-1b-FL9 tetramers, anti-CD3ε and anti-TCR Vβ Abs. Relative affinity of FL9.8 and FL9.2 TCR was analyzed by measuring the tetramer staining decay kinetics 35. FL9.8 TCR⁺ and FL9.2 TCR⁺ hybriboma were incubated with PE conjugated Qa-1b/FL9 tetramers in the presence of anti-Qa-1 Abs. Cells were fixed at different time points (0-120 min) after initiation of incubation and the intensity of PE staining was measured as an indication of tetramer binding by flow cytometry.

### Generation of FL9 TCR Tg Mice

FL9.2 and FL9.8 TCR transgenes were generated by replacing the TCR V(D)J elements of the pES.42.1c and pKS913.CD18.31 vectors that have been used previously to generate OT-I TCR Tg mice ⁹ with each TCRα and TCRβ cDNAs fragments for FL9.2 and FL9.8 TCRs. The vector was linearized and used to target C57BL/6 ES cells using standard methods at the Transgenic Core Facility at Beth Israel Deaconess Medical Center. Founder lines for the FL9.2 and FL9.8 TCR Tg mice were established after genotyping with the following primers: common primer set for both FL9.2 and FL9.8 TCRα 5′-CTAGAAGACTCAGGGTCTGA-3′ and 5′-TCGGCACATTGATTTGGGAGTCA-3′ amplified 1kbp for the transgene, a primer set for FL9.2 TCRb 5′-ACACTGTCCTCGCTGATTCTG-3′ and 5′-GATGTGAATCTTACCGAGAACAGTCAGTCTGGTTC-3′ and a primer set for FL9.8 TCRβ5′-TAACACTGTCCTCGCTGAC-3′ and ATACAGCGTTTCTGCACTAG-3′ both amplified 500 bp for transgene.

### Peptide Mutageneisis and Superagonist Peptide Screen

A peptide library was generated by single mutation of each Qa-1 anchoring position (p 2, 3, 6, 7 and 9) of the FL9 peptide (FYAEATPML) with 20 aa, which is composed of 96 FL9 variant peptides. FL9.8 TCR⁺ 58C hybridomas were incubated with EL4 cells that were pulsed with each FL9 variant. After 12 hrs, CD69 expression and levels of TCR expression were measured by flow cytometry. For analysis of the binding strength of FL9 TCR with Qa-1-FL9 variants, trogocytosis was measured directly by the detection of FL9 TCR (Vα3.2⁺Vβ5⁺) on EL4 cells. FL9.8 TCR⁺ 58C hybridomas were co-cultured with EL4 cells that were pulsed with FL9 variant peptides from the library. After 2 hrs, the percentage of Vα3.2⁺Vα5⁺EL4 cells was assessed by flow cytometry as a measurement of trogocytosis.

### Adoptive Transfer and In Vivo Suppression Assay

Qa-1.WT or Qa-1.D227K KI mice were immunized i.p. with 100 μg Ova₃₂₃₋₃₃₉ peptide in CFA. 7 days later, 1×10⁵ CD25⁻CD4 cells were isolated from these mice and transferred into WT B6 mice along with FL9 Tg T cells. WT B6 adoptive hosts were immunized on the footpad with 20 μg Ova₃₂₃₋₃₃₉ peptide in CFA. After 7 days, the frequency and numbers of I-A^{b}/Ova₃₂₃₋₃₃₉ tetramer+CD4 cells and activated CD4 cells were assessed in the inguinal and popliteal LNs of B6 hosts by flow cytometry.

### FL9 Peptide Immunization

CD45.1⁺B6 or TCRα^{-/-} mice were transferred with CFSE labelled 2×10⁶ FL9.2 Tg T cells followed by i.p. immunization with 100 μg FL9 or FL9-68 peptides in CFA. Proliferation and activation of FL9.2 Tg T cells in the adoptive hosts were analzyed by assessing CFSE dilution, Ki67 and CD69 expression at day 3 and 6 after transfer.

### Heart Transplantation of Skin-Sensitized Hosts and Analysis of Immune Response

B6 mice were immunized i.p with 50 μg FL9-68-Adjuvant (IFA or AddaVax™) or Adjuvant alone at day 0 and 7 followed by a BALB/C→B6 skin transplant at day 10. FL9-68-Adj or Adj immunization was repeated on days 10, 13 and 16. At day 27, fully vascularized Balb/C hearts were transplanted into the abdominal cavity of B6 mice using microsurgical techniques, as previously described³⁶. Heart graft survival was determined by monitoring palpable heart beating. At day 16 after skin sensitization, levels of FL9 T cells, Tfh, GC B and plasma cells in dLNs were analyzed by flowcytometry. Serum was collected from the heart graft recipient B6 mice that were either immunized with FL9-68/IFA or IFA alone at day 16. Serially diluted serum was incubated with 1×10⁶ donor splenocytes in total volume 100 l PBS for 30 min followed by detection of surface bound Abs on CD4 cells using anti-CD4 (Biolegend, Clone RM4-5) and anti-mouse IgG1 Abs (BD Biosciences, Clone A85-1). Histological analysis of heart grafts was performed by InvivoEx company using anti-C4d Ab (Hycult Biotech) and Vector Blue Alkaline Phosphatase Substrate Kit (Vector Laboratories). For the mixed lymphocyte rejection, CD25⁻CD4 T cells were isolated from draining lymph nodes of B6 hosts and co-cultured with irradiated Balb/c donor splenocytes. Proliferation was measured by immunofluoresnce with Celltrace violet^{™}.

### Kidney Transplantation and Analysis of Immune Response

The left kidney of BALB/c mice (H-2^{d}) was recovered using a full-length ureter and transplanted into a B6 host (H-2^{b}). The ureter of the remaining native kidney was then ligated on post-operative day 2-4 to inhibit native kidney function. Surgical success was determined if mice survived seven days post-surgery (POD). Transplanted B6 hosts were treated intraperitoneally with FL9-SA (50 μg), or PBS emulsified in Adjuvant (Addavax™), once a week starting POD2. On day 20 following kidney transplantation (n=5-7/group), allograft draining lymphoid tissues were assessed for FL9-specific Treg (Qa-1-FL9 Tet⁺), Tfh, GC B and plasma cells, DSA levels in sera, capillary C4d deposition and gross anatomy of kidney allografts. Survival of kidney allografts was measured by survival of recipients with absence of native kidney function.

### Statistical Analysis

Prism v.9.0 (GraphPad Software) was used for statistical analyses. Statistical significance was calculated according to Wilcoxon-Mann-Whitney rank sum test for comparison of two conditions; Kruskal-Wallis test was performed for comparison of more than two conditions. A P value of <0.05 was considered to be statistically significant (* =<0.05, ** =<0.01, *** =<0.001, **** =<0.0001).

### REFERENCES

1 Nakagawa, H., Wang, L., Cantor, H. & Kim, H. J. New Insights Into the Biology of CD8 Regulatory T Cells. Adv Immunol 140, 1-20, doi:10.1016/bs.ai.2018.09.001 (2018).
2 Saligrama, N. et al. Opposing T cell responses in experimental autoimmune encephalomyelitis. Nature 572, 481-487, doi:10.1038/s41586-019-1467-x (2019).
3 Li, J. et al. KIR(+)CD8(+) T cells suppress pathogenic T cells and are active in autoimmune diseases and COVID-19. Science 376, eabi9591, doi:10.1126/science.abi9591 (2022).
4 Kim, H. J. et al. CD8⁺ T regulatory cells express the Ly49 class I MHC receptor and are defective in autoimmune-prone B6-Yaa mice. Proc.Natl.Acad.Sci.U.S.A 108, 2010-2015 (2011).
5 Vivier, E. & Anfossi, N. Inhibitory NK-cell receptors on T cells: witness of the past, actors of the future. Nat.Rev.Immunol 4, 190-198 (2004).
6 Lazaro, E. et al. Differential HIV epitope processing in monocytes and CD4 T cells affects cytotoxic T lymphocyte recognition. J Infect Dis 200, 236-243, doi:10.1086/599837 (2009).
7 Nagarajan, N. A., Gonzalez, F. & Shastri, N. Nonclassical MHC class Ib-restricted cytotoxic T cells monitor antigen processing in the endoplasmic reticulum. Nat.Immunol. 13, 579-586 (2012).
8 Leavenworth, J. W., Tang, X., Kim, H. J., Wang, X. & Cantor, H. Amelioration of arthritis through mobilization of peptide-specific CD8⁺ regulatory T cells Journal of Clinical Investigation 123, 1382-1389 (2013).
9 Hogquist, K. A. et al. T cell receptor antagonist peptides induce positive selection. Cell 76, 17-27 (1994).
10 Schonrich, G. et al. Down-regulation of T cell receptors on self-reactive T cells as a novel mechanism for extrathymic tolerance induction. Cell 65, 293-304, doi:10.1016/0092-8674(91)90163-s (1991).
11 Xiao, Z., Mescher, M. F. & Jameson, S. C. Detuning CD8 T cells: down-regulation of CD8 expression, tetramer binding, and response during CTL activation. J Exp Med 204, 2667-2677, doi:10.1084/jem.20062376 (2007).
12 Zloza, A. et al. Engagement of NK receptor NKG2D, but not 2B4, results in self-reactive CD8⁺ T cells and autoimmune vitiligo. Autoimmunity 44, 599-606, doi:10.3109/08916934.2011.593599 (2011).
13 Dhanji, S., Teh, S. J., Oble, D., Priatel, J. J. & Teh, H. S. Self-reactive memory-phenotype CD8 T cells exhibit both MHC-restricted and non-MHC-restricted cytotoxicity: a role for the T-cell receptor and natural killer cell receptors. Blood 104, 2116-2123, doi:10.1182/blood-2004-01-0150 (2004).
14 Dai, Z. et al. Normally occurring NKG2D+CD4⁺ T cells are immunosuppressive and inversely correlated with disease activity in juvenile-onset lupus. J Exp Med 206, 793-805, doi:10.1084/jem.20081648 (2009).
15 Guan, J., Peske, J. D., Taylor, J. A. & Shastri, N. The nonclassical immune surveillance for ERAAP function. Curr Opin Immunol 70, 105-111, doi:10.1016/j.coi.2021.05.008 (2021).
16 Guan, J., Yang, S. J., Gonzalez, F., Yin, Y. & Shastri, N. Antigen Processing in the Endoplasmic Reticulum Is Monitored by Semi-Invariant alphabeta TCRs Specific for a Conserved Peptide-Qa-1(b) MHC Class Ib Ligand. J Immunol 198, 2017-2027, doi:10.4049/jimmunol.1600764 (2017).
17 Fazilleau, N., McHeyzer-Williams, L. J., Rosen, H. & McHeyzer-Williams, M. G. The function of follicular helper T cells is regulated by the strength of T cell antigen receptor binding. Nat Immunol 10, 375-384, doi:10.1038/ni.1704 (2009).
18 Kambayashi, T. et al. The nonclassical MHC class I molecule Qa-1 forms unstable peptide complexes. J.Immunol 172, 1661-1669 (2004).
19 van Hall, T., Oliveira, C. C., Joosten, S. A. & Ottenhoff, T. H. The other Janus face of Qa-1 and HLA-E: diverse peptide repertoires in times of stress Microbes Infect 12, 910⁻⁹¹⁸ (2010).
20 Li, G. et al. T cell antigen discovery via trogocytosis. Nat Methods 16, 183-190, doi:10.1038/s41592-018-0305-7 (2019).
21 Kwun, J. et al. Crosstalk Between T and B Cells in the Germinal Center After Transplantation. Transplantation 101, 704-712, doi:10.1097/TP.0000000000001588 (2017).
22 Choi, J. Y. et al. Regulatory CD8 T cells that recognize Qa-1 expressed by CD4 T-helper cells inhibit rejection of heart allografts. Proc Natl Acad Sci USA 117, 6042-6046, doi:10.1073/pnas.1918950117 (2020).
23 Baldwin, W. M., 3rd, Valujskikh, A. & Fairchild, R. L. Antibody-mediated rejection: emergence of animal models to answer clinical questions. Am J Transplant 10, 1135-1142, doi:10.1111/j.1600-6143.2010.03065.x (2010).
24 Kim, H. J., Verbinnen, B., Tang, X., Lu, L. & Cantor, H. Inhibition of follicular T helper cells by CD8⁺ Treg is essential for self tolerance Nature 467, 328-332 (2010).
25 Hansen, S. G. et al. Broadly targeted CD8⁺ T cell responses restricted by major histocompatibility complex E. Science, doi:10.1126/science.aac9475 (2016).
26 Bian, Y. et al. MHC Ib molecule Qa-1 presents Mycobacterium tuberculosis peptide antigens to CD8⁺ T cells and contributes to protection against infection. PLoS pathogens 13, e1006384, doi:10.1371/journal.ppat.1006384 (2017).
27 Tubo, N. J. et al. Single naive CD4⁺ T cells from a diverse repertoire produce different effector cell types during infection. Cell 153, 785-796, doi:10.1016/j.cell.2013.04.007 (2013).
28 Yu, Z. et al. Poor immunogenicity of a self/tumor antigen derives from peptide-MHC-I instability and is independent of tolerance. J Clin Invest 114, 551-559, doi:10.1172/JC121695 (2004).
29 Yu, W. et al. Clonal Deletion Prunes but Does Not Eliminate Self-Specific alphabeta CD8(+) T Lymphocytes. Immunity 42, 929-941, doi:10.1016/j.immuni.2015.05.001 (2015).
30 Mishra, S. et al. TGF-beta and Eomes control the homeostasis of CD8+regulatory T cells. J Exp Med 218, doi:10.1084/jem.20200030 (2021).
31 Serr, I. & Daniel, C. Regulation of T Follicular Helper Cells in Islet Autoimmunity. Front Immunol 9, 1729, doi:10.3389/fimmu.2018.01729 (2018).
32 Hu, D. et al. Analysis of regulatory CD8 T cells in Qa-1-deficient mice. Nat Immunol 5, 516-523 (2004).
33 Lu, L. et al. Regulation of activated CD4⁺ T cells by NK cells via the Qa-1-NKG2A pathway Immunity 26, 593-604 (2007).
34 Hamana, H., Shitaoka, K., Kishi, H., Ozawa, T. & Muraguchi, A. A novel, rapid and efficient method of cloning functional antigen-specific T-cell receptors from single human and mouse T-cells. Biochem Biophys Res Commun 474, 709-714, doi:10.1016/j.bbrc.2016.05.015 (2016).
35 Savage, P. A., Boniface, J. J. & Davis, M. M. A kinetic basis for T cell receptor repertoire selection during an immune response. Immunity 10, 485-492, doi:10.1016/s1074-7613(00)80048-5 (1999).
36 Cai, S. et al. Prolonged Mouse Cardiac Graft Cold Storage via Attenuating Ischemia-Reperfusion Injury Using aNew Antioxidant-Based Preservation Solution. Transplantation 100, 1032-1040, doi:10.1097/TP.0000000000001079 (2016).

### Example 11

### Depletion of CD8 Treg Enhances Anti-Tumor Immunity

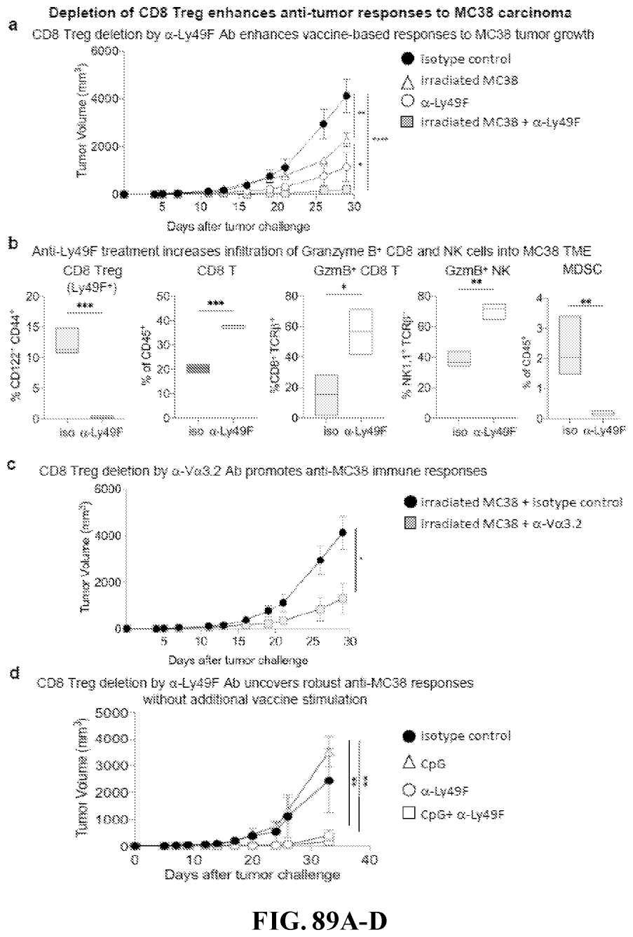

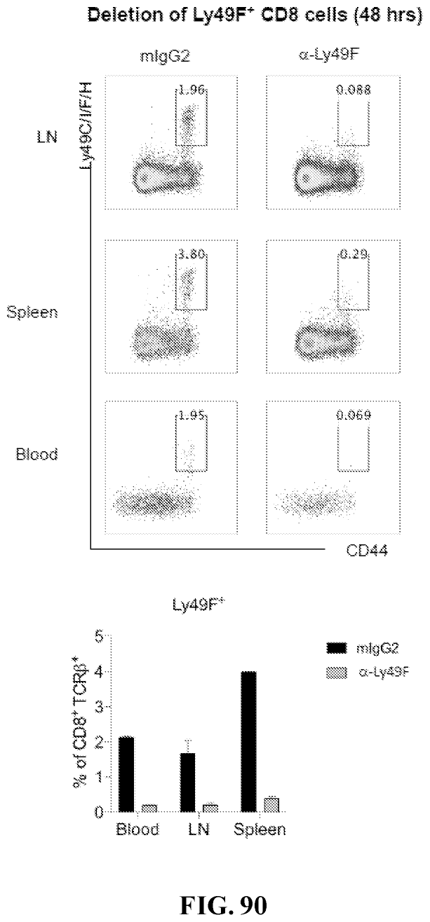

While not wishing to be held to a mechanism, the suppressive function of Qa-1-restricted CD8 Treg on activated T cells indicates that anti-tumor immune responses might be enhanced by a reduction in CD8 Treg levels. Expression of the Ly49F surface marker by CD8 Treg (but not other lymphocytes, including NK cells⁴) allowed us to deplete Ly49F+CD8 Treg by >95% in spleen, lymphocytes and blood (FIG. 90). We initially asked whether depletion of CD8 Treg might enhance anti-tumor immune responses along with vaccination with irradiated tumor cells in response to the syngeneic MC38 murine colon carcinoma. Vaccination alone led to a modest delay of MC38 growth. Depletion of CD8 Treg by a-Ly49F administered after tumor growth was detectable beginning at day 8 alone or combined with vaccine prevented significant tumor growth (FIG. 89A), indicating that CD8 Treg depletion represents a promising therapeutic option for derepressing anti-tumor immune responses. This view was supported by analysis of the MC38 TME, which revealed that a-Ly49F Ab treatment led to increased numbers of both total and GzmB+CD8 and NK cells, along with a decrease in immunosuppressive MDSC (FIG. 89B). Moreover, depletion of the dominant clones of Qa-1-restricted CD8 Treg that express TCR Vα3.2 (see FIG. 56 for depletion efficiency of a-Vα3.2 Ab) provided a similar increase in anti-tumor immunity (FIG. 89C).

We then tested the impact of CD8 Treg depletion under conditions that elicit a Thl-biased immune response by injection (s.c.) of CpG-ODN²⁴ 3 days after inoculation of MC38 cells. Although treatment with CpG-ODN did not reveal a significant therapeutic effect, CD8 Treg depletion at day 8-10 either alone or with CpG-ODN treatment strongly inhibited tumor growth (FIG. 89D).

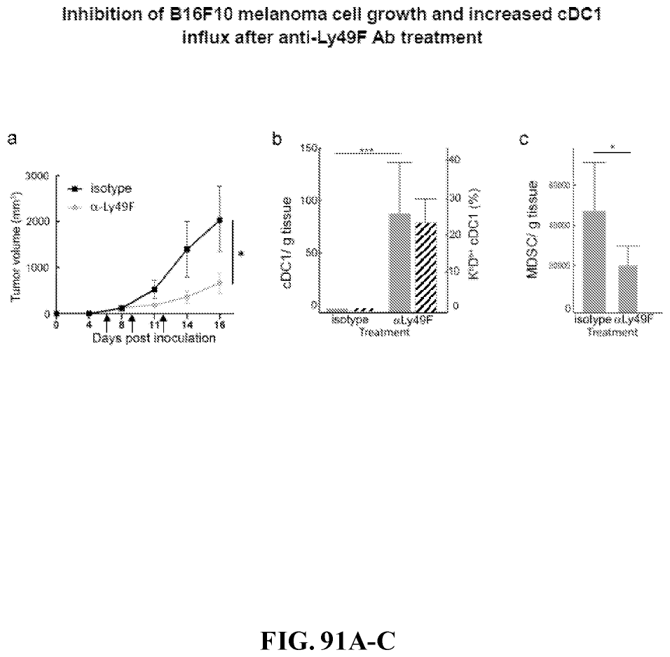

Finally, we asked whether CD8 Treg depletion might enhance the response to a second syngeneic tumor, the B16F10 melanoma. In this case, we were also able to examine the early changes in the TME (day 15) to determine whether skewing of the DC response towards cDC1 enrichment during tumor growth was present early in the response. We noted that B16F10 tumor growth was accompanied by a marked increase in cDC1 at the expense of MDSC (FIGS. 91A-C). Together, these data indicate that strategies that target CD8 Treg-dependent immune suppression may represent an effective approach, as monotherapy or in combination with other immunotherapies, to enhance the immune response against cancers.

Depletion of CD8 Treg may augment anti-tumor immune responses (FIGS. 89A-D). Administration of a-Ly49F or a-Vα3.2 Ab enhanced anti-tumor responses leading to inhibition of growth by the syngeneic MC38 murine colon carcinoma. Analysis of the B16F10 melanoma model also indicated an early increase in type 1 cDC, which may be essential for initiation of durable T cell responses³³. Although the major cellular target of CD8 Treg within the TME is not well-defined, increased expression of Qa-1 by activated CD4 cells, CD8 T cells and cDC in the TME represent targets. While not wishing to be held to a mechanism, depletion of human CD8 Treg using anti-KIR Ab may improve current immunotherapy protocols. Identification of TCR homologs to the murine Vα3.2/Vβ5.1 set expressed by human CD8 Treg ³ to allow selective mobilization or depletion of HLA-E-restricted human CD8 Treg may form the basis of new and effective treatments for disorders that reflect deficient or excessive immune responses.

### Tumor Inoculation and Ab Treatment

C57BL/6 (B6) mice (8-12 wk old) were inoculated s.c. with 2×10⁵ MC38 cells. Mice were treated with tumor cell vaccine (10⁶ MC38 cells irradiated 2000 rads [20Gy]), α-Ly49F Ab alone or combined with tumor cell vaccine (n=5-6/group). Irradiated MC38 tumor cells were injected subcutaneously on day 7-10 on the opposite flank of the MC38 tumor inoculation site. Anti-Ly49F or isotype control (30 mg/mouse) was administered on days 8, 10 and 13 post MC38 tumor cell inoculation. For vaccination, MC38-Cas9 cells were transduced with lentivirus containing Ezh2 gRNA (5′-AGAGTACATTATGGCACCG-3′) at MOI 0.5 in the presence of 2.5 mg/ml puromycin for 72 hrs. A portion of the resulting polyclonal EZH2^{KO} containing 10⁻²⁰% EZH2^{KO} cells were subcloned and highly enriched EZH2^{KO} cells were irradiated (2000 R) and used for vaccination. In some experiments, MC38 cells containing a 10⁻²⁰% EZH2^{KO} cells, which displayed growth curves that were not significantly different from MC38 WT cells, were used as test tumor inocula to increase expression of MHC and immunogenicity³⁹. For CpG-ODN treatment, WT B6 mice were injected with CpG-ODN (50 mg/mouse) on day 3 followed by treatment with α-Ly49F Abs (30 mg/mouse) on days 8, 11, 14 and 17.

### REFERENCES

4. Kim, H. J. et al. CD8⁺ T regulatory cells express the Ly49 class I MHC receptor and are defective in autoimmune-prone B6-Yaa mice. Proc.Natl.Acad.Sci. U.S.A 108, 2010-2015 (2011).
24. Hirobe, S. et al. Adjuvant Activity of CpG-Oligonucleotide Administered Transcutaneously in Combination with Vaccination Using a Self-Dissolving Microneedle Patch in Mice. Vaccines (Basel) 9, doi:10.3390/vaccines9121480 (2021).
33. Dahling, S. et al. Type 1 conventional dendritic cells maintain and guide the differentiation of precursors of exhausted T cells in distinct cellular niches. Immunity 55, 656-670 e658, doi:10.1016/j.immuni.2022.03.006 (2022).
39. Burr, M. L. et al. An Evolutionarily Conserved Function of Polycomb Silences the MHC Class I Antigen Presentation Pathway and Enables Immune Evasion in Cancer. Cancer Cell, doi:10.1016/j.ccell.2019.08.008 (2019).

### EQUIVALENTS

Those skilled in the art will recognize, or be able to ascertain, using no more than routine experimentation, numerous equivalents to the specific substances and procedures described herein. Such equivalents are considered to be within the scope of this invention.

What is claimed:

1. A method for mobilizing a CD8 Treg cell in a mammal, comprising administering a CD8 Treg stimulator to the mammal.

2. The method of claim 1, wherein the administering decreases CD4 T cell activity in the mammal.

3. The method of claim 1, wherein the mammal comprises a mouse.

4. The method of claim 3, wherein the CD8 Treg cell expresses CD8, Ly49 and CD44.

5. The method of claim 3, wherein the CD8 Treg cell expresses CD8, Ly49, CD44 and CD122.

6. The method of claim 3, wherein the CD8 Treg cell is MHC class Ib restricted.

7. The method of claim 3, wherein the CD8 Treg cell can suppress CD4 cells in a Qa-1 dependent manner.

8. The method of claim 1, wherein the mammal comprises a human.

9. The method of claim 8, wherein the CD8 Treg cell expresses CD8, an inhibitory KIR (iKIR) and CD44.

10. The method of claim 8, wherein the CD8 Treg cell expresses CD8, an iKIR, CD44 and CD122.

11. The method of claim 8, wherein the CD8 Treg cell is MHC class Ib restricted.

12. The method of claim 8, wherein the CD8 Treg can suppress CD4 cells in an HLA-E dependent manner.

13. The method of claim 1, wherein the CD8 Treg stimulator comprises a peptide/polypeptide agonist or superagonist of the CD8 Treg cell.

14. The method of claim 13, wherein the peptide agonist binds to a T cell receptor (TCR) on the CD8 Treg cell and to an MHC class Ib molecule on a CD4 T cell.

15. The method of claim 14, wherein the MHC class Ib molecule comprises Qa-1 in mice or HLA-E in humans.

16. The method of claim 13, comprising an amino acid sequence FSNEATLML, WYADVTPAL; or an amino acid sequence 88%, 89%, 90%, 91%, 92%, 93%, 94%, 95%, 96%, 97%, 98% or 99% identical thereto.

17. The method of claim 13, comprising an amino acid sequence: FYAEATLML (FL9-68); or an amino acid sequence 88%, 89%, 90%, 91%, 92%, 93%, 94%, 95%, 96%, 97%, 98% or 99% identical thereto.

18. The method of claim 13, comprising an amino acid sequence: IMLDTEIRL (BO-1), FMNDALLFL (BO-2), FMEEYMPFL (BO-3), FMEDAGPRL (BO-5), WMSEDHTLL (BO-6), VMQDEKSRL (BO-9), ISSEDGVPL (BO-10), or an amino acid sequence 88%, 89%, 90%, 91%, 92%, 93%, 94%, 95%, 96%, 97%, 98% or 99% identical thereto.

19. The method of claim 13, comprising an amino acid sequence: FISDSFFFL (Endo 9), FYAEGTTML (MTb) or an amino acid sequence 88%, 89%, 90%, 91%, 92%, 93%, 94%, 95%, 96%, 97%, 98% or 99% identical thereto.

20. The method of claim 13, comprising an amino acid sequence: FYAEATPML (FL9) or an amino acid sequence 88%, 89%, 90%, 91%, 92%, 93%, 94%, 95%, 96%, 97%, 98% or 99% identical thereto.

21. The method of claim 13, wherein the administration of the polypeptide can suppress CD4 cells in the mammal.

22. The method of claim 21, wherein the polypeptide is conjugated to a lipophilic albumin binding tail conjugate.

23. The method of claim 1, wherein the CD8 Treg stimulator comprises an antibody that binds to a CD8 Treg cell.

24. The method of claim 23, wherein the antibody binds to a TCR on the CD8 Treg cell.

25. The method of claim 24, wherein the TCR on the CD8 Treg cell binds a self-peptide.

26. The method of claim 24, wherein the TCR on the CD8 Treg cell binds peptides that can bind to an MHC Ib molecule.

27. The method of claim 26, wherein the MHC Ib molecule comprises Qa-1 or HLA-E.

28. The method of any one of claims 25 or 26, wherein the self-peptide or peptide comprises an amino acid sequence selected from the group consisting of WYADVTPAL, FYAEATLML (FL9-68), IMLDTEIRL (BO-1), FMNDALLFL (BO-2), FMEEYMPFL (BO-3), FMEDAGPRL (BO-5), WMSEDHTLL (BO-6), VMQDEKSRL (BO-9), ISSEDGVPL (BO-10), FISDSFFFL (Endo 9), FYAEGTTML (MTb), FYAEATPML (FL9) and an amino acid sequence 88%, 89%, 90%, 91%, 92%, 93%, 94%, 95%, 96%, 97%, 98% or 99% identical thereto.

29. The method of any one of claims 25 or 26, wherein the self-peptide or peptide is from Hsp60.

30. The method of claim 29, wherein the peptide from Hsp60 comprises GMKFDRGYI (Hsp60p2l6) or an amino acid sequence 88%, 89%, 90%, 91%, 92%, 93%, 94%, 95%, 96%, 97%, 98% or 99% identical thereto.

31. The method of claim 24, wherein the antibody binds to any one of the CDRs illustrated in FIGS. 27-28 and 29-30.

32. The method of claim 23, wherein the antibody comprises a bispecific antibody that binds to Ly49 or iKIR and to CD8, or binds to Ly49 or iKIR and to a TCR of the CD8 Treg cells.

33. The method of claim 32, wherein the antibody comprises a bispecific antibody that binds to iKIR and to CD8, or to iKIR and a TCR of the CD8 Treg cells.

34. The method of claim 1 wherein the mammal receives a transplanted organ.

35. The method of claim 34, wherein the method diminishes humoral and/or cellular rejection of the transplanted organ.

36. The method of claim 35, wherein humoral rejection of the organ comprises antibody mediated rejection (AMR).

37. The method of claim 1, wherein the mammal has an autoimmune disorder.

38. A method for treating organ rejection or for treating an autoimmune disease in a patient, comprising decreasing CD4 T cell activity in the patient.

39. A method for treating organ rejection or for treating an autoimmune disease in a patient, comprising mobilizing CD8 Treg cells in the patient.

40. The method of any one of claims 38 or 38 comprising administering a CD8 Treg stimulator to the patient.

41. A method for depleting CD8 Treg cells in a mammal, comprising administering to the mammal a CD8 Treg cell depleter.

42. The method of claim 41, wherein the CD8 Treg cell depleter comprises an antibody that binds to the CD8 Treg cells.

43. The method of claim 41, wherein the administering stimulates CD4 T cell activity in the mammal.

44. The method of claim 42, wherein the antibody depletes the CD8 Treg cells.

45. The method of claim 42, wherein the antibody binds to CD8 Treg cells that express CD8, Ly49 (mouse) and CD44, and/or binds to CD8 Treg cells that express CD8, an iKIR (human) and CD44.

46. The method of claim 42, wherein the antibody binds to Ly49 and/or iKIR on the CD8 Treg cells.

47. The method of claim 42, wherein the antibody binds to a TCR on the CD8 Treg cells.

48. The method of claim 47, wherein the TCR on the CD8 Treg cells binds a self-peptide.

49. The method of claim 48, wherein the self-peptide comprises an amino acid sequence selected from the group consisting of WYADVTPAL, FYAEATLML (FL9-68), IMLDTEIRL (BO-1), FMNDALLFL (BO-2), FMEEYMPFL (BO-3), FMEDAGPRL (BO-5), WMSEDHTLL (BO-6), VMQDEKSRL (BO-9), ISSEDGVPL (BO-10),: FISDSFFFL (Endo 9), FYAEGTTML (MTb), FYAEATPML (FL9) and an amino acid sequence 88%, 89%, 90%, 91%, 92%, 93%, 94%, 95%, 96%, 97%, 98% or 99% identical thereto.

50. The method of claim 48, wherein the self-peptide or peptide is from Hsp60.

51. The method of claim 50, wherein the peptide from Hsp60 comprises GMKFDRGYI (Hsp60p216) or an amino acid sequence 88%, 89%, 90%, 91%, 92%, 93%, 94%, 95%, 96%, 97%, 98% or 99% identical thereto.

52. The method of claim 47, wherein the antibody binds to any one of the CDRs illustrated in FIGS. 27-28 and 29-30.

53. The method of claim 41, wherein the CD8 Treg cell depleter comprises a bispecific antibody that binds to Ly49 or iKIR and to CD8, binds to Ly49 or iKIR and to a TCR, or binds to CD8 and to a TCR of the CD8 Treg cells.

54. The method of claim 42, wherein the antibody comprises a bispecific antibody that binds to iKIR and to CD8, or to iKIR and a TCR of the CD8 Treg cells.

55. The method of claim 42 wherein the antibody has an Fc portion that can bind to an Fc receptor (FcR).

56. The method of claim 55, wherein the Fc portion binds to the FcR on the surface of an effector cell.

57. The method of claim 55, wherein the FcR comprises an Fc-gamma receptor (FcγR), an Fc-alpha receptor (FcαR), or an Fc-epsilon receptor (FcεR).

58. The method of claim 57, wherein the FcγR comprises FcγRI, FcγRII, or FcγRIII.

59. The method of claim 56, wherein the effector cell kills the CD8 Treg cells.

60. The method of claim 56, wherein the effector cell comprises a natural killer (NK) cell or a macrophage.

61. The method of claim 56, wherein antibody has a modified Fc portion that has increased binding to the effector cell as compared to an antibody that has an Fc portion that is not modified.

62. The method of claim 41, wherein the mammal has cancer.

63. The method of claim 41, wherein the method increases anti-tumor activity in the mammal

64. A method for treating cancer in a patient, comprising increasing CD4 T cell activity in the patient.

65. A method for treating cancer in a patient, comprising depleting CD8 Treg cells in the patient.

66. The method of any one of claims 64 or 65, comprising administering to the patient, an antibody that depletes CD8 Treg cells.

67. The method of claim 66, wherein the antibody is used in combination with a therapeutic cancer vaccine.

68. The method of claim 66, wherein the antibody is used in combination with an immune checkpoint inhibitor or checkpoint inhibitor.

69. The method of claim 68, wherein the immune checkpoint inhibitor comprises a PD-L1 inhibitor.

70. An isolated antibody or antigen binding fragment thereof that binds to an inhibitory killer cell Ig-Like receptor (iKIR).

71. The isolated antibody or antigen binding fragment thereof of claim 70, wherein the iKIR comprises KIR3DL1, KIR2DL2, or KIR3DL3.

72. An isolated antibody or antigen binding fragment thereof that binds to a TCR of a CD8 Treg cell.

73. The isolated antibody or antigen binding fragment of claim 72, wherein the TCR binds to a peptide having an amino acid sequence selected from the group consisting of WYADVTPAL, FYAEATLML (FL9-68), IMLDTEIRL (BO-1), FMNDALLFL (BO-2), FMEEYMPFL (BO-3), FMEDAGPRL (BO-5), WMSEDHTLL (BO-6), VMQDEKSRL (BO-9), ISSEDGVPL (BO-10), FISDSFFFL (Endo 9), FYAEGTTML (MTb), FYAEATPML (FL9) and an amino acid sequence 88%, 89%, 90%, 91%, 92%, 93%, 94%, 95%, 96%, 97%, 98% or 99% identical thereto, in the context of an MHC Ib molecule.

74. The method of claim 72, wherein the TCR binds to a peptide from Hsp60, in the context of an MHC Ib molecule.

75. The method of claim 74, wherein the peptide from Hsp60 comprises GMKFDRGYI (Hsp60p216) or an amino acid sequence 88%, 89%, 90%, 91%, 92%, 93%, 94%, 95%, 96%, 97%, 98% or 99% identical thereto.

76. The isolated antibody or antigen binding fragment of claim 73 or 74, wherein the MHC Ib molecule comprises Qa-1 or HLA-E.

77. The isolated antibody or antigen binding fragment of claim 73 or 74, wherein the MHC Ib molecule comprises HLA-E.

78. The isolated antibody or antigen binding fragment of claim 76, wherein the MHC Ib molecule is on a CD4 cell.

79. The isolated antibody or antigen binding fragment thereof of claim 72, that binds to:
a CD8 Treg TCRα CDR1 sequence at least 90% identical to YFGTPYY;
a CD8 Treg TCRα CDR2 sequence at least 90% identical to YYPGDPVV;
a CD8 Treg TCRα CDR3 sequence at least 90% identical to AVSIWATSSGQKLV; AVTRYGSSGNKLI, AVRANYAQGLT, AVRGQGRALI, AVKDSGYNKLT, AVSSNNAGAKLT, AVRANTGKLT, AVKGGNYKPT, or AVKSTGSKLS;
a CD8 Treg TCRβ CDR1 sequence at least 90% identical to NSQYPW, SGHSN, or SGHLS;
a CD8 Treg TCRβ CDR2 sequence at least 90% identical to LRSPGDK, HYEKVER, or HYDKMER; or
a CD8 Treg TCRβ CDR3 sequence at least 90% identical to TCSARQGSGNTLY, ASSRRPASAETLY, ASSPRLGSAETLY, ASSHRSFSGNTLY, ASSLTGAYEQY, ASSLAGREQY, ASSPGPSQNTLY, ASSLLGGPSAETLY, or ASSPRLGSAETLY.

80. The isolated antibody or antigen binding fragment thereof of claim 72, that binds to:
a CD8 Treg TCRα CDR1 sequence at least 90% identical to ATSIAYPN, or YFGTPL;
a CD8 Treg TCRα CDR2 sequence at least 90% identical to KVITAGQ, or KYYPGDPV;
a CD8 Treg TCRα CDR3 sequence at least 90% identical to ALGEASSGSWQL, AVSSNYNVL, AVSRANTGKL, AVSKDSGYNKL, or AVSKSTGSKL;
a CD8 Treg TCRβ CDR1 sequence at least 90% identical to TNNHN, ISGHL, or LSGHS;
a CD8 Treg TCRβ CDR2 sequence at least 90% identical to; SYGAGS, HYDKME, or HYEKVE; or
a CD8 Treg TCRβ CDR3 sequence at least 90% identical to CASGTGDERL, CASSLVSGSAEQ, CASSLAGREQ, CASSLGQGNYAEQ, or CASSRANYEQ.

81. The antibody or fragment of claim 70 or 72, wherein the antibody comprises a single chain antibody.

82. The antibody of claim 70 or 72, wherein the fragment comprises an antigen-binding fragment (Fab), an Fab′ fragment, an F(ab′)2 fragment, a single chain variable fragment (scFv), or a combination thereof.

83. The isolated antibody or antigen binding fragment thereof of claim 70, comprising a bispecific antibody that binds to iKIR and to CD8, or binds to iKIR and to a TCR of CD8 Treg cells.

84. The isolated antibody or antigen binding fragment thereof of claim 83, comprising a bispecific antibody that binds to iKIR and to a TCR of CD8 Treg cells.

85. An isolated antibody or antigen binding fragment thereof, comprising a bispecific antibody that binds to CD8 and to a TCR of CD8 Treg cells.

86. The isolated antibody or antigen binding fragment thereof of one of claims 84 or 85, that binds to:
a CD8 Treg TCRα CDR1 sequence at least 90% identical to YFGTPYY;
a CD8 Treg TCRα CDR2 sequence at least 90% identical to YYPGDPVV;
a CD8 Treg TCRα CDR3 sequence at least 90% identical to AVSIWATSSGQKLV; AVTRYGSSGNKLI, AVRANYAQGLT, AVRGQGRALI, AVKDSGYNKLT, AVSSNNAGAKLT, AVRANTGKLT, AVKGGNYKPT, or AVKSTGSKLS;
a CD8 Treg TCRβ CDR1 sequence at least 90% identical to NSQYPW, SGHSN, or SGHLS;
a CD8 Treg TCRβ CDR2 sequence at least 90% identical to LRSPGDK, HYEKVER, or HYDKMER; or
a CD8 Treg TCRβ CDR3 sequence at least 90% identical to TCSARQGSGNTLY, ASSRRPASAETLY, ASSPRLGSAETLY, ASSHRSFSGNTLY, ASSLTGAYEQY, ASSLAGREQY, ASSPGPSQNTLY, ASSLLGGPSAETLY, or ASSPRLGSAETLY.

87. The isolated antibody or antigen binding fragment thereof of one of claims 84 or 85, that binds to:
a CD8 Treg TCRα CDR1 sequence at least 90% identical to ATSIAYPN, or YFGTPL;
a CD8 Treg TCRα CDR2 sequence at least 90% identical to KVITAGQ, or KYYPGDPV;
a CD8 Treg TCRα CDR3 sequence at least 90% identical to ALGEASSGSWQL, AVSSNYNVL, AVSRANTGKL, AVSKDSGYNKL, or AVSKSTGSKL;
a CD8 Treg TCRβ CDR1 sequence at least 90% identical to TNNHN, ISGHL, or LSGHS;
a CD8 Treg TCRβ CDR2 sequence at least 90% identical to; SYGAGS, HYDKME, or HYEKVE; or
a CD8 Treg TCRβ CDR3 sequence at least 90% identical to CASGTGDERL, CASSLVSGSAEQ, CASSLAGREQ, CASSLGQGNYAEQ, or CASSRANYEQ.

88. The antibody or antigen binding fragment thereof of any one of claims 70, 72, 83, or 85, wherein the antibody is fully human, humanized, or a chimera.

89. The antibody or antigen binding fragment thereof of any one of claims 70, 72, 83, or 85, further comprising a heavy chain constant region, a light chain constant region, an Fc region, or a combination thereof.

90. The antibody or antigen binding fragment thereof of claim 89, wherein the Fc region is modified to have increased binding to an effector cell as compared to an antibody or fragment that has an Fc portion that is not modified.

91. The antibody or antigen binding fragment thereof of claim 90, wherein the effector cell comprises a natural killer (NK) cell or a macrophage.

92. The antibody or antigen binding fragment thereof of any one of claims 70, 72, 83, or 85, further comprising a therapeutic moiety, an imaging moiety, a capture moiety, or a combination thereof.

93. The antibody or antigen binding fragment thereof of claim 92, wherein:
the therapeutic moiety comprises a toxin;
the imaging moiety comprises a fluorophore, a chromophore, or a combination thereof,
the capture moiety comprises a GST tag, or His-Tag or a combination thereof, or any combination thereof.

94. The antibody or antigen binding fragment thereof of any one of claims 70, 72, 83, or 85, wherein the antibody or fragment is monoclonal.

95. A nucleic acid construct configured to encode the antibody or antigen binding fragment thereof of any one of claims 70, 72, 83, or 85.

96. The isolated antibody or antigen binding fragment thereof of any one of claims 70, 72, 83, or 85, wherein the antibody is configured for use in a CAR-T construct.

97. A recombinant peptide/polypeptide agonist, comprising an amino acid sequence FSNEATLML, WYADVTPAL or an amino acid sequence 88%, 89%, 90%, 91%, 92%, 93%, 94%, 95%, 96%, 97%, 98% or 99% identical thereto.

98. A recombinant peptide/polypeptide agonist, comprising an amino acid sequence: FYAEATLML (FL9-68), or an amino acid sequence 88%, 89%, 90%, 91%, 92%, 93%, 94%, 95%, 96%, 97%, 98% or 99% identical thereto.

99. A recombinant peptide/polypeptide agonist, comprising an amino acid sequence: IMLDTEIRL (BO-1), FMNDALLFL (BO-2), FMEEYMPFL (BO-3), FMEDAGPRL (BO-5), WMSEDHTLL (BO-6), VMQDEKSRL (BO-9), ISSEDGVPL (BO-10), or an amino acid sequence 88%, 89%, 90%, 91%, 92%, 93%, 94%, 95%, 96%, 97%, 98% or 99% identical thereto.

100. A recombinant peptide/polypeptide agonist, comprising an amino acid sequence: FISDSFFFL (Endo 9) or an amino acid sequence 88%, 89%, 90%, 91%, 92%, 93%, 94%, 95%, 96%, 97%, 98% or 99% identical thereto.

101. A recombinant peptide/polypeptide agonist, comprising an amino acid sequence: FYAEATPML (FL9) or an amino acid sequence 88%, 89%, 90%, 91%, 92%, 93%, 94%, 95%, 96%, 97%, 98% or 99% identical thereto.

102. The recombinant polypeptide agonist of any one of claims 97-101, wherein the polypeptide is a CD8 Treg agonist.

103. A pharmaceutical composition comprising a therapeutically effective amount of the antibody of any one of claims 70, 72, 83, or 85 and a pharmaceutically acceptable carrier.

104. A pharmaceutical composition comprising a therapeutically effective amount of the recombinant peptide/polypeptide agonist of any one of claims 97-101 and a pharmaceutically acceptable carrier.

105. A pharmaceutical composition, comprising a combination of the pharmaceutical compositions of one of claims 103 and 104.

106. The pharmaceutical composition of claim 103, wherein the pharmaceutical composition can increase CD4 cell activity in a subject.

107. The pharmaceutical compositions of one of claims 103 or 104, where the pharmaceutical compositions can suppress CD4 cell activity in a subject.

108. The pharmaceutical composition of claim 107, wherein the antibody and/or the recombinant peptide/polypeptide can decrease expression of T follicular helper cells (Tfh), germline center B cells, antibody generation, or a combination thereof.

109. The pharmaceutical composition of claim 107, wherein the antibody and/or the recombinant peptide/polypeptide can decrease production of donor-specific antibodies and/or graft tissue injury.

110. The pharmaceutical composition of claim 107, wherein the antibody and/or the recombinant peptide/polypeptide can mobilize a CD8 Treg cell.

111. The pharmaceutical composition of claim 104, wherein the recombinant peptide/polypeptide is conjugated to a lipophilic albumin binding tail conjugate.

112. A vaccine composition comprising a therapeutically effective amount of a CD8 Treg agonist, wherein the CD8 Treg agonist comprises at least one recombinant polypeptide agonist composition selected from any one of claims 97-101; and a pharmaceutically acceptable carrier, diluent, or excipient.

113. The vaccine composition of claim 112, wherein the CD8 Treg agonist is conjugated to a carrier protein.

114. The vaccine composition of claim 113, wherein the carrier protein comprises a lipophilic albumin binding tail conjugate.

115. The vaccine composition of claim 114, wherein the lipophilic albumin binding tail conjugate comprises 1, 2-Distearoyl-sn-gly cero-3-phosphoethanolamine-Poly(ethylene glycol) (DSPE- PEG).

116. A method of increasing effector CD8 Treg cells in a subject in need thereof, the method comprising administering to a subject:
an effective amount of the antibody of any one of claims 70, 72, 83, or 85;
an effective amount of the polypeptide of any one of claims 13;
the pharmaceutical composition of any one of claims 103-105;
the vaccine composition of claim 112;
or a combination thereof.

117. A method of treating an autoimmune disease or condition, the method comprising administering to a subject in need thereof:
an effective amount of the antibody of any one of claims 70, 72, 83, or 85;
an effective amount of the polypeptide of any one of claims 13;
the pharmaceutical composition of any one of claims 103-105;
the vaccine composition of any one of claims 112;
or a combination thereof.

118. The method of claim 117, wherein the autoimmune disease or condition comprises an antibody-mediated rejection of a transplanted organ, an autoimmune response following an infection, inflammation, or a combination thereof.

119. The method of claim 118, wherein the autoimmune disease comprises systemic lupus erythematosus, multiple sclerosis, type 1 diabetes, or rheumatoid arthritis.

120. A method of treating an antibody-mediated rejection of a transplanted organ in a subject in need thereof, the method comprising administering to a subject in need thereof:
an effective amount of the antibody of any one of claims 70, 72, 83, or 85;
an effective amount of the polypeptide of any one of claims 13;
the pharmaceutical composition of any one of claims 103-105;
the vaccine composition of any one of claims 112;
or a combination thereof.

121. The method of claim 120, wherein the transplanted organ comprises a heart, a kidney, a lung, or a combination thereof.

122. The method of claim 120, further comprising reducing germline center B cell-mediated responses.

123. The method of claim 120, further comprising suppressing autoantibody generation.

124. A method of decreasing effector CD8 Treg cells in a subject in need thereof, the method comprising administering to a subject:
an effective amount of the antibody of any one of claims 70, 72, 83, or 85;
the pharmaceutical composition of claim 103;
or a combination thereof.

125. A method of treating cancer in a subject in need thereof, the method comprising administering to a subject:
an effective amount of the antibody of any one of claims 70, 72, 83, or 85;
the pharmaceutical composition of claim 103;
or a combination thereof.

126. The method of claim 125, wherein the cancer comprises colon cancer, melanoma or lymphoma.

127. The method of any one of claims 116, 117, 120, 124, or 125, wherein the antibody, the polypeptide, the pharmaceutical composition, or the vaccine composition is administered as part of a therapeutic regimen.

128. A method of screening for an autoimmune disorder, the method comprising:
obtaining a sample from a patient,
detecting, in a patient, a biomarker for the autoimmune disorder, the biomarker comprising a T Cell Receptor (TCR) consensus sequence, wherein the T Cell receptor consensus sequence comprises:
a CD8 Treg TCRα CDR1 sequence at least 90% identical to YFGTPYY;
a CD8 Treg TCRα CDR2 sequence at least 90% identical to YYPGDPVV;
a CD8 Treg TCRα CDR3 sequence at least 90% identical to AVSIWATSSGQKLV; AVTRYGSSGNKLI, AVRANYAQGLT, AVRGQGRALI, AVKDSGYNKLT, AVSSNNAGAKLT, AVRANTGKLT, AVKGGNYKPT, or AVKSTGSKLS;
a CD8 Treg TCRβ CDR1 sequence at least 90% identical to NSQYPW, SGHSN, or SGHLS;
a CD8 Treg TCRβ CDR2 sequence at least 90% identical to LRSPGDK, HYEKVER, or HYDKMER; or
a CD8 Treg TCRβ CDR3 sequence at least 90% identical to TCSARQGSGNTLY, ASSRRPASAETLY, ASSPRLGSAETLY, ASSHRSFSGNTLY, ASSLTGAYEQY, ASSLAGREQY, ASSPGPSQNTLY, ASSLLGGPSAETLY, or ASSPRLGSAETLY.

129. A method of screening for an autoimmune disorder, the method comprising:
obtaining a sample from a patient,
detecting, in a patient, a biomarker for the autoimmune disorder, the biomarker comprising a T Cell Receptor (TCR) consensus sequence, wherein the T Cell receptor consensus sequence comprises:
a CD8 Treg TCRα CDR1 sequence at least 90% identical to ATSIAYPN, or YFGTPL;
a CD8 Treg TCRα CDR2 sequence at least 90% identical to KVITAGQ, or KYYPGDPV;
a CD8 Treg TCRα CDR3 sequence at least 90% identical to ALGEASSGSWQL, AVSSNYNVL, AVSRANTGKL, AVSKDSGYNKL, or AVSKSTGSKL;
a CD8 Treg TCR R CDR1 sequence at least 90% identical to TNNHN, ISGHL, or LSGHS;
a CD8 Treg TCR R CDR2 sequence at least 90% identical to; SYGAGS, HYDKME, or HYEKVE; or
a CD8 Treg TCR R CDR3 sequence at least 90% identical to CASGTGDERL, CASSLVSGSAEQ, CASSLAGREQ, CASSLGQGNYAEQ, or CASSRANYEQ.

130. A method of treating an autoimmune disorder comprising:
determining the presence or absence of a biomarker in a patient, wherein the biomarker comprises a T Cell Receptor (TCR) consensus sequence, wherein the T Cell receptor consensus sequence comprises the sequences recited in any one of claims 128 or 129; and
if the biomarker is present, administering to the patient an effective amount of an FL9-like peptide, an anti-inhibitory killer-cell immunoglobulin-like receptor (iKIR) antibody, a TCR antibody against an MHC-1 binding consensus motif, or a combination thereof.

131. The method of claim 130, wherein:
the FL9-like peptide comprises the peptide/polypeptide of any one of claims 97-101;
the anti-inhibitory killer-cell immunoglobulin-like receptor (iKIR) antibody comprises the antibody of any one of claims 70, 83 or 85;
the anti-TCR antibody comprises the antibody any one of claims 72, 83 or 85;
or a combination thereof.

132. A method of screening an antibody of interest for reactivity to CD8 Treg cells, comprising:
contacting the antibody of interest with at least one CD8 Treg cell;
detecting a CD8 Treg cell-antibody complex, wherein the CD8⁺ Treg-antibody complex comprises the antibody of interest bound to a CD8 Treg T cell receptor (TCR) at a complimentary determining region (CDR) 1, CDR2, CDR3, or a combination thereof, as set forth in any one of claims 79 or 80; and
identifying the antibody of interest.

133. The recombinant peptide/polypeptide agonist of any one of claims 97-101, for use as a medicament.

134. The recombinant peptide/polypeptide agonist of any one of claims 97-101, for use as a CD8 Treg cell stimulator in treating an autoimmune disease, autoimmune disorder, or organ rejection in a transplant patient.

135. The isolated antibody or antigen binding fragment of any one of claims 70, 72, 83, or 85, for use as a medicament.

136. The isolated antibody or antigen binding fragment of any one of claims 70, 72, 83, or 85, for use as a CD8 Treg cell stimulator in treating an autoimmune disease, autoimmune disorder, or organ rejection in a transplant patient.

137. The isolated antibody or antigen binding fragment of any one of claims 70, 72, 83, or 85, for use as a CD8 Treg cell depleter in treating cancer in a patient.
